(19) United States
(12) Patent Application Publication
(10) Pub. No.: US 2021/0393742 A1
(43) Pub. Date: Dec. 23, 2021

(54) LONG-ACTING INTERLEUKIN-15 RECEPTOR AGONIST IN COMBINATION WITH ANOTHER PHARMACOLOGICALLY ACTIVE AGENT

(71) Applicant: Nektar Therapeutics, San Francisco, CA (US)

(72) Inventors: Takahiro Miyazaki, San Francisco, CA (US); Loui Madakamutil, San Francisco, CA (US); Saul Kivimae, San Francisco, CA (US)

(21) Appl. No.: 17/292,135

(22) PCT Filed: Nov. 8, 2019
(86) PCT No.: PCT/US2019/060609

§ 371 Date: May 7, 2021

Related U.S. Application Data
(60) Provisional application No. 62/924,015, filed on Oct. 21, 2019.
(60) Provisional application No. 62/848,372, filed on May 15, 2019.
(60) Provisional application No. 62/843,036, filed on May 3, 2019.
(60) Provisional application No. 62/825,437, filed on Mar. 28, 2019.
(60) Provisional application No. 62/818,003, filed on Mar. 13, 2019.
(60) Provisional application No. 62/789,924, filed on Jan. 8, 2019.
(60) Provisional application No. 62/758,344, filed on Nov. 9, 2018.

(51) Int. Cl. A61K 38/20; A61K 47/54; A61K 39/395; A61P 35/00

(52) U.S. Cl. CPC A61K 38/2086; A61K 47/542; A61K 2039/545; A61P 35/00; A61K 39/3955

(57) ABSTRACT

The instant disclosure provides a combination treatment, composition and kit comprising (a) a long-acting IL-15 receptor agonist and (b) one or more antibodies (mAb) targeting a tumor antigen, related methods of preparation and use, for example, in the treatment of conditions responsive to therapy effective to provide, for example, sustained immune activation and/or anti-tumor activity.

### CROSS REFERENCE TO RELATED APPLICATIONS

This application claims the benefit of priority under 35 U.S.C. § 119(e) to U.S. Provisional Patent Application Serial Nos. 62/758,344, filed Nov. 9, 2018; 62/789,924 filed Jan. 8, 2019; 62/818,003 filed Mar. 13, 2019; 62/825,437 filed Mar. 28, 2019; 62/843,036 filed May 3, 2019; 62/848,372 filed May 15, 2019; and 62/924,015 filed Oct. 21, 2019, the disclosures of which are each incorporated herein by reference.

### FIELD

The instant disclosure is directed to (among other things) therapeutic combinations and compositions comprising a long-acting interleukin-15 (“IL-15”) receptor agonist and another pharmacologically active agent, i.e., an antibody such as a monoclonal antibody, and related methods of use, for example, in the treatment of conditions responsive to therapy effective to provide, for example, sustained immune activation and anti-tumor activity.

### BACKGROUND

Interleukin-15 (“IL-15”) is a pleiotropic cytokine that was first reported by Grabstein et al. (Grabstein et al. (1994) Science 264:965-968). Secreted as a 162-amino acid precursor, human IL-15 contains a 29-amino acid leader sequence and a 19-amino acid Pro-sequence; the mature protein is therefore 114 amino acids in length. Belonging to the four α-helix bundle family of cytokines, IL-15 binds to a heterotrimeric receptor, wherein a unique a subunit (IL-15Rα) confers receptor specificity to IL-15, and the β and γ subunits of this receptor share commonality with one or more other cytokine receptors. Giri et al. (1995) EMBO J. 14:3654-3663.

As a cytokine, IL-15 has effects on both the innate immune system and the adaptive immune system (DiSabitino et al. (2011) Cytokine Growth Factor Rev. 22:19-33). With respect to the innate immune system (which defends the host from foreign invaders generically), IL-15 causes the development of and maintains the survival of natural killer cells (“NK cells”) and natural killer-T cells (“NK T cells”), in addition to having other properties. Consistent with their role in the innate immune system, NK cells do not specifically attack the invading pathogen, rather, these cells destroy compromised host cells (such as tumor cells or virus-infected cells). NK T cells generate immunomodulatory cytokines, particularly interferon-γ, which result in a general activation of the immune response.

With respect to the adaptive immune system (which defends the host from a specific foreign invader following an initial encounter with that particular pathogen), IL-15 is necessary for the maintenance of the immunomodulatory cytokine-generating helper T cells. Importantly, IL-15 also supports the long-term maintenance of “antigen-experienced” memory T cells, which have the ability to rapidly reproduce, thereby generating a faster and stronger immune response upon re-exposure to the particular foreign pathogen invading the host.

Finally, notwithstanding its specific roles within both the innate and adaptive immune systems, IL-15 has significant and broad effects across both categories of immune systems. In particular, IL-15 inhibits or reduces apoptosis (or cell death) of a number of cells types (including dendritic cells, neutrophils, eosinophils, mast cells, CD4+ T cells, and B cells) associated within both categories of immune systems. IL-15-mediated responses have also been shown to have a role in the development, function, and survival of CD8+ T cells and intestinal intraepithelial lymphocytes.

Because it stimulates the proliferation and maintenance of many cells within the immune system that can fight against cells that appear to the host as foreign (or “non-self”), IL-15 has been proposed for use in the treatments of individuals suffering from cancer (Steel et al. (2012) Trends Pharmacol. Sci. 33(1):35-41). For example, an IL-15-based agonist has been proposed to treat myelomas (Wong et al. (2013) OncoImmunology 2(11), e26442:1-3). In addition, IL-15 pharmacotherapy has been proposed for treating individuals suffering from viral infections, such as HIV infection.

A long-acting IL-15 receptor agonist comprising at least one water-soluble polymer (e.g., polyethylene glycol) moiety stably covalently attached to an IL-15 amino group has been described (PCT Application No. PCT/US2018/032817, incorporated by reference herein in its entirety) as providing improved characteristics and in-vivo profiles, such as, for example, potent immune stimulatory effects, low systemic toxicity, stability and/or improved pharmacokinetics, improved therapeutic effects, among other improvements, in comparison to IL-15 and other IL-15 receptor agonists.

The generation and use of monoclonal antibodies (mAb) for treating certain cancers, including both hematological malignancies as well as solid tumors, has been established (Scott et al., 2012, Cancer Immunity, vol. 12, p. 14). The mechanism of action of tumor-associated mAbs includes one or more of the following: direct action on the tumor cell, immune-mediated action, and vascular and stromal ablation. Tumor-associated antigens targeted by mAbs include clusters of differentiation (CD) antigens (e.g., CD20, CD30, CD33, CD52), glycoproteins (e.g., EpCAM, CEA, gpA33, mucins, etc.), glycolipids (e.g., gangliosides such as GD2, GD3 and GM2), vascular targets (e.g., VEGF, VEGFR), growth factors (e.g., ErbB1/EGFR, ErbB2/HER2, ErbB3, c-MET, IGF1R), and stromal and extracellular matrix antigens (e.g., FAP, tenascin). A number of mAbs have been approved by the U.S. FDA for use in oncology (e.g., rituximab, ofatumumab, ZEVALIN®, BEXXAR®, gemtuzumab ozogamicin, brentuximab vedotin, cetuximab, and panitumumab), although certain types of cancer cells are more vulnerable than others to monoclonal antibody-based therapies.

Notwithstanding the foregoing approaches, however, there remains a need for improved anticancer immunotherapies. The present disclosure addresses these and other needs by providing, in particular, a combination therapy comprising a long-acting IL-15 receptor agonist and at least one mAb directed against a tumor antigen (the combination having a number of advantageous features to be described in greater detail below), as well as compositions and kits comprising such combinations, as well as related methods of preparation and use, which are believed to be new and completely unsuggested by the art.

### SUMMARY

In a first aspect, a method of treating a subject having cancer is provided herein. In particular, the method comprises administering to a subject a long-acting IL-15 receptor agonist and (b) a monoclonal antibody, such as a monoclonal antibody that targets, i.e., binds to, tumor cells, wherein steps (a) and (b) are carried out concurrently or sequentially and in any order. In some embodiments, the long-acting IL-15 receptor agonist has a structure:

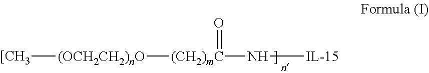

where the structure may also be depicted as [CH₃O(CH₂CH₂O)ₙ(CH₂)ₘC(O)NH]_{n′}-IL-15, wherein IL-15 is an interleukin-15 moiety, (n) is an integer from about 150 to about 3,000, (m) is an integer selected from 2, 3, 4, and 5, (n′) is 1, and ˜NH˜ represents an amino group of the IL-15 moiety; or is a pharmaceutically acceptable salt form thereof. In some particular embodiments, (m) in Formula (I) is 2 or 3. In a preferred embodiment, (m) in Formula (I) is 3. In some particular embodiments, (n) in Formula (I) has an average value of about 227, or an average value of about 340, or an average value of about 454, or an average value of about 681, or an average value of about 909. In one or more embodiments, (n) has a value of about 909.

In some embodiments, the antibody is an antibody that binds specifically to a tumor antigen selected from a phosphoprotein, a transmembrane protein, a glycoprotein, a glycolipid, and a growth factor.

In further embodiments of the method, the subject has a solid cancer. In some embodiments, the solid cancer is selected from the group consisting of breast cancer, ovarian cancer, colon cancer, colorectal cancer, gastric cancer, malignant melanoma, multiple myeloma, liver cancer, lymphoma, small cell lung cancer, non-small cell lung cancer, thyroid cancers, kidney cancer, cancer of the bile duct, brain cancer, cervical cancer, maxillary sinus cancer, bladder cancer, esophageal cancer, Hodgkin's disease and adrenocortical cancer, including metastatic forms of any of the foregoing.

In some other embodiments, the subject has lymphoma or leukemia.

In some further embodiments, the subject has multiple myeloma.

In some embodiments of the method, step (a) is carried out prior to step (b). In other embodiments, step (b) is carried out prior to step (a). In yet further embodiments, step (a) and step (b) are carried out concurrently or substantially concurrently. The method may further comprise one or more additional cycles of dosing of either or both of the long-acting IL-15 receptor agonist and the monoclonal antibody.

In some embodiments, the administering is effective to stimulate NK activation and proliferation to an extent greater than observed when the long-acting IL-15 receptor agonist is administered as a single agent, as measured in a suitable animal model. In some additional embodiments, the administering is effective to support CD8 T-cell survival and memory formation to an extent greater than observed when the long-acting IL-15 receptor agonist is administered as a single agent, as measured in a suitable animal model. In some further embodiments, the administering is effective to result in a decrease in the number of tumor cells that is greater than that observed upon administration of the long-acting receptor agonist administered as a single agent (i.e., as a monotherapy) and is greater than that observed upon administration of the monoclonal antibody as a single agent, as measured in a suitable animal model (examples of which are provided herein). In some related embodiments, the administering results in a 3-fold or greater reduction, or more preferably a 5-fold or greater reduction, or more preferably a 7-fold or greater reduction, in the number of tumor cells in the subject when compared to administration of an equivalent dose of the long-acting receptor agonist as a single agent. In some further embodiments, the administering is effective to induce proliferation of NK cells (i.e., to increase the number of NK cells) and to activate their tumor cell killing capability, e.g., in bone marrow tissue.

In some embodiments, the long-acting IL-15 receptor agonist is administered subcutaneously. In additional embodiments, the monoclonal antibody is administered intravenously.

In one or more embodiments, the antibody is a targeted monoclonal antibody that utilizes an antibody-dependent cellular toxicity (ADCC, also referred to as antibody-dependent cell-mediated cytotoxicity) mechanism of action.

In some embodiments, the monoclonal antibody is selected from an anti-CD19 antibody, an anti-CD20 antibody, and an anti-CD38 antibody. In further embodiments, the monoclonal antibody that binds specifically to a glycoprotein is selected from an anti-SLAMF7 antibody, an anti-EpCAM antibody, an anti-gpA3 antibody 3, and an anti-FBP antibody. In additional embodiments, the monoclonal antibody that binds specifically to a growth factor is selected from an anti-VEGF antibody, an anti-VEGFR antibody, and an anti-EGFR antibody. In yet some additional embodiments, the antibody is an anti-BCMA antibody. In some additional embodiments, the antibody is a multiple myeloma-targeted antibody.

In a second aspect, a therapeutic combination for use in treating a condition such as cancer is provided herein. The combination comprises a long-acting IL-15 receptor agonist and a monoclonal antibody, such as a monoclonal antibody that targets, i.e., binds to, tumor cells, including but not limited to the monoclonal antibodies as described herein.

In some related and more particular embodiments, the long-acting IL-15 receptor agonist has a structure:

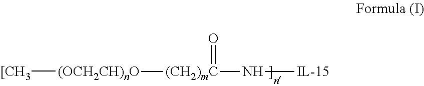

wherein IL-15 is an interleukin-15 moiety, (n) is an integer from about 150 to about 3,000, (m) is an integer selected from 2, 3, 4, and 5, (n′) is 1, and ˜NH˜ represents an amino group of the IL-15 moiety; or a pharmaceutically acceptable salt form thereof. In yet some additional embodiments, the monoclonal antibody is an antibody that binds specifically to a tumor antigen selected from a phosphoprotein, a transmembrane protein, a glycoprotein, a glycolipid, and a growth factor. In some embodiments, (m) in Formula (I) is 2 or 3. In some particular embodiments, (m) in Formula (I) is 3. In some further embodiments, (n) in Formula (I) has a value of about 227, or about 340, or about 454, or about 681, or about 909. In one or more embodiments, (n) has a value of about 909.

In a third aspect, a kit is provided herein. In embodiments, the kit comprises a therapeutic combination of a long-acting IL-15 receptor agonist and a monoclonal antibody as described herein, accompanied by instructions for use, wherein the long-acting IL-15 receptor agonist and the monoclonal antibody are each contained in one or more individual unit dosage forms. The kit and therapeutic combination are useful, for example, for treating a subject with cancer.

Additional aspects and embodiments are set forth in the following description and claims. The embodiments as described herein are meant to apply equally to each of the aspects described herein and are to be considered both singly and in combination as applicable, unless indicated otherwise.

### BRIEF DESCRIPTION OF THE DRAWINGS

FIG. 1 provides the amino acid sequence of an exemplary recombinant human IL-15 from E. coli (SEQ ID NO:1), a single, non-glycosylated polypeptide chain containing 115 amino acids, with a molecular weight of 12.9 kDa.

FIG. 2 is a graph illustrating the percent survival of mice inoculated with Daudi B lymphoma cells and treated with the following: (i) isotype control, (ii) rituximab at 40 mg/kg, (iii) a long-acting IL-15 receptor agonist at 0.3 mg/kg, or (iv) a combination of rituximab at 40 mg/kg and a long-acting IL-15 receptor agonist at 0.3 mg/kg as described in detail in Example 1.

FIG. 3 is a graph illustrating NK cell counts in bone marrow tissue following treatment of mice inoculated with Daudi B cells with the following: (i) daratumumab (0.5 mg/kg IP, 14 days following Daudi cell inoculation) and two doses of a long-acting IL-15 receptor agonist, i.e., mono(methoxyPEG-N-butanamide)interleukin-15 (0.3 mg/kg SC, one dose at each of 14 and 21 days after inoculation), (ii) single agent daratumumab (0.5 mg/kg IP, 14 days following Daudi cell inoculation), (iii) single agent mono(methoxyPEG-N-butanamide)interleukin-15 (0.3 mg/kg SC, 14 and 21 days after inoculation) when compared to (iv) an untreated control group, as described in Example 2.

FIG. 4 is a graph illustrating the numbers of Daudi B cells in bone marrow tissue following treatment of mice inoculated with Daudi B cells with the following: (i) daratumumab (0.5 mg/kg IP, 14 days following Daudi cell inoculation) and two doses of mono(methoxyPEG-N-butanamide)interleukin-15 (0.3 mg/kg SC, one dose at each of 14 and 21 days after inoculation), (ii) single agent daratumumab (0.5 mg/kg IP, 14 days following Daudi cell inoculation), (iii) single agent mono(methoxyPEG-N-butanamide)interleukin-15 (0.3 mg/kg SC, 14 and 21 days after inoculation) when compared to (iv) an untreated control group, as described in Example 2.

FIG. 5 is a graph illustrating the percent survival of mice inoculated with Daudi B cell lymphoma cells, followed by treatment with the following: (i) isotype control, (ii) daratumumab at 0.5 mg/kg IP (iii) a long-acting IL-15 receptor agonist, mono(methoxyPEG-N-butanamide)interleukin-15, at 0.3 mg/kg SC, or (iv) a combination of the long-acting IL-15 receptor agonist at 0.3 mg/kg and daratumumab at 0.5 mg/kg, SC as described in detail in Example 3.

FIG. 6 is a graph illustrating granzyme B induction in bone marrow NK cells following treatment of mice inoculated with Daudi B cells with: (i) daratumumab (0.5 mg/kg IP, 14 days following Daudi cell inoculation) and two doses of mono(methoxyPEG-N-butanamide)interleukin-15 (0.3 mg/kg SC, one dose at each of 14 and 21 days after inoculation, (ii) single agent daratumumab (0.5 mg/kg IP, 14 days following Daudi cell inoculation), (iii) single agent mono(methoxyPEG-N-butanamide)interleukin-15 (0.3 mg/kg SC, 14 and 21 days after inoculation), when compared to (iv) an untreated control group, as described in detail in Example 2.

FIGS. 7A and 7B are graphs illustrating the fraction of NK cells in the bone marrow compartment expressing NKG2A (FIG. 7A) or NKG2D (FIG. 7B) on the cell surface following treatment of mice inoculated with Daudi B cells with: (i) daratumumab (0.5 mg/kg IP, 14 days following Daudi cell inoculation) and two doses of Conjugate 1 (0.3 mg/kg or 0.03 mg/kg SC, one dose at each of 14 and 21 days after inoculation, (ii) single agent daratumumab (0.5 mg/kg IP, 14 days following Daudi cell inoculation), (iii) single agent Compound 1 (0.3 mg/kg SC, 14 and 21 days after inoculation), when compared to (iv) an untreated control group or (v) an isotype control (0.5 mg/kg), as described in detail in Example 4.

FIGS. 8A-8D are histograms illustrating % proliferation of human NK cells within a human peripheral blood mononuclear cell (PBMC) preparation with (i) Compound 1 alone (FIG. 8C), (ii) hIgG alone (FIG. 8B), (iii) Compound 1+hIgG (FIG. 8D), or (iv) an untreated control (FIG. 8A), as described in detail in Example 5.

FIG. 9A is a graph illustrating the median fluorescence intensity (MFI) signal for CD69 detecting antibodies on the surface of CD56+ NK cells within a PBMC preparation cultured overnight with (i) a hIgG coated plate, (ii) Compound 1 (1 μg/ml), (iii) a hIgG coated plate+Compound 1 (1 μg/ml), or (ii) a control showing NK cell activation, as described in detail in Example 6.

FIG. 9B is a graph illustrating the median fluorescence intensity (MFI) signal for CD107a detecting antibodies on the surface of CD56+ NK cells within a PBMC preparation cultured overnight with (i) a hIgG coated plate, (ii) Compound 1 (1 μg/ml), (iii) a hIgG coated plate+Compound 1 (1 μg/ml), or (ii) a control showing NK cell activation, as described in detail in Example 6.

FIG. 9C is a graph illustrating granzyme B induction in human PBMCs following exposure to: (i) a hIgG coated plate, (ii) Compound 1 (1 μg/ml), a hIgG coated plate+Compound 1 (1 μg/ml), or (ii) a control, as described in detail in Example 6. The graph illustrates the secreted Granzyme B concentration in μg/ml for each of the four treatments.

FIG. 10A is a graph illustrating the pSTAT5 percent positivity within KHYG-1 cells after incubation with IL-15 or a long-acting IL-15 receptor agonist (mono(methoxyPEG-N-butanamide)interleukin-15), as described in detail in Example 7.

FIG. 10B is a graph of % maximal proliferation of KHYG-1 cells after incubation with IL-15 or a long-acting IL-15 receptor agonist (mono(methoxyPEG-N-butanamide)interleukin-15), as described in detail in Example 7.

FIG. 11 is a graph illustrating the % maximal proliferation of CD56+ human NK cells after incubation with IL-15 or a long-acting IL-15 receptor Agonist (mono(methoxyPEG-N-butanamide)interleukin-15), as described in detail in Example 8.

FIG. 12 is a graph illustrating the %7-AAD+ target cells for human multiple myeloma cells pre-coated with daratumumab (+) after stimulation with a long-acting IL-15 receptor agonist (mono(methoxyPEG-N-butanamide)interleukin-15) (+) or untreated (−), as described in detail in Example 9.

FIG. 13A is a graph illustrating the fraction of NK cells in the bone marrow compartment expressing CD16 (FIG. 13A) on the cell surface (% of cells expressing CD16 in the total NK cells) following treatment of mice inoculated with Daudi B cells with: (i) daratumumab (0.5 mg/kg IP, 14 days following Daudi cell inoculation) and two doses of Conjugate 1 (0.3 mg/kg or 0.03 mg/kg SC, one dose at each of 14 and 21 days after inoculation, (ii) single agent daratumumab (0.5 mg/kg IP, 14 days following Daudi cell inoculation), (iii) single agent Compound 1 (0.3 mg/kg SC, 14 and 21 days after inoculation), when compared to (iv) an untreated control group or (v) an isotype control (0.5 mg/kg), as described in detail in Example 10.

FIG. 13B is a graph illustrating the CD16 expression change (increase in Compound 1 treated groups) on a per cell basis for CD16+ bone marrow NK cells as measured by median fluorescence intensity (MFI) signal following treatment of mice inoculated with Daudi B cells with: (i) daratumumab (0.5 mg/kg IP, 14 days following Daudi cell inoculation) and two doses of Conjugate 1 (0.3 mg/kg or 0.03 mg/kg SC, one dose at each of 14 and 21 days after inoculation, (ii) single agent daratumumab (0.5 mg/kg IP, 14 days following Daudi cell inoculation), (iii) single agent Compound 1 (0.3 mg/kg SC, 14 and 21 days after inoculation), when compared to (iv) an untreated control group or (v) an isotype control (0.5 mg/kg), as described in detail in Example 10.

FIG. 14 is a graph illustrating the granzyme B expression in individual bone marrow NK cells as measured by median fluorescence intensity (MFI) signal following treatment of mice inoculated with Daudi B cells with: (i) daratumumab (0.5 mg/kg IP, 14 days following Daudi cell inoculation) and two doses of Compound 1 (0.3 mg/kg or 0.03 mg/kg SC, one dose at each of 14 and 21 days after inoculation, (ii) single agent daratumumab (0.5 mg/kg IP, 14 days following Daudi cell inoculation), (iii) single agent Compound 1 (0.3 mg/kg SC, 14 and 21 days after inoculation), when compared to (iv) an untreated control group or (v) an isotype control (0.5 mg/kg), as described in detail in Example 11.

FIGS. 15A and 15B are graphs illustrating %7-AAD+ target cells for human multiple myeloma cells pre-coated with daratumumab (+) (FIG. 15A) or rituximab (+) (FIG. 15B) after stimulation with a long-acting IL-15 receptor agonist (mono(methoxyPEG-N-butanamide)interleukin-15) (+) or untreated (−), as described in detail in Example 12.

FIG. 16 is a graph illustrating the percent survival of SCID or SCID beige mice inoculated with Daudi B cell lymphoma cells, followed by treatment with the following: (i) untreated control for SCID (□), (ii) untreated control for SCID beige (∘) mice, (iii) a combination of the long-acting IL-15 receptor agonist at 0.3 mg/kg and daratumumab at 0.5 mg/kg, SC for SCID mice (▪); and (iv) a combination of the long-acting IL-15 receptor agonist at 0.3 mg/kg and daratumumab at 0.5 mg/kg, SC for SCID beige mice (●) as described in detail in Example 13.

FIG. 17A is a graph illustrating Daudi cell counts in bone marrow tissue following treatment of mice inoculated with Daudi B cells with the following: (i) high dose daratumumab (5 mg/kg IP, 14 days following Daudi cell inoculation) and two low doses of a long-acting IL-15 receptor agonist, i.e., mono(methoxyPEG-N-butanamide)interleukin-15 (compound 1) (0.03 mg/kg IV, one dose at each of 14 and 21 days after inoculation) (♦), (ii) single agent high dose daratumumab (5 mg/kg IP, 14 days following Daudi cell inoculation) (▾), (iii) single agent low dose mono(methoxyPEG-N-butanamide)interleukin-15 (compound 1) (0.03 mg/kg IV, 14 and 21 days after inoculation) (▴) when compared to (iv) an untreated control group (●), as described in detail in Example 14. FIG. 17B is a graph illustrating Daudi cell counts in bone marrow tissue following treatment of mice inoculated with Daudi B cells with the following: (i) low dose daratumumab (0.05 mg/kg IP, 14 days following Daudi cell inoculation) and two high doses of a long-acting IL-15 receptor agonist, i.e., mono(methoxyPEG-N-butanamide)interleukin-15 (compound 1) (0.6 mg/kg IV, one dose at each of 14 and 21 days after inoculation) (♦), (ii) single agent low dose daratumumab (0.05 mg/kg IP, 14 days following Daudi cell inoculation) (▾), (iii) single agent high dose mono(methoxyPEG-N-butanamide)interleukin-15 (compound 1) (0.6 mg/kg IV, 14 and 21 days after inoculation) (▴) when compared to (iv) an untreated control group (●), as described in detail in Example 14.

FIG. 18A is a graph illustrating the intratumoral fraction of NK cells at day 3 or day 5 following treatment of mice bearing subcutaneous HCT-116 colon colorectal cell tumors with cetuximab (20 mg/kg, IP) and Conjugate 1 (0.3 mg/kg IV) in comparison to a vehicle control, as described in detail in Example 15.

FIG. 18B is a graph illustrating intratumoral NK cell counts at day 3 or day 5 following treatment of mice bearing subcutaneous HCT-116 tumors with cetuximab (20 mg/kg, IP) and Conjugate 1 (0.3 mg/kg IV) in comparison to a vehicle control, as described in detail in Example 15.

FIG. 18C is a graph illustrating NK cell proliferation as shown by % Ki67 positivity in blood or tumor cells following treatment of mice bearing subcutaneous HCT-116 tumors with cetuximab (20 mg/kg, IP) and Conjugate 1 (0.3 mg/kg IV) in comparison to a vehicle control, as described in detail in Example 15.

FIG. 18D is a graph illustrating Granzyme B expression (% GzmB+) in blood or tumor cells following treatment of mice bearing subcutaneous HCT-116 tumors with cetuximab (20 mg/kg, IP) and Conjugate 1 (0.3 mg/kg IV) in comparison to a vehicle control, as described in detail in Example 15.

FIG. 18E is a graph illustrating the CD16 cell surface expression on NK cells as measured by median fluorescence intensity (MFI) signal following treatment of mice bearing subcutaneous HCT-116 tumors with cetuximab (20 mg/kg, IP) and Conjugate 1 (0.3 mg/kg IV) in comparison to a vehicle control, as described in detail in Example 15.

FIG. 18F is a graph illustrating the intratumoral fraction of NK cells expressing NKG2D (% NKG2D+) on the cell surface following treatment of mice bearing subcutaneous HCT-116 tumors with cetuximab (20 mg/kg, IP) and Conjugate 1 (0.3 mg/kg IV) in comparison to a vehicle control, as described in detail in Example 15.

FIG. 19A is a graph illustrating the intratumoral fraction of NK cells at day 3 or day 5 following treatment of mice bearing subcutaneous FaDu squamous cell carcinoma tumors with cetuximab (20 mg/kg, IP) and Conjugate 1 (0.3 mg/kg IV) in comparison to a vehicle control, as described in detail in Example 16.

FIG. 19B is a graph illustrating intratumoral NK cell counts at day 3 or day 5 following treatment of mice bearing subcutaneous FaDu tumors with cetuximab (20 mg/kg, IP) and Conjugate 1 (0.3 mg/kg IV) in comparison to a vehicle control, as described in detail in Example 16.

FIG. 19C is a graph illustrating NK cell proliferation as shown by % Ki67 positivity in blood or tumor cells following treatment of mice bearing subcutaneous FaDu tumors with cetuximab (20 mg/kg, IP) and Conjugate 1 (0.3 mg/kg IV) in comparison to a vehicle control, as described in detail in Example 16.

FIG. 19D is a graph illustrating Granzyme B expression (% GzmB+) in blood or tumor cells following treatment of mice bearing subcutaneous FaDu tumors with cetuximab (20 mg/kg, IP) and Conjugate 1 (0.3 mg/kg IV) in comparison to a vehicle control, as described in detail in Example 16.

FIG. 19E is a graph illustrating the CD16 cell surface expression on NK cells as measured by median fluorescence intensity (MFI) signal following treatment of mice bearing subcutaneous FaDu tumors with cetuximab (20 mg/kg, IP) and Conjugate 1 (0.3 mg/kg IV) in comparison to a vehicle control, as described in detail in Example 16.

FIG. 19F is a graph illustrating the intratumoral fraction of NK cells expressing NKG2D (% NKG2D+) on the cell surface following treatment of mice bearing subcutaneous FaDu tumors with cetuximab (20 mg/kg, IP) and Conjugate 1 (0.3 mg/kg IV) in comparison to a vehicle control, as described in detail in Example 16.

FIG. 20 is a graph illustrating the relative tumor volume for 0-27 days following treatment of mice inoculated with H1975 lung carcinoma cells with the following: (i) cetuximab (0.25 mg/kg, IP) administered on day 9, 12, and 16 after tumor inoculation and Compound 1(0.3 mg/kg, IV) administered on day 9, 16 and 23 days after inoculation (Δ), (ii) single agent cetuximab (0.25 mg/kg, IP, BIW×3) (▴), (iii) single agent Compound 1 (0.3 mg/kg, IV, q7d×3) (▾), in comparison to a vehicle control (●), as described in detail in Example 17.

FIG. 21A is a graph illustrating the relative tumor volume for 0-21 days following treatment of mice inoculated with HT-29 colorectal carcinoma cells with the following: (i) cetuximab (40 mg/kg, IP, BIW×3) and Compound 1(0.3 mg/kg, IV, q7d×3) (Δ), (ii) single agent cetuximab (40 mg/kg, IP, BIW×3) (▴), (iii) single agent Compound 1 (0.3 mg/kg, IV, q7d×3) (▾), in comparison to a vehicle control (●), as described in detail in Example 18.

FIG. 21B is a graph illustrating the tumor growth delay (TVQT) as percent survival of mice inoculated with HT-29 colorectal carcinoma cells and treated with the following: (i) cetuximab (40 mg/kg, IP, BIW×3) and Compound 1(0.3 mg/kg, IV, q7d×3) (▾), (ii) single agent cetuximab (40 mg/kg, IP, BIW×3) (▪), (iii) single agent Compound 1 (0.3 mg/kg, IV, q7d×3) (▴), in comparison to a vehicle control (●) for 0-23 days after treatment start (mean tumor volume of ˜150 mm³), as described in detail in Example 18.

FIG. 22A is a graph illustrating the relative tumor volume for 0-19 days following treatment of mice inoculated with HCT-116 colorectal carcinoma cells with the following: (i) cetuximab (40 mg/kg, IP, BIW×3) and Compound 1(0.3 mg/kg, IV, q7d×3) (Δ), (ii) single agent cetuximab (40 mg/kg, IP, BIW×3) (▴), (iii) single agent Compound 1 (0.3 mg/kg, IV, q7d×3) (▾), in comparison to a PBS vehicle control (OP, BIW×3) (●), as described in detail in Example 19.

FIG. 22B is a graph illustrating the tumor growth delay (TVQT) as percent survival of mice inoculated with HCT-116 colorectal carcinoma cells and treated with the following: (i) cetuximab (40 mg/kg, IP, BIW×3) and Compound 1(0.3 mg/kg, IV, q7d×3) (Δ), (ii) single agent cetuximab (40 mg/kg, IP, BIW×3) (▴), (iii) single agent Compound 1 (0.3 mg/kg, IV, q7d×3) (▾), in comparison to a PBS vehicle control (IP, BIW×3) (●), as described in detail in Example 19.

FIG. 23A is a graph illustrating the % CD45-EpCAM+7-AAD+ target cells for HCT-116 colorectal carcinoma cells pre-coated with cetuximab (+) alone or after stimulation with Compound 1 (+), with an isotype control (+), or untreated (−), as described in detail in Example 20.

FIG. 23B is a graph illustrating the % CD45-7-AAD+ target cells for FaDu squamous cell carcinoma cells (HNSCC) pre-coated with cetuximab (+) alone or after stimulation with Compound 1 (+), with an isotype control (+), or untreated (−), as described in detail in Example 20.

FIG. 24 is a graph illustrating the relative tumor volume for 0-35 days following treatment of mice inoculated with SKOV-3 ovarian adenocarcinoma cells with the following: (i) trastuzumab (13.5 mg/kg, IV, BIW×3) and Compound 1 (0.3 mg/kg, IV, q7d×3) (Δ), (ii) single agent trastuzumab (13.5 mg/kg, IV, BIW×3) (▴), (iii) single agent Compound 1 (0.3 mg/kg, IV, q7d×3) (▾), in comparison to a vehicle control (●), as described in detail in Example 21.

FIG. 25 is a graph illustrating the relative tumor volume for 0-35 days following treatment of mice inoculated with NCI-N87 gastric carcinoma cells with the following: (i) trastuzumab (3/1/1 mg/kg, IV, q7d×3) and Compound 1 (0.3 mg/kg, IV, q7d×3) (Δ), (ii) single agent trastuzumab (3/1/1 mg/kg, IV, q7d×3) (▴), (iii) single agent Compound 1 (0.3 mg/kg, IV, q7d×3) (▾), in comparison to a vehicle control (●), as described in detail in Example 22.

### DETAILED DESCRIPTION

Before describing one or more aspects or embodiments of the present disclosure in detail, it should be noted that the presented disclosure is not intended to be limited to the particular synthetic techniques, IL-15 moieties, and the like, as such may vary as would be understood by one having ordinary skill in the art to which this disclosure applies.

In describing and claiming certain features of this disclosure, the following terminology will be used in accordance with the definitions described below unless indicated otherwise.

As used in this specification, the singular forms “a,” “an,” and “the” include plural referents unless the context clearly dictates otherwise.

“Water soluble, non peptidic polymer” refers to a polymer that is at least 35% (by weight) soluble, preferably greater than 70% (by weight), and more preferably greater than 95% (by weight) soluble, in water at room temperature. Typically, an unfiltered aqueous preparation of a “water soluble” polymer transmits at least 75%, more preferably at least 95%, of the amount of light transmitted by the same solution after filtering. It is most preferred, however, that the water-soluble polymer is at least 95% (by weight) soluble in water or completely soluble in water. With respect to being “non-peptidic,” a polymer is non-peptidic when it has less than 35% (by weight) of amino acid residues.

“PEG” or “polyethylene glycol,” as used herein, is meant to encompass any water-soluble poly(ethylene oxide). Unless otherwise indicated, a “PEG polymer” or a polyethylene glycol is one in which substantially all (preferably all) monomeric subunits are ethylene oxide subunits, though, the polymer may contain distinct end capping moieties or functional groups, e.g., for conjugation. PEG polymers for use in the present disclosure will comprise one of the two following structures: “—(CH₂CH₂O)ₙ—” or “—(CH₂CH₂O)ₙ₋₁CH₂CH₂—,” depending upon whether or not the terminal oxygen(s) has been displaced, e.g., during a synthetic transformation. As stated above, for PEG polymers, the variable (n) may range from about 3 to 4000, and the terminal groups and architecture of the overall PEG can vary. Exemplary or preferred PEG-comprising molecules may however comprise one or more particular PEG architectures and/or linkers, and/or molecular weight ranges.

Molecular weight in the context of a water-soluble polymer, such as PEG, can be expressed as either a number average molecular weight or a weight average molecular weight. Unless otherwise indicated, all references to molecular weight herein refer to the weight average molecular weight. Both molecular weight determinations, number average and weight average, can be measured using gel permeation chromatography or other liquid chromatography techniques (e.g. gel filtration chromatography). Most commonly employed are gel permeation chromatography and gel filtration chromatography. Other methods for determining molecular weight include end-group analysis or the measurement of colligative properties (e.g., freezing-point depression, boiling-point elevation, or osmotic pressure) to determine number average molecular weight or the use of light scattering techniques, ultracentrifugation, MALDI TOF, or viscometry to determine weight average molecular weight. PEG polymers are typically polydisperse (i.e., the number average molecular weight and the weight average molecular weight of the polymers are not equal), possessing low polydispersity values of preferably less than about 1.2, more preferably less than about 1.15, still more preferably less than about 1.10, yet still more preferably less than about 1.05, and most preferably less than about 1.03.

A “physiologically cleavable” or “hydrolyzable” or “degradable” bond is a relatively labile bond that reacts with water (i.e., is hydrolyzed) under physiological conditions. The tendency of a bond to hydrolyze in water may depend not only on the general type of linkage connecting two atoms within a given molecule but also on the substituents attached to these atoms. Appropriate hydrolytically unstable or weak linkages may include but are not limited to carboxylate ester, phosphate ester, anhydrides, acetals, ketals, acyloxyalkyl ether, imines, orthoesters, peptides, oligonucleotides, thioesters, and carbonates.

A covalent “releasable” linkage, for example, in the context of a polyethylene glycol that may be covalently attached to an active moiety such as interleukin-15, is one that releases or detaches a polyethylene glycol polymer from the active moiety under physiological conditions, e.g., by any suitable mechanism, at a rate that is clinically useful and includes, for example and without limitation, hydrolyzable bonds and enzymatically degradable linkages.

An “enzymatically degradable linkage” means a linkage that is subject to degradation by one or more enzymes.

A “stable” linkage or bond refers to a chemical bond that is substantially stable in water, that is to say, does not undergo hydrolysis under physiological conditions to any appreciable extent over an extended period of time. Examples of hydrolytically stable linkages generally include but are not limited to the following: carbon-carbon bonds (e.g., in aliphatic chains), ethers, amides, amines, and the like. Generally, a stable linkage is one that exhibits a rate of hydrolysis of less than about 1-2% per day under physiological conditions. Hydrolysis rates of representative chemical bonds can be found in most standard chemistry textbooks.

“Substantially” or “essentially” means nearly totally or completely, for instance, 95% or greater of a given quantity.

Similarly, “about” or “approximately” as used herein means within plus or minus 5% of a given quantity.

“Optional” or “optionally” means that the subsequently described circumstance may but need not necessarily occur, so that the description includes instances where the circumstance occurs and instances where it does not.

“Pharmaceutically acceptable excipient” or “pharmaceutically acceptable carrier” refers to a component that may be included in a composition as described herein and causes no significant adverse toxicological effects to a subject.

The phrases “pharmaceutically effective amount” and “pharmacologically effective amount” and “therapeutically effective amount” and “physiologically effective amount” are used interchangeably herein and refer to the amount of a long-acting IL-15 receptor agonist as provided herein or the amount of a monoclonal antibody as described herein that is needed to provide a desired level of the substance in the bloodstream or in a target tissue to produce a desired biological or medicinal response. For example, such a response may be to destroy target cancer cells and/or to slow or arrest the progression of cancer in a subject, and/or to increase the number of NK cells of a patient. The term also applies to a dose that will induce a particular response in target cells. The precise amount will depend upon numerous factors, such as for example, the particular condition being treated, the intended patient population, individual patient considerations, the components and physical characteristics of the therapeutic composition and particular combination to be administered, and the like, and may be readily determined by one skilled in the art.

The term “IL-15 moiety,” as used herein, refers to a peptide or protein moiety having human IL-15 activity. In addition, the term “IL-15 moiety” encompasses both the IL-15 moiety prior to conjugation as well as the IL-15 moiety residue following conjugation. As will be explained in further detail below, one of ordinary skill in the art can determine whether any given moiety has IL-15 activity. Proteins comprising an amino acid sequence corresponding to any one of SEQ ID NOs: 1 through 3 is an IL-15 moiety, as well as any protein or polypeptide substantially homologous thereto. As used herein, the term “IL-15 moiety” includes such peptides and proteins modified deliberately, as for example, by site directed mutagenesis or accidentally through mutations. These terms also include analogs having from 1 to 6 additional glycosylation sites, analogs having at least one additional amino acid at the carboxy terminal end of the peptide or protein wherein the additional amino acid(s) includes at least one glycosylation site, and analogs having an amino acid sequence which includes at least one glycosylation site. The term includes naturally, recombinantly and synthetically produced IL-15 moieties. The IL-15 moiety may be produced by any suitable method as known in the art. In embodiments, the IL-15 moiety is recombinantly produced in an E. coli or a Chinese hamster ovary (CHO) expression system. Reference to a long-acting IL-15 receptor agonist as described herein is meant to encompass pharmaceutically acceptable salt forms thereof.

An “antibody” as used herein, is meant in a broad sense and includes glycoproteins belonging to the immunoglobulin (Ig) superfamily. Antibodies are intended to include polyclonal antibodies, monoclonal antibodies (e.g. murine, human, human-adapted, humanized and chimeric), antibody fragments, and single chain antibodies that bind specifically to an antigen (e.g. a tumor antigen). The fragment, antigen-binding (Fab) region comprises a constant domain and at least one variable domain from each of the heavy and light chains. The antibodies as described herein at least have antibody dependent cellular cytotoxicity (ADCC) as a mechanism of action.

The term “monoclonal antibody” (mAb) as used herein refers to non-naturally occurring antibody molecules obtained from a population of substantially homogeneous molecules such that the antibody molecules have primary sequences are essentially identical, excepting naturally occurring mutations that may be present in minor amounts. Monoclonal antibodies are highly specific to a single binding site or a particular epitope. A monoclonal antibody is an example of an isolated antibody. Monoclonal antibodies may be produced by any means as known in the art including, without limitation, hybridoma culture techniques, recombinant methods, and transgenic methods.

It should be understood the target binding sequence of the antibody may be altered or modified to improve affinity for the target, to humanize the target-binding sequence, to improve its production in cell culture, to reduce its immunogenicity in vivo, to create a multispecific antibody, among others. Such altered antibodies are specifically contemplated herein.

An “isolated” antibody is one that has been identified and separated and/or recovered from a component of its natural environment.

“Binding affinity” as used herein refers to the strength of the interaction between an antibody's antigen binding site and its binding partner (e.g., an antigen). The affinity of an antibody for its antigen can generally be represented by the affinity constant (K_{A}), the amount of antibody-antigen complex at equilibrium, or the equilibrium dissociation constant (K_{D}). Affinity can be measured by any method as known in the art including, but not limited to, ELISAs, gel-shift assays, pull-down assays, equilibrium dialysis, analytical ultracentrifugation, surface plasmon resonance (SPR), isothermal titration calorimetry (ITC), and spectroscopic assays.

The term “patient,” or “subject” as used herein refers to a living organism suffering from or prone to a condition that can be prevented or treated by administration of a compound or composition as provided herein. Subjects include, but are not limited to, mammals (e.g., murines, simians, equines, bovines, porcines, canines, felines, and the like), and preferably are human.

The term “substantially homologous” or “substantially identical” means that a particular subject sequence, for example, a mutant sequence, varies from a reference sequence by one or more substitutions, deletions, or additions, the net effect of which does not result in an adverse functional dissimilarity between the reference and subject sequences. For purposes herein, a sequence having greater than 95 percent homology (identity), equivalent biological activity (although not necessarily equivalent strength of biological activity), and equivalent expression characteristics to a given sequence is considered to be substantially homologous (identical). For purposes of determining homology, truncation of the mature sequence should be disregarded. Exemplary IL-15 polypeptides for use herein include those sequences that are substantially homologous to SEQ ID NO: 1. SEQ ID NO:2 is nearly identical to SEQ ID NO:1, with the exception that SEQ ID NO:2 has a methionine at the beginning of the sequence that is required for initiating translation in E. coli.

The term “fragment” means any protein or polypeptide having the amino acid sequence of a portion or fragment of the protein or polypeptide, e.g. an IL-15 moiety, and having the biological activity, or substantially the biological activity, of the protein or polypeptide, e.g. IL-15. Fragments include proteins or polypeptides produced by proteolytic degradation as well as proteins or polypeptides produced by chemical synthesis by methods routine in the art.

Amino acid residues in peptides are abbreviated as follows: Phenylalanine is Phe or F; Leucine is Leu or L; Isoleucine is Ile or I; Methionine is Met or M; Valine is Val or V; Serine is Ser or S; Proline is Pro or P; Threonine is Thr or T; Alanine is Ala or A; Tyrosine is Tyr or Y; Histidine is His or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; and Glycine is Gly or G.

### Overview

The instant disclosure is directed to, among other things, providing combinations, compositions, methods, and kits relating to the treatment of conditions such as cancer, comprising (a) a long-acting IL-15 receptor agonist, and (b) an antibody directed against a tumor antigen, where the tumor-directed antibody includes antibody-dependent cellular cytotoxicity (ADCC) as a mechanism of action. Such compositions and methods will ideally possess several advantageous and unpredictable features, such as, for example, at least one, if not more, of the following: an increased tumor clearance; increased and/or long-term survival of the patient; increased activity of one or both of the long-acting IL-15 receptor agonist and the antibody when administered in combination, when compared to either active agent administered singly, especially within a target tissue compartment; enhanced degranulation of NK cells; increased NK cell proliferation; increased NK cell viability; increased T cell, e.g. CD8+ T cell, proliferation and/or increased T cell, e.g. CD8+ T cell, viability. Surprisingly, the Applicants have arrived at combination of a long-acting IL-15 receptor agonist and at least one antibody (such as a monoclonal antibody) directed against a tumor antigen that possesses a unique combination of advantageous properties, to be described in greater detail below and illustrated in the supporting examples.

The combination of a long-acting IL-15R agonist as described herein and targeted antibodies that mediate tumor killing by ADCC has been discovered to exhibit an enhanced immunotherapeutic effect. ADCC is a crucial mechanism in tumor depletion by certain tumor-targeted antibodies, wherein receptors on NK cells recognize the tumor cell-bound antibodies. Re-engagement of the NK cell receptor to the antibody triggers release of cytotoxic granules and/or cytokines to kill the tumor cells. It has been discovered that the long-acting IL-15R agonists described herein are effective to increase the efficacy of tumor-targeting antibody therapies with an ADCC mechanism of action by expanding NK cells with increased cytotoxicity and/or other functional activation.

### Therapeutic Combinations, Compositions and Methods of Use

In a first aspect, a method for treating a subject afflicted with a cancer or a tumor are described herein. The method comprises administering, together or separately, an antibody, or antigen-binding portion thereof, that binds specifically to a tumor antigen and a long-acting IL-15 receptor agonist Due to the ability of the long-acting IL-15 receptor agonist to induce proliferation of NK cells, and to activate their tumor-cell killing capability, a combination therapeutic approach has been developed in which a long-acting IL-15 receptor agonist such as mono(methoxyPEG-N-butanamide)interleukin-15 (also referred to as mono(mPEG-butanamide)interleukin-15, mono(mPEG-butanamide)IL-15, or mono-mPEG-SBA-IL15) is combined with a tumor-cell recognizing therapeutic monoclonal antibody to thereby generate an increased number of cell-killing activated NK cells that can also bind effectively to the antibody molecules, and may then be directed to the tumor cells by the antibodies to facilitate enhanced synergistic tumor cell killing. See, for example, the results described in the accompanying examples herein.

By way of clarity, with regard to the sequence of administering, wherein the term “administering” is used in this instance to refer to delivery of either the long-acting IL-15 receptor agonist or the tumor-directed antibody, the long-acting IL-15 receptor agonist and the tumor-directed antibody may be administered concurrently or sequentially and in any order. Moreover, treatment of either component of the combination may comprise a single cycle of therapy or may comprise multiple cycles. That is to say, following administration of the long-acting IL-15 agonist and administration of the tumor-directed antibody, additional rounds of therapy may include administration of the long acting IL-15 receptor agonist in combination with administration of the tumor-directed antibody, administration of the long-acting IL-15 receptor agonist without further administration of the tumor-directed antibody, or administration of the tumor-directed antibody without further administration of the long-acting IL-15 receptor agonist, or any combination of the above administrations.

In a second aspect, a composition (or compositions) comprising an antibody, or antigen-binding portion thereof, that binds specifically to a tumor antigen and a long-acting IL-15 receptor agonist is described herein.

Generally, the antibodies as described herein are directed against a protein expressed on the cell surface of a cancer or tumor cell, referred to hereafter as a cancer antigen or tumor antigen. A number of cancer or tumor antigens are known in the art. Non-limiting examples include phosphoproteins, transmembrane proteins, glycoproteins, glycolipids, and growth factors. Assays for determining whether a given compound can act as an antibody to any of the antigens or targets as described herein can be determined through routing experimentation by one of ordinary skill in the art.

In some embodiments, the antibody is an immunoglobulin G (IgG) type of antibody, typically found in human blood circulation.

In some embodiments, the antibody is an anti-CD16, anti-CD19, an anti-CD20, or an anti-CD38 antibody, that is, an antibody that specifically binds to CD16, CD19, CD20, CD30, CD38, or CD52.

The human CD19 antigen is a 95 kDa glycoprotein belonging to the immunoglobulin (Ig) superfamily. CD19 is a biomarker for normal and neoplastic B cells as well as for follicular dendritic cells. CD19 is expressed from early stages of pre-B cell development through terminal differentiation, regulating B lymphocyte development and function. Expression of CD19 is highly conserved on most B cell tumors including B cell lymphomas such as non-Hodgkin lymphoma. CD19 is also expressed in most types of leukemia including B cell leukemias, acute lymphoblastic leukemia (ALL), chronic lymphocytic leukemia (CLL), and Waldenstrom's Macroglobulinemia (WM). The majority of B cell malignances (lymphomas and leukemias) express CD19 at normal to high levels. In embodiments, the antibody is an anti-CD19 monoclonal antibody. Some exemplary anti-CD19 antibodies contemplated for use in the methods and compositions herein include, but are not limited to, and for example, an anti-B4-bR, BiTE (a bi-specific T-cell engaging antibody), MEDI-551 (MedImmune, LLC), MOR-208 (MorphoSys AG), blinatumomab, a bi-specific anti-CD19/CD3 BiTE® antibody, (Blincyto®, Amgen), coltuximab ravtansine (ImmunoGen Inc. and Sanofi), denintuzumab mafodotin (Seattle Genetics), taplitumomab paptox (National Cancer Institute), XmAb 5871 (Amgen and Xencor Inc.), MDX-1342 (Medarex), AFM11 (Affimed Therapeutics), and the anti-CD19 antibody described in U.S. Pat. No. 8,691,952 (huB4, DI B4, Merck). In one or more embodiments, the combination of long-acting IL-15 receptor agonist and anti-CD19 antibody are used in the treatment of B cell malignancies including, without limitation, non-Hodgkin lymphoma, acute lymphoblastic leukemia (ALL), chronic lymphocytic leukemias (CLL), and Waldenstrom's Macroglobulinemia (WM).

CD20 is a non-glycosylated phosphoprotein of approximately 33-37 kD that is expressed on the surface of almost all normal and malignant B cells. CD20 mAbs can exert an anti-tumor effect via Fab-mediated effects that involve the activation of effector mechanisms (Boross et al., Am J Cancer 2(6):676-690, 2012). In one or more embodiments, the antibody that is administered with a long-acting IL-15 receptor agonist as described herein is an anti-CD20 monoclonal antibody. Some exemplary anti-CD20 antibodies contemplated for use in the methods and compositions herein include rituximab (Rituxan®, Genentech), ofatumumab (Arzerra®, Genmab AC), ocrelizumab (Genentech), veltuzumab (Immunomedics), AME-133V (Eli Lilly), PRO131921 (Genentech), GA101 (Glycart/Roche), ibritumomab tiuxetan (Zevalin), tositumomab (Bexxar), and obinutuzumab (Gazyva®, Genentech). In embodiments, the combination of a long-acting IL-15 receptor agonist such as described herein and an anti-CD20 antibody are used in the treatment of B cell malignancies including, without limitation, non-Hodgkin lymphoma, CLL, diffuse large B cell lymphoma (DLBCL), and follicular lymphoma.

CD38 is a 45 kDa type II transmembrane glycoprotein having receptor as well as enzyme functions. CD38 is generally expressed at low levels on various hematological and solid tissues, but expressed at high levels by plasma cells (shows especially broad and high expression levels in plasma cell tumors such as multiple myeloma (MM)). CD38 is also expressed in a subset of hematological tumors. In some further embodiments, the instant combination comprises administration of a long-acting IL-15 receptor agonist and an anti-CD38 monoclonal antibody. Some exemplary anti-CD38 antibodies contemplated for use in the methods and compositions provided herein include daratumumab (DARZALEX®, Janssen Biotech), isatuximab (SAR650984, Sanofi Oncology), and MOR202 (Morphosys). In some further embodiments, the combination of a long-acting IL-15 receptor agonist and an anti-CD38 antibody are used in the treatment of a condition selected from multiple myeloma, CD38+ non-Hodgkin lymphoma, CDCLL, Waldenstrom's macroglobulinemia, primary systemic amyloidosis, mantle cell lymphoma, acute lymphoblastic leukemia, acute myeloid leukemia, NK cell leukemia, NK/T-cell lymphoma, and plasma cell leukemia.

In some additional embodiments, a therapeutic immuno-oncology based combination includes a long-acting IL-15 receptor agonist as described herein and an antibody directed to a glycoprotein selected from, without limitation, SLAMF7, EpCAM, gpA3, or folate binding protein (FBP). In embodiments, the antibody is an anti-SLAMF7 antibody, an anti-EpCAM antibody, an anti-gpA3 antibody, or an anti-FBP antibody.

The Signaling Lymphocyte Activation Molecule Family Member 7 (SLAMF7, previously known as CS1, CD319, CRACC) is a member of the signaling lymphocytic activation molecule family. SLAMF7 is expressed on immune cells, such as B cells, T cells, dendritic cells, NK T cells, and monocytes, as well as on multiple myeloma cells. In embodiments of the instant combination, the antibody is an anti-SLAMF7 monoclonal antibody. One exemplary anti-SLAMF7 antibody contemplated for use in the methods and compositions herein is elotuzumab (Emplicity™, Bristol-Myers Squibb). In embodiments, the combination of long-acting IL-15 receptor agonist and anti-SLAMF7 antibody are used in the treatment of multiple myeloma.

Epithelial cell adhesion molecule (EpCAM) is a transmembrane glycoprotein that is expressed by a number of epithelial cancer cells including tumors of gastrointestinal origin and some cancers of the genitourinary trace. EpCAM is expressed in, for example, human colon carcinoma, metastatic breast cancer, gallbladder cancer, ovarian cancer, and pancreatic cancer. In embodiments of the therapeutic combination provided herein, the antibody is an anti-EpCAM monoclonal antibody. Some exemplary anti-EpCAM antibodies contemplated for use in the methods and compositions herein include edrecolomab (Panorex, Creative Biolabs), ING-1 (Xoma), 3622W94 (Creative Biolabs), and adecatumumab (Amgen). In one or more embodiments, the combination of a long-acting IL-15 receptor agonist and an anti-EpCAM antibody are used in the treatment of human colon carcinoma, metastatic breast cancer, gallbladder cancer, ovarian cancer, adenocarcinomas, and pancreatic cancer.

In some further embodiments, the antibody is directed to a growth factor selected from, without limitation, vascular endothelial growth factor (VEGF), vascular endothelial growth factor receptor (VEGFR), and epidermal growth factor receptor (EGFR) for example. In some embodiments, the antibody is selected from an anti-VEGF antibody, an anti-VEGFR antibody, and an anti-EGFR antibody.

Vascular endothelial growth factor (VEGF) is a 27 kDa angiogenic signaling protein. VEGF is expressed in a most types of non-digestive and digestive cancers including pancreatic cancer, breast cancer, ovarian cancer, colorectal cancer, and lung cancer. Some exemplary anti-VEGF antibodies contemplated for use in the methods and compositions herein include bevacizumab (Avasatin®, Genentech, Inc.), ranibizumab, 2C3 and r84 (AT001, Affitech AS), and VEGF-Trap (aflibercept, Regeneron Pharmaceuticals, Inc.). In one or more embodiments, the combination of a long-acting IL-15 receptor agonist and an anti-VEGF antibody is used in the treatment of a cancer such as, for example, pancreatic cancer, breast cancer, ovarian cancer, colorectal cancer, metastatic renal cell carcinoma, and non-small cell lung cancer.

Vascular endothelial growth factor receptor (VEGFR) is a receptor tyrosine kinase (RTK) that can induce angiogenesis, increase cell growth and metastasis, etc. The VEGFR family has three main subtypes: VEGFR-1, VEGFR-2 and VEGFR-3. Some exemplary anti-VEGFR antibodies contemplated for use in the methods and compositions herein include MF1/IMC-18F1 (ImClone Systems); IMC-1121B ImClone Systems), and DC101/IMC-1C11. In some further embodiments, the combination of a long-acting IL-15 receptor agonist and an anti-VEGFR antibody is used in the treatment of a cancer such as, for example, breast cancer or non-small cell lung cancer. In some embodiments, the anti-VEGFR antibody is used for an indication as recited above for an anti-VEGF antibody.

Epidermal growth factor receptors (EGFR) are a large family of receptor tyrosine kinases expressed in several types of cancer including breast, lung, esophageal, metastatic colorectal, and head and neck. Some exemplary anti-EGFR antibodies contemplated for use in the methods and compositions herein include cetuximab (Erbitux®, Lilly USA) and panitumumab (Vectibix®, Amgen). In one or more particular embodiments, the antibody is an anti-human epidermal growth factor receptor 2 antibody (anti-HER2). Some exemplary anti-HER2 antibodies contemplated for use in the methods and compositions herein include the humanized monoclonal antibody trastuzumab (Herceptin®, Genentech, Inc.) and/or pertuzumab (Perjeta®, Genentech, Inc.). In some related embodiments, the combination of a long-acting IL-15 receptor agonist and an anti-EGFR antibody is used in the treatment of a cancer such as breast (e.g. metastatic breast cancer), lung, esophageal, metastatic colorectal, and head and neck cancers.

Also contemplated for use in the methods and combinations provided herein is an anti-BCMA (B-cell maturation antigen, also referred to as CD269) antibody. BCMA is a member of the tumor necrosis factor receptor (TNFR) superfamily. BCMA binds B-cell activating factor (BAFF) and a proliferation inducing ligand (APRIL). Among non-malignant cells, BCMA has been reported to be expressed mostly in plasma cells and subsets of mature B-cells. BCMA RNA has been detected in multiple myeloma cells, and BCMA protein has been detected on the surface of plasma cells from multiple myeloma patients. BCMA is expressed or overexpressed by various human cancers. Examples of cancers that express or overexpress BCMA include, but are not limited to, Burkitt's lymphoma, diffuse large B-cell lymphoma (DLBCL) lymphoma, acute lymphocytic leukemia (ALL) lymphoma, Hodgkin's lymphoma and multiple myeloma. Exemplary anti-BCMA antibodies are described, for example, in U.S. Patent Publication Nos. 20120082661, 20170051068, and 20180318435 (the contents each of which is incorporated herein by reference in its entirety). Exemplary anti-BCMA antibodies include BCMAxXD3, described in Pillarisetti, K., et al., Blood, 2016, 128:2116, and the humanized antibody portion of the anti-BCMA antibody drug conjugate, GSK2857916.

In some embodiments, the antibody may simultaneously bind more than one specific antigen, e.g. a bispecific antibody.

In some embodiments, the antibody is one that cross-competes for binding with any of the antibodies as described above. An antibody that cross-competes with any one of the above-referenced antibodies is expected to have similar or the same functional properties.

In some embodiments, the antibodies for use herein are the antigen-binding portion of any of the antibodies as described above as it is well known in the art that the antigen-binding function of an antibody may be performed by fragments of the full-length antibody.

Turning now to the long-acting IL-15 receptor (IL-15R) agonist, generally, a preferred long-acting IL-15 receptor agonist or a pharmaceutically acceptable salt form thereof comprises a single linear polyalkylene oxide (e.g. polyethylene glycol or “PEG”) moiety stably covalently attached to an IL-15 amino group via an amide linkage. Intervening between the PEG moiety and the stable amide linkage to an IL-15 amino group is a linear unsubstituted alkylene group (˜CH₂˜)ₘ having from 2 to 5 carbon atoms (i.e., m is 2, 3, 4, or 5). In one or more preferred embodiments, m is 3 such that the stable amide linkage is a butanamide.

In one or more preferred embodiments, the long-acting IL-15R agonist is (methoxyPEG-N-butanamide)_{20-60 kD} interleukin-15, more preferably (methoxyPEG-N-butanamide)_{20-40 kD} interleukin-15, and even more preferably (methoxyPEG-N-butanamide)_{40 kD} interleukin-15. In one or more preferred embodiments, the long-acting IL-15R agonist is mono(methoxyPEG-N-butanamide)_{20-60 kD} interleukin-15, more preferably mono(methoxyPEG-N-butanamide)_{20-40 kD} interleukin-15, and even more preferably mono(methoxyPEG-N-butanamide)_{40 kD} interleukin-15. In one preferred embodiment, the long-acting IL-15R agonist is mono(methoxyPEG-N-butanamide)_{40 kD} interleukin-15.

IL-15R agonists described herein having a structure encompassed by Formula (I), and in particular mono(methoxyPEG-N-butanamide)_{40 kD} interleukin-15, retain binding affinity to the IL-15 receptor alpha (IL-15Rα) subunit as well as the beta (β) and gamma (γ) subunits. The long-acting IL-15R agonists described herein present to the IL-2/IL-15βγ complex on the same (cis) or adjacent cells (trans). Engagement of the IL-2/IL-15βγ complex by the long-acting IL-15R agonists described herein can induce the Janus kinase/signal transducer and activator of transcription-5 (JAK-STAT5) pathways, which increases T cell proliferation, survival and/or activity. The long-acting IL-15R agonist further significantly enhances proliferation and activation of CD8+ T cells. The long-acting IL-15R agonists preferably have decreased clearance as compared to corresponding unmodified IL-15R agonists. Without being limited by theory, it is postulated that attachment of the PEG enlarges the hydrodynamic volume of the IL-15 moiety to result in a longer effective half-life, lower maximum peak concentration (Cₘₐₓ), and/or reduced clearance in comparison to unconjugated rhIL-15. The long-acting IL-15R agonists described herein provide sustained IL-15 biological activity without the need for daily dosing.

When considering the IL-15 moiety, the term “IL-15 moiety” refers to the IL-15 moiety prior to conjugation as well as to the IL-15 moiety following attachment to a non-peptidic, water-soluble polymer such as PEG. While specific reference is made to PEG hereafter as the non-peptidic, water-soluble polymer below, it will be understood that the disclosure relates generally to a non-peptidic, water-soluble polymer or poly(alkylene glycol). It will be understood, however, that when the original IL-15 moiety is attached to a polyethylene glycol moiety, the IL-15 moiety is slightly altered due to the presence of one or more covalent bonds associated with linkage to the polymer(s).

The IL-15 moiety can be derived from non-recombinant methods and from recombinant methods, and the disclosure is not limited in this regard. In addition, the IL-15 moiety can be derived from human sources, animal sources (including insects), fungi sources (including yeasts), and plant sources.

The IL-15 moiety can be obtained according to the procedures described by, for example, Grabstein et al. (Grabstein et al. (1994) Science 264:965-968). The IL-15 moiety can also be prepared using recombinant methods, such as, for example, those described in EP Patent No. 0 772 624 B2 to Immunex Corporation. Alternatively, the IL-15 moiety can be purchased commercially from, for example, GenScript USA Inc. (Piscataway N.J.) and Peprotech (Rockyhill, N.J.).

The IL-15 moiety can be expressed in bacterial (e.g., E. coli, see, for example, Fischer et al. (1995) Biotechnol. Appl. Biotechnol. 21(3):295-311), mammalian (see, for example, Kronman et al. (1992) Gene 121:295-304), yeast (e.g., Pichia pastoris, see, for example, Morel et al. (1997) Biochem. J. 328(1):121-129), and plant (see, for example, Mor et al. (2001) Biotechnol. Bioeng. 75(3):259-266) expression systems. The expression can occur via exogenous expression (when the host cell naturally contains the desired genetic coding) or via endogenous expression.

Further methods of preparation and/or purification of an IL-15 moiety are described in PCT Application No. PCT/US2018/032817, which is incorporated by reference herein in its entirety.

Depending on the system used to express proteins having IL-15 activity, the IL-15 moiety can be unglycosylated or glycosylated and either may be used. In one or more embodiments, the IL-15 moiety is unglycosylated.

The IL-15 moiety can advantageously be modified to include and/or substitute one or more amino acid residues such as, for example, lysine, cysteine and/or arginine, in order to provide facile attachment of the polymer to an atom within the side chain of the amino acid. An example of substitution of an IL-15 moiety is described in U.S. Pat. No. 6,177,079. In addition, the IL-15 moiety can be modified to include a non-naturally occurring amino acid residue. Techniques for adding amino acid residues and non-naturally occurring amino acid residues are well known to those of ordinary skill in the art. Reference is made to J. March, Advanced Organic Chemistry: Reactions Mechanisms and Structure, 4th Ed. (New York: Wiley-Interscience, 1992), and Bioinformatics for Geneticists (eds. Michael R. Barnes and Ian C Gray), 2003 John Wiley & Sons, Ltd, Chapter 14, Amino Acid Properties and Consequences of Substitutions, Betts, M. J., and Russell, R. B.

Additional suitable modifications and methods for such modification of the IL-15 moiety are described in PCT Application No. PCT/US2018/032817, which is incorporated by reference herein in its entirety. Exemplary modifications include attachment of a functional group (other than through addition of a functional group-containing amino acid residue), such as, for example, inclusion of a thiol group, an N-terminal alpha carbon, one or more carbohydrate moieties, an aldehyde group, or a ketone group. In some embodiments, it is preferred that the IL-15 moiety is not modified to include one or more of a thiol group, an N-terminal alpha carbon, a carbohydrate, an aldehyde group, or a ketone group.

Exemplary IL-15 moieties are described herein, in the literature, and in, for example, U.S. Patent Application Publication No. US 2006/0104945, Pettit et al. (1997) J. Biol. Chem. 272(4):2312-2318, Wong et al. (2013) OncoImmunology 2(11), e26442:1-3, and PCT Application No. PCT/US2018/032817, each of which is incorporated by reference herein in its entirety. Preferred IL-15 moieties include those having an amino acid sequence comprising sequences selected from the group consisting of SEQ ID NOs: 1 through 3, and sequences substantially homologous thereto (wherein even if SEQ ID NOs 2 and 3, and sequences substantially homologous thereto do not meet the in vitro activity standard of an IL-15 moiety provided herein, it will be understood for purposes of the present disclosure that these sequences are also understood to be “IL-15 moieties”). A preferred IL-15 moiety has an amino acid sequence corresponding to SEQ ID NO: 1. In some embodiments, the IL-15 moiety is a functional homolog having at least about 85% or at least about 90% identity with any one of SEQ ID NOs: 1-3. In some embodiments, the IL-15 moiety is a functional homolog having at least about 95%, 98% or 99% identity with any one of SEQ ID NOs: 1-3.

In some instances, the IL-15 moiety will be in a “monomer” form, wherein a single expression of the corresponding peptide is organized into a discrete unit. In other instances, the IL-15 moiety will be in the form of a “dimer” (e.g., a dimer of recombinant IL-15) wherein two monomer forms of the protein are associated to each other.

In addition, precursor forms of IL-15 can be used as the IL-15 moiety. An exemplary precursor form of IL-15 has the sequence of SEQ ID NO: 3.

In some embodiments, the long-acting IL-15R agonist is a PEGylated IL-15 molecule as described in U.S. Published Application No. 2018/0360977, and specifically a multi-arm PEG IL-15 as described therein.

In some embodiments, the IL-15 moiety is an IL-15 mutein or other IL-15 related molecule as described in U.S. Pat. No. 10,350,270.

Truncated versions, hybrid variants, and peptide mimetics of any of the foregoing sequences can also serve as the IL-15 moiety. Biologically active fragments, deletion variants, substitution variants or addition variants of any of the foregoing that maintain at least some degree of IL-15 activity can also serve as an IL-15 moiety.

For any given peptide, protein moiety or conjugate, it is possible to determine whether that peptide, protein moiety or conjugate has IL-15 activity. Various methods for determining in vitro IL-15 activity are described in the art. An exemplary approach is based on a pSTAT assay. Briefly, if an IL-15-dependent CTLL-2 cell is exposed to a test article having IL-15 activity, initiation of a signaling cascade results that includes the phosphorylation of STAT5 at tyrosine residue 694 (Tyr694), which can be quantitatively measured. Assay protocols and kits are known and include, for example, the MSD Phospho(Tyr694)/Total STATa,b Whole Cell Lysate Kit (Meso Scal Diagnostics, LLC, Gaithersburg, Md.). For example, using this approach, a proposed IL-15 moiety that exhibits a pSTAT5 EC₅₀ value of no more than about 300 ng/mL (more preferably no more than about 150 ng/mL) at least one of 5 minutes or at 10 minutes is considered an “IL-15 moiety” in connection with the present disclosure. It is preferred, however, that the IL-15 moiety used is more potent (e.g., having a pSTAT5 EC₅₀ value of less than 150 ng/mL at one of least 5 minutes or 10 minutes, such as less than about 1 ng/mL, and even more preferably less than 0.5 ng/mL at least one of 5 minutes or at 10 minutes).

Other methodologies known in the art can also be used to assess IL-15 function, including electrometry, spectrophotometry, chromatography, and radiometric methodologies. See, for example, Ring et al. (2012) Nat. Immunol. 13(12):1187-1195 for one such additional type of assay.

Assays for use in connection with measuring the activity of an IL-15 moiety can also be used to measure the activity of the long-acting IL-15 receptor agonists described herein. See, for example, the supporting examples provided herein.

A compound is considered to be a long-acting, IL-15 receptor agonist in accordance with the present disclosure so long as, following administration to a subject, the agonist exhibits IL-15R agonism in vivo for an amount of time that is longer than would be the case for administration of IL-15. Conventional approaches, such as those involving radiolabeling a compound, administering the compound in vivo, and determining its clearance, can be used to assess whether a compound proposed to be a long-acting, IL-15 receptor agonist is “long-acting” (i.e., has a clearance that is longer than that of IL-15 administered in the same in vivo system). For the purposes herein, the long-acting nature of a long-acting IL-15 receptor agonist may be, and is typically determined using flow cytometry to measure STAT5 phosphorylation in lymphocytes at various time points after administration of the agonist to be evaluated in mice. As a reference, the signal is lost by around 24 hours with IL-15, but is sustained for a period greater than that for a long-acting IL-15 agonist.

A preferred long-acting IL-15 receptor agonist will generally comprise a single linear PEG (polyethylene glycol) moiety stably covalently attached to an IL-15 amino group via an amide linkage. Intervening between the PEG moiety and the stable amide linkage to an IL-15 amino group is a linear unsubstituted alkylene group (˜CH₂˜)ₘ having from 2 to 5 carbon atoms (i.e., where m=2, 3, 4, or 5).

For example, in some embodiments, the unsubstituted alkylene group is (˜CH₂˜)₂; or, in some additional embodiments, the unsubstituted alkylene group is (˜CH₂˜)₃; in yet some further embodiments, the unsubstituted alkylene group is (˜CH₂˜)₄; in yet some further embodiments, the unsubstituted alkylene group is the unsubstituted alkylene group is (˜CH₂˜)₅.

For example, in some embodiments, the long-acting IL-15 receptor agonist has the following structure:

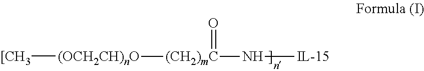

wherein IL-15 is an interleukin-15 moiety, n is an integer from about 150 to about 3,000; m is an integer from 2-5 (e.g., 2, 3, 4, or 5) and n′ is 1. In Formula I (and in similar formulae provided herein) the ˜NH˜ in the structure represents an amino group of the IL-15 moiety. Formula (I) may also be depicted as follows, where the parentheses are shifted to reflect a terminal PEG methoxy group,

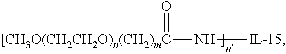

and the two formulae may be used interchangeably. Illustrative exemplary compounds include the following encompassed b Formula (I):

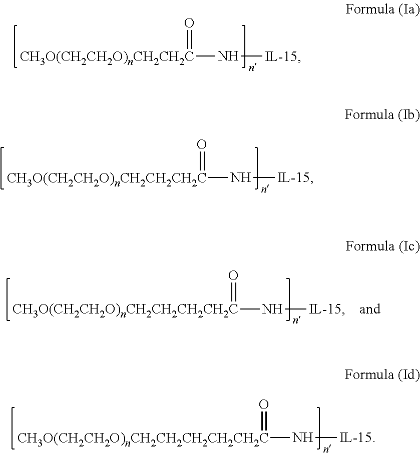

In some preferred embodiments, the long-acting IL-15 receptor agonist corresponds to Formula (Ia) or Formula (Ib). In some particularly preferred embodiments, the long-acting IL-15 receptor agonist corresponds to Formula (Ib).

In some further embodiments, in reference to the structures and formulae described herein, n is an integer from about 200 to about 2000, or from about 400 to about 1300, or from about 450 to about 1200. That is to say, in some embodiments, n is an integer from about 200 to about 2000. In yet some further embodiments, n is an integer from about 400 to about 1300. In yet some further embodiments, n is an integer from about 450 to about 1200.

PEGs having a molecular weight corresponding to any one of the foregoing ranges of n values are generally preferred.

In one or more additional embodiments, n is an integer having a value that corresponds to a polyethylene glycol polymer having a weight average molecular weight selected from the group consisting of about 10,000 daltons (where n is ˜227), or about 15,000 daltons (where n is ˜340), or about 20,000 daltons (where n is ˜454), or about 25,000 daltons (where n is ˜568), or about 30,000 daltons (where n is ˜681), or about 40,000 daltons (where n is ˜907-909, e.g., ˜909), or about 50,000 daltons (where n is ˜1136) or even about 60,000 daltons (where n is ˜1364) or greater.

Further exemplary weight-average molecular weights for the polyethylene glycol portion of the compound, in addition to the foregoing, include about 11,000 daltons, about 12,000 daltons, about 13,000 daltons, about 14,000 daltons, about 22,500 daltons, about 35,000 daltons, about 40,000 daltons, about 45,000 daltons, about 55,000 daltons, about 65,000 daltons, about 70,000 daltons, and about 75,000 daltons. In some embodiments, the weight-average molecular weight of the polyethylene glycol portion of the IL-15 conjugate is about 37,000 to about 45,000 daltons.

In some preferred embodiments, the weight-average molecular weight of the polyethylene glycol polymer portion of the IL-15 conjugate is about 40,000 daltons. In reference to each of Formulae (I) and (II) described herein, including any sub-formulae thereto, a weight average molecular weight of PEG of 40,000 daltons is preferred.

While the PEG moiety is preferably end-capped as shown above in Formula (I) with a methoxy group, the PEG moiety may be capped at its terminus with any lower C₁₋₆ alkoxy group, or may terminate in a hydroxyl group, or other suitable end-capping group.

In some embodiments, the long-acting IL-15 receptor agonist comprises no more than about 20 mole percent (mol %) of long-acting IL-15 receptor agonists (of the IL-15 containing molecules in the composition), when considered collectively, encompassed by the formula:

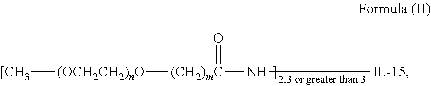

where the values of n and m are as provided for Formula (I) above. That is to say, a monoPEGylated long-acting IL-15 receptor agonist, e.g., of any of Formulae (I), (Ia), (Ib), (Ic), and (Id), e.g., of Formula (Ib), for example, when comprised in a composition of long-acting IL-15 receptor agonist compounds, e.g., of the same chemical formula but having different numbers of PEG moieties covalently attached to the interleukin-15 moiety (e.g., 2-mers, 3-mers, etc.), comprises no more than about 20 mole percent of long-acting IL-15 receptor agonists of Formula (II).

In some additional embodiments, the long-acting IL-15 receptor agonist composition comprises no more than about 15 mole percent (mol %) of long-acting IL-15 receptor agonists (of the IL-15 containing molecules in the composition), when considered collectively, encompassed by the formula:

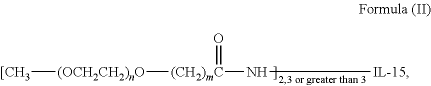

where the values of n and m are as provided for Formula (I) above. That is to say, in terms of the long-acting IL-15 receptor agonist component of such compositions, no more than about 15 mol % of long-acting IL-15 receptor agonists comprised in the composition are of Formula (II). In some embodiments, the long-acting IL-15 receptor agonist composition comprises no more than about 0.1-20 mol % of compounds of Formula (II). In embodiments, the compositions comprise no more than about 0.1-15, 0.1-10, 0.1-5, 0.1-1, 1-20, 1-15, 1-10, 1-5, 5-20, 5-15, 5-10, 10-20, 10-15, or 15-20 mol % of compounds of Formula (II). Preferred embodiments are those in which “m” in Formula (II) is 3.

In some particular embodiments related to the foregoing, the long-acting IL-15 receptor agonist composition, in reference to Formula (Ia), comprises no more than about 15 mole percent (mol %) of long-acting IL-15 receptor agonists (of the IL-15 containing molecules in the composition), when considered collectively, encompassed by the formula:

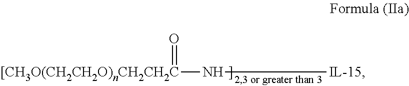

where the values of n and m are as provided for Formula (Ia) above.

In some particularly preferred embodiments, the long-acting IL-15 receptor agonist composition, in reference to Formula (Ib), comprises no more than about 15 mole percent (mol %) of long-acting IL-15 receptor agonists (of the IL-15 containing molecules in the composition), when considered collectively, encompassed by the formula.

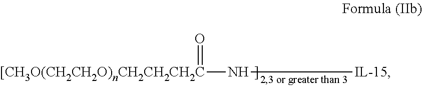

where the values of n and m are as provided for Formula (Ib) above.

In some other embodiments, the long-acting IL-15 receptor agonist composition, in reference to Formula (Ic), comprises no more than about 15 mole percent (mol %) of long-acting IL-15 receptor agonists (of the IL-15 containing molecules in the composition), when considered collectively, encompassed by the formula:

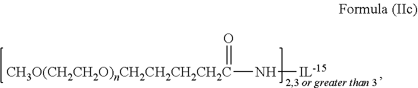

where the values of n and m are as provided for Formula (Ic) above.

In some other embodiments, the long-acting IL-15 receptor agonist composition, in reference to Formula (Id), comprises no more than about 15 mole percent (mol %) of long-acting IL-15 receptor agonists (of the IL-15 containing molecules in the composition), when considered collectively, encompassed by the formula:

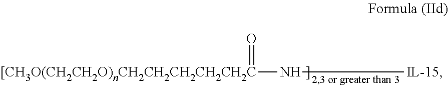

where the values of n and m are as provided for Formula (Id) above.

In some embodiments, the long-acting IL-15 receptor agonist composition comprises no more than about 0.1-20 mol % of compounds of Formula (II), including compounds of Formulae (IIa), (IIb), (IIc), and (IId). In some additional embodiments, the compositions comprise no more than about 0.1-15, 0.1-10, 0.1-5, 0.1-1, 1-20, 1-15, 1-10, 1-5, 5-20, 5-15, 5-10, 10-20, 10-15, or 15-20 mol % of compounds of Formula (II), including compounds of Formulae (IIa), (IIb), (IIc), and (IId). In some embodiments, the compositions comprise no more than about 0.1, 1, 5, 10, 15, or 20 mol % of compounds of Formula (II), including compounds of Formulae (IIa), (IIb), (IIc), and (IId). It will be appreciated that the compositions may be purified by methods known in the art for compounds of Formula (I) resulting in no, trace amounts, or substantially no compounds of Formula (II) present in the composition.

For example, in some embodiments, the long-acting IL-15 receptor agonist composition comprises no more than about 12 mole percent, or no more than about 10 mole percent of long-acting IL-15 receptor agonists, that when considered collectively, are encompassed by Formula (II), including compounds of Formulae (IIa), (IIb), (IIc), and (IId).

In some additional embodiments of the foregoing, the composition comprises no more than about 7 mol % of long-acting IL-15 receptor agonists having n′ equal to 2, 3, or greater than 3 (i.e., higher PEGmers). In yet some other embodiments, the composition comprises no more than about 5 mol % of long-acting IL-15 receptor agonists having n′ equal to 2, 3 or greater than 3 (i.e., of 2 or greater).

In some further embodiments, the composition comprises a long-acting IL-15 receptor agonist according to Formula (I),

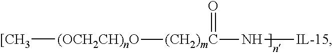

where n and m are as described above, and n′ represents the average number of polyethylene glycol moieties covalently attached to IL-15 amino groups (for the composition), and n′ for the composition is in a range from 1.0 to about 1.3. For example, the average number of polyethylene glycol moieties per IL-15 moiety is selected from about 1.0, 1.1, 1.2 and about 1.3. That is to say, a preferred long-acting IL-15 receptor agonist according to Formula (I) may be referred to herein as “monoPEGylated”, where it is to be understood that some variability exists around the degree of PEGylation as described above. In some preferred embodiments in reference to the formulae described herein, “m” is equal to 3.

A composition of the long-acting IL-15 receptor agonist may comprise a single species where n′ equals about 1 and the PEG moiety is attached at the same location for substantially all IL-15 conjugates in the composition, or alternatively, may comprise a mixture of monoPEGylated conjugate species where attachment of the linear polyethylene glycol moiety occurs at different sites on the interleukin-15 moiety, that is to say, where the particular attachment sites are not the same for all of the monoPEGylated IL-15 species comprised in the composition). Thus, such compositions are substantially homogeneous in terms of the number of PEG moieties attached to IL-15 (e.g., 1-mers), but are heterogeneous in terms of the locations of amino group attachment on the IL-15 molecule.

While additional PEG architectures and linkage chemistries may be employed for arriving at a long-acting IL-15 receptor agonist, compounds such as previously described are preferred in one or more embodiments, as will become apparent when considered in light of the supporting examples. However, additional long-acting IL-15 receptor agonists having structures as provided herein are also contemplated.

In some embodiments, the long-acting IL-15 receptor agonist composition comprises at least about 80 mol % of long-acting IL-15 receptor agonists (of the IL-15 containing molecules in the composition), when considered collectively, encompassed by Formula (I), including Formulae (Ia-d). In one or more embodiments, the long-acting IL-15 receptor agonist composition comprises at least about 85 mol %, 90 mol %, 95 mol %, 98 mol % or 99 mol % of the long-acting IL-15 receptor agonists of Formula (I), where a particularly preferred formula is that of Formula (Ib).

Every compound as described herein, including the long-acting IL-15 receptor agonist, is meant to expressly include such compounds in the form of a pharmaceutically-acceptable salt. Typically, such salts (e.g., of a basic compound) are formed by reaction with a pharmaceutically-acceptable acid or an acid equivalent. The term “pharmaceutically-acceptable salt” in this respect, will generally refer to a relatively non-toxic, inorganic or organic acid addition salt. These salts can be prepared in situ in the administration vehicle or the dosage form manufacturing process, or by separately reacting a long-acting interleukin-15 receptor agonist as described herein with a suitable organic or inorganic acid, and isolating the salt thus formed. Representative salts include, but are not limited to, the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, oxylate, mesylate, glucoheptonate, lactobionate, and laurylsulphonate salts and the like. (See, for example, Berge et al. (1977) “Pharmaceutical Salts”, J. Pharm. Sci. 66:1-19). Thus, salts as described may be derived from inorganic acids such as hydrochloride, hydrobromic, sulfuric, sulfamic, phosphoric, nitric, and the like; or prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In some embodiments, the long-acting IL-15 receptor agonist composition comprises no more than about 1-5 mol % of free IL-15 protein (of the IL-15 containing molecules in the composition), when considered collectively. In some further embodiments, the long-acting IL-15 agonist composition comprises no more than about 0.5 mol %, 1 mol %, 2 mol %, 3 mol %, 4 mol %, or 5 mol % of free (i.e., unconjugated) IL-15.

To prepare a long-acting IL-15 receptor agonist, the IL-15 moiety may, for example, be conjugated at its amino groups (e.g. lysines or the N-terminus) to a PEG reagent functionalized with a succinimidyl group (or other activated ester group). Using this approach, the succinimidyl activated PEG can be attached to amino groups on the IL-15 moiety in an aqueous media at a pH of about 7.0 to 9.0, although using different reaction conditions (e.g., a lower pH such as 6 to 7 or 7 to 8, or different temperatures and/or less than 15° C.) can result in the attachment of the PEG moiety to a different location on the IL-15 moiety.

Exemplary methods for preparing a long-acting IL-15 receptor agonist are described in PCT Application No. PCT/US2018/032817, e.g. Examples 1-2, which is incorporated by reference herein in its entirety. In one exemplary embodiment, the long-acting IL-15 receptor agonist may generally be prepared by reacting an interleukin-15, for example, purified IL-15, such as recombinant IL-15, with an activated PEG reagent such as the activated ester, methoxyPEG-succinimidyl butanoate, mPEG-SBA. Other suitable activated PEG reagents include methoxyPEG-succinimidyl propionate, methoxyPEG-succinimidyl pentanoate, and methoxyPEG-succinimidyl hexanoate. While a succinimidyl activating group is typically used, any suitable active ester or activating group may be used, wherein such reacting group is suitable for forming the desired stable amide linkage. The PEGylated IL-15 reaction product may then generally be purified by any suitable method such as, for example, ion exchange chromatography to obtain the desired product. For example, anion exchange chromatography may be employed. The chromatography product pool may then be concentrated and diafiltered into suitable formulation buffer (for example, sodium acetate buffer with sucrose) using, for example, tangential flow filtration (TFF). Analysis may be conducted by any suitable method, such as for example, SDS-PAGE, reverse phase HPLC or any other suitable analytical method.

As described previously, amino groups on the IL-15 moiety provide a site of attachment between the IL-15 moiety and the polyethylene glycol moiety to provide long-acting IL-15 receptor agonists such as encompassed by Formula (I). Considering the exemplary IL-15 amino acid sequences provided herein, it is evident that there are seven lysine residues each having an ε-amino acid that may be available for conjugation for at least SEQ ID NO:1 and 2. Further, the N-terminal amine of methionine can also serve as a point of attachment to the PEG moiety. It will be appreciated that the polyethylene glycol moiety may be attached at any one or more of the lysine or the N-terminal amine positions. In some embodiments, a polyethylene glycol moiety attachment site is at one or more of Lys¹⁰ and Lys¹¹ (using the numbering as shown in SEQ ID NO: 2 as an example or Lys¹¹ and Lys¹² using SEQ ID NO:1). In some embodiments, a polyethylene glycol moiety is attached at the N-terminal amine. It will be appreciated that any of the lysine sites may be suitable as an attachment site (e.g. Lys³⁷ or Lys⁴² of SEQ ID NO:1) for the PEG moiety. In some embodiments, the long-acting interleukin-15 receptor agonist comprises a mixture of positional isomers, where covalent attachment of the polyethylene glycol moiety is predominately at the N-terminus (that is to say, of the collection of positional isomers, the isomer having the PEG moiety attached at the N-terminus is present in the highest amount, when compared to the other positional isomers).

If desired, the product pool may be further separated into positional isomers by reverse phase chromatography using a reverse phase-high performance liquid chromatography (RP-HPLC) using a suitable column (e.g., a C18 column or C3 column, available commercially from companies such as Amersham Biosciences or Vydac) or by ion exchange chromatography using an ion exchange column, e.g., a Sepharose™ ion exchange column available from Amersham Biosciences. Either approach can be used to separate PEG-interleukin-15 positional isomers having the same molecular weight (i.e., positional isoforms).

The instant long-acting IL-15 receptor agonists have been discovered to possess certain notable and advantageous features. While the features described below are believed to apply generally to compounds as provided herein and encompassed by Formula (I), the following one or more features may be exhibited particularly by compounds in accordance with Formula (Ib), and by extension, Formula (IIb). The long-acting IL-15 receptor agonist may possess one or more of the following features. For example, in some embodiments, the long-acting IL-15 receptor agonist exhibits no more than about a 7-fold reduction in EC50 value (ng/mL, CTLL-2 pSTAT5) when compared to unmodified IL-15. For example, in one or more related embodiments, the long-acting IL-15 receptor agonist exhibits no more than about a 6.5-fold reduction in EC50 value (ng/mL, CTLL-2 pSTAT5), or no more than about a 6-fold reduction in EC50 value (ng/mL, CTLL-2 pSTAT5), or no more than about a 5.5-fold reduction in EC50 value (ng/mL, CTLL-2 pSTAT5), or no more than about a 5-fold reduction in EC50 value (ng/mL, CTLL-2 pSTAT5), or no more than about a 4.5-fold reduction in EC50 value (ng/mL, CTLL-2 pSTAT5), or no more than about a 4-fold reduction in EC50 value (ng/mL, CTLL-2 pSTAT5), or no more than about a 3.5-fold reduction in EC50 value (ng/mL, CTLL-2 pSTAT5), or even no more than about a 3-fold reduction in EC50 value (ng/mL, CTLL-2 pSTAT5) when compared to IL-15. Exemplary long-acting IL-15 receptor agonists in accordance with the foregoing features are described herein and in PCT Application No. PCT/US2018/032817, which is incorporated by reference herein in its entirety.

The amount of the long-acting IL-15 receptor agonist comprised in the composition will vary depending on a number of factors, but will optimally be a therapeutically effective dose when the composition is stored in a unit dose container (e.g., a vial). In addition, a pharmaceutical preparation can be housed in a syringe. A therapeutically effective dose can be determined experimentally by repeated administration of increasing amounts of the long-acting IL-15 receptor agonist in order to determine an amount that produces a clinically desired endpoint as described herein. The amount of any individual excipient in the composition will vary depending on the activity of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects.

Each pharmacological component of the method can be administered separately. Alternatively, if administration of two pharmacological components is desired to be simultaneous—and the two pharmacological components are compatible together and in a given formulation—then the simultaneous administration can be achieved via administration of single dosage form/formulation (e.g., intravenous administration of an intravenous formulation that contains both pharmacologically active agents). One of ordinary skill in the art can determine through routing testing whether two given pharmacological components are compatible together and in a given formulation. It will be appreciated that the antibody and the long-acting, IL-15 receptor agonist may be administered in any order. Further, administration of the antibody and the long-acting, IL-15 receptor agonist may be separated by minutes, hours, or days as needed.

With regard to the frequency of administering the long-acting, IL-15 receptor agonist, one of ordinary skill in the art will be able to determine an appropriate frequency. For example, a clinician can decide to administer the long-acting, IL-15 receptor agonist relatively infrequently (e.g., once every two or three weeks) and progressively shorten the period between dosings as tolerated by the patient. With regard to frequency of administering the antibody, the frequency for these agents can be determined in a similar fashion. In addition, as some long-acting IL-15 receptor agonists and antibodies as described herein are either in advanced clinical testing or commercially available, it is also possible to refer to the literature to obtain an appropriate frequency of administration (keeping in mind that some adjustment may be necessary in view of the combined effects of the treatment regimen).

In some treatment regimens, the long-acting IL-15 receptor agonist is administered as a single dose at the commencement of treatment. The antibody is administered either concurrently with the long-acting IL-15 receptor agonist, prior to administration of the long-acting IL-15 receptor agonist, or following administration of the long-acting IL-15 receptor agonist. For example, in some treatment modalities, the antibody is administered within 7-14 days (before or after) of long-acting IL-15 receptor agonist administration (e.g., on any one of days 1, 2, 3, 4, 5, 6, 7, 8, 9, 10, 11, 12, 13, or 14), where day 1 indicates commencement of treatment. Based upon the long-acting nature of the long-acting IL-15 receptor agonist, such compound may be administered relatively infrequently (e.g., once every three weeks, once every two weeks, once every 8-10 days, once every week, once every three days, etc.). Further, the antibody may be administered according to approved schedules (e.g. daily, two or three times weekly, weekly, every two weeks, etc.). In one exemplary schedule, the long-acting IL-15R agonist is administered on day one and every 21 days thereafter. The antibody is administered weekly, every two weeks or monthly starting 7-14 days after administration of the long-acting IL-15R agonist. It will be appreciated that the dosing schedule for one or both of the long-acting IL-15R agonist and the antibody may be adjusted during the treatment period. As an example, the long-acting IL-15R agonist and/or the antibody may be administered weekly for a period of time (e.g. 4-8 weeks), and then every two weeks or monthly for a period of time thereafter.

The treatment method described herein can continue for as long as the clinician overseeing the patient's care deems the treatment method is effective. Non-limiting parameters that indicate the treatment method is effective include the following: tumor shrinkage (in terms of weight and/or volume); a decrease in the number of individual tumor colonies; tumor elimination; and progression-free survival.

Exemplary lengths of time associated with the course of therapy as described herein include: about one week; two weeks; about three weeks; about four weeks; about five weeks; about six weeks; about seven weeks; about eight weeks; about nine weeks; about ten weeks; about eleven weeks; about twelve weeks; about thirteen weeks; about fourteen weeks; about fifteen weeks; about sixteen weeks; about seventeen weeks; about eighteen weeks; about nineteen weeks; about twenty weeks; about twenty-one weeks; about twenty-two weeks; about twenty-three weeks; about twenty four weeks; about seven months; about eight months; about nine months; about ten months; about eleven months; about twelve months; about thirteen months; about fourteen months; about fifteen months; about sixteen months; about seventeen months; about eighteen months; about nineteen months; about twenty months; about twenty one months; about twenty-two months; about twenty-three months; about twenty-four months; about thirty months; about three years; about four years and about five years. It will be appreciated that the dosing frequency for the antibody and the long-acting IL-15 receptor agonist may be the same or different. Further, the dosing frequency or schedule of the antibody and/or the long-acting IL-15 receptor agonist may be designed to achieve sustained occupancy of the receptor/sustained binding to an antigen as appropriate.

Optionally, the antibody and/or the long-acting IL-15 receptor agonist are comprised in a composition that also comprises one or more pharmaceutically acceptable excipients. Exemplary excipients include, without limitation, those selected from the group consisting of carbohydrates, inorganic salts, antimicrobial agents, antioxidants, surfactants, buffers, acids, bases, amino acids, and combinations thereof.

A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, cyclodextrins, and the like.

The excipient can also include an inorganic salt or buffer such as citric acid, sodium chloride, potassium chloride, potassium phosphate, sodium sulfate, potassium nitrate, sodium phosphate monobasic, sodium phosphate dibasic, and combinations thereof.

The composition or compositions can also include an antimicrobial agent for preventing or deterring microbial growth. Nonlimiting examples of antimicrobial agents suitable for one or more embodiments of the present disclosure include benzalkonium chloride, benzethonium chloride, benzyl alcohol, cetylpyridinium chloride, chlorobutanol, phenol, phenylethyl alcohol, phenylmercuric nitrate, thimerosal, and combinations thereof.

An antioxidant can be present in the composition as well. Antioxidants are used to prevent oxidation, thereby preventing the deterioration of the conjugate or other components of the preparation. Suitable antioxidants for use in one or more embodiments of the present disclosure include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant can be present as an excipient. Exemplary surfactants include: polysorbates, such as “Tween 20” and “Tween 80,” and pluronics such as F68 and F88 (both of which are available from BASF, Mount Olive, N.J.); sorbitan esters; lipids, such as phospholipids such as lecithin and other phosphatidylcholines, phosphatidylethanolamines (although preferably not in liposomal form), fatty acids and fatty esters; steroids, such as cholesterol; and IL-15 chelating agents, such as EDTA, zinc and other such suitable cations.

Acids or bases can be present as an excipient in the composition. Non-limiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumarate, and combinations thereof.

One or more amino acids can be present as an excipient in the compositions described herein. Exemplary amino acids in this regard include arginine, lysine and glycine. Additional suitable pharmaceutically acceptable excipients include those described, for example, in the Handbook of Pharmaceutical Excipients, 7^{th} ed., Rowe, R. C., Ed., Pharmaceutical Press, 2012.

In some embodiments, the long-acting IL-15 receptor agonist is comprised in a composition comprising potassium phosphate and trehalose. In some embodiments, the composition is liquid formulation comprising sterile water for injection. In some other embodiments, the composition is a lyophilized powder suitable for reconstruction.

In accord with the method described herein, the long-acting IL-15 receptor agonist and the antibody are administered to a patient in a therapeutically effective amount. One of ordinary skill in the art can determine how much of a given long-acting IL-15 receptor agonist and/or the antibody is sufficient to provide a clinically relevant amount (e.g. agonistic activity at the IL-15 receptor or antibody binding to the desired receptor/antigen, respectively). For example, one of ordinary skill in the art can refer to the literature and/or administer a series of increasing amounts the long-acting IL-15 receptor agonist and determine which amount or amounts provide clinically agonistic activity of IL-15. It will be appreciated that the therapeutic amount of either or both of the long-acting IL-15 receptor agonist and the antibody may be lower than the therapeutically effective amount of either component when administered alone. The synergism of administering a long-acting IL-15 receptor agonist in combination with an antibody as described herein allows for either a lower dose to be administered (for one or both of the components of the combination) or allows for greater efficacy at a dose for either component as determined separately. Generally, a therapeutically effective amount of the antibody will range from about 0.01 to about 100 mg/kg, inclusive. In some particular, but not limiting embodiments, the antibody is administered or included in a composition at a dose of from about 0.01-50 mg/kg, about 0.05-50 mg/kg, about 0.1-50 mg/kg, about 0.5-50 mg/kg, about 1-50 mg/kg, about 5-50 mg/kg, about 10-50 mg/kg, about 15-50 mg/kg, about 20-50 mg/kg, about 30-50 mg/kg, about 40-50 mg/kg, about 0.01-40 mg/kg, about 0.05-40 mg/kg, about 0.1-40 mg/kg, about 0.5-40 mg/kg, about 1-40 mg/kg, about 5-40 mg/kg, about 10-40 mg/kg, about 15-40 mg/kg, about 20-40 mg/kg, about 30-40 mg/kg, about 0.01-30 mg/kg, about 0.05-30 mg/kg, about 0.1-30 mg/kg, about 0.5-30 mg/kg, about 1-30 mg/kg, about 5-30 mg/kg, about 10-30 mg/kg, about 15-30 mg/kg, about 20-30 mg/kg, about 0.05-25 mg/kg, about 0.1-25 mg/kg, about 0.5-25 mg/kg, about 1-25 mg/kg, about 5-25 mg/kg, about 10-25 mg/kg, about 15-25 mg/kg, about 20-25 mg/kg, about 0.01-20 mg/kg, about 0.05-20 mg/kg, about 0.1-20 mg/kg, about 0.5-20 mg/kg, about 1-20 mg/kg, about 5-20 mg/kg, about 10-20 mg/kg, about 15-20 mg/kg, about 0.01-15 mg/kg, about 0.05-15 mg/kg, about 0.1-15 mg/kg, about 0.5-15 mg/kg, about 1-15 mg/kg, about 5-15 mg/kg, about 10-15 mg/kg, about 0.01-10 mg/kg, about 0.05-10 mg/kg, about 0.1-10 mg/kg, about 0.5-10 mg/kg, about 1-10 mg/kg, about 5-10 mg/kg, about 0.1-5 mg/kg, about 0.5-5 mg/kg, about 1-5 mg/kg, about 0.1-1 mg/kg, about 0.5-1 mg/kg, or about 0.1-0.05 mg/kg. In some embodiments, the antibody is administered at a dose of about 0.01 mg/kg, about 0.05 mg/kg, about 0.1 mg/kg, about 0.5 mg/kg, about 1 mg/kg, about 1.5 mg/kg, about 5 mg/kg, about 10 mg/kg, about 15 mg/kg, about 16 mg/kg, about 20 mg/kg, about 25 mg/kg, about 30 mg/kg, about 40 mg/kg or about 50 mg/kg. It will be appreciated that the therapeutically effective amount of the antibody may be any dose as approved by a governmental regulatory agency.

In some embodiments, the antibody is rituximab and the therapeutically effective amount ranges from about 100 mg/m²-750 mg/m² per administration, inclusive. In embodiments, the therapeutically effective amount ranges from about 100 mg/m²-500 mg/m², about 150 mg/m²-500 mg/m², about 200 mg/m²-500 mg/m², about 375 mg/m²-500 mg/m², or about 400 mg/m²-500 mg/m² per administration. In some embodiments, the antibody is administered at a dose of about 100 mg/m², about 150 mg/m², about 200 mg/m², about 250 mg/m², about 375 mg/m², about 400 mg/m², about 500 mg/m², or about 750 mg/m².

In some embodiments, the antibody is daratumumab and the therapeutically effective amount ranges from about 10 mg/kg-50 mg/kg per administration, inclusive. In embodiments, the therapeutically effective amount ranges from about 10-25 mg/kg, about 10-20 mg/kg, about 10-16 mg/kg, about 10-15 mg/kg, about 15-50 mg/kg, about 15-25 mg/kg, about 15-20 mg/kg, about 16-50 mg/kg, about 16-25 mg/kg, or about 16-20 mg/kg per administration. In some embodiments, the antibody is administered at a dose of about 10 mg/kg, about 15 mg/kg, about 16 mg/kg, about 20 mg/kg, about 25 mg/kg or about 50 mg/kg per administration.

In some embodiments, the antibody is cetuximab and the therapeutically effective amount ranges from about 100 mg/m²-750 mg/m² per administration, inclusive. In embodiments, the therapeutically effective amount ranges from about 100 mg/m²-500 mg/m², about 150 mg/m²-500 mg/m², about 200 mg/m²-500 mg/m², about 250 mg/m²-500 mg/m², about 400 mg/m²-500 mg/m², about 200 mg/m²-400 mg/m², about 250 mg/m²-400 mg/m², or about 300 mg/m²-400 mg/m² per administration. In some embodiments, the antibody is administered at a dose of about 100 mg/m², about 150 mg/m², about 200 mg/m², about 250 mg/m², about 400 mg/m², about 500 mg/m², or about 750 mg/m².

In some embodiments, the antibody is trastuzumab and the therapeutically effective amount ranges from about 1 mg/kg-20 mg/kg per administration. In embodiments, the therapeutically effective amount ranges from about 2-20 mg/kg, about 2-15 mg/kg, about 2-10 mg/kg, about 2-8 mg/kg, about 2-6 mg/kg, about 2-4 mg/kg, about 4-10 mg/kg, about 4-8 mg/kg, about 4-6 mg/kg, about 6-10 mg/kg, about 6-8 mg/kg, or about 8-10 mg/kg. In some embodiments, the antibody is administered at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 15 mg/kg or about 20 mg/kg.

It will be appreciated that the dose may be determined by known methods in the art and the specific dose may be set by a regulatory body. A given dose can be periodically administered up until, for example, the clinician determines an appropriate endpoint (e.g., cure, regression, partial regression, and so forth) is achieved.

Generally, a therapeutically effective amount of the long-acting IL-15 receptor agonist will range from about 0.0001 mg to about 100 mg, or about 0.001 mg to about 100 mg, preferably in doses from about 0.01 mg/day to about 75 mg/day, and more preferably in doses from about 0.10 mg/day to about 50 mg/day. A given dose can be periodically administered up until, for example, the clinician determines an appropriate endpoint (e.g., cure, regression, partial regression, and so forth) is achieved.

In some embodiments, the therapeutically effective amount of the long-acting IL-15R agonist ranges from about 0.25-25 μg/kg. In other embodiments, the therapeutically effective dose ranges (e.g. per day) from about 0.25 μg/kg to about 0.1 mg/kg, about 0.375 μg/kg to about 20 μg/kg, about 0.375 μg/kg to about 15 μg/kg, about 0.375 μg/kg to about 10 μg/kg, about 0.375 μg/kg to about 5.0 μg/kg, about 0.375 μg/kg to about 1.5 μg/kg, about 1.0 μg/kg to about 20 μg/kg, about 1.0 μg/kg to about 15 μg/kg, about 1.0 μg/kg to about 10 μg/kg, about 1.0 μg/kg to about 5.0 μg/kg, about 1 μg/kg to about 1.5 μg/kg, about 1.5 μg/kg to about 20 μg/kg, about 1.5 μg/kg to about 15 μg/kg, about 1.5 μg/kg to about 10 μg/kg, about 1.5 μg/kg to about 5.0 μg/kg, about 5.0 μg/kg to about 20 μg/kg, about 5.0 μg/kg to about 15 μg/kg, about 5.0 μg/kg to about 10 μg/kg, about 10 about 1.5 μg/kg to about 20 μg/kg, about 10 μg/kg to about 20 μg/kg, about 10 μg/kg to about 15 μg/kg, about 15 μg/kg to about 20 μg/kg, about 0.01 mg/kg to about 0.1 mg/kg per day or about 0.03 mg/kg to about 0.1 mg/kg per day. In other embodiments, the therapeutically effective dose ranges from about 1-10 μg/kg, about 0.03 mg/kg to about 0.1 mg/kg. In some specific, but not limiting embodiments, the therapeutically effective dose is about 0.25 μg/kg, about 0.3 μg/kg, about 0.375 μg/kg, about 0.5 μg/kg, about 0.75 μg/kg, about 1 μg/kg, about 1.5 μg/kg, about 2 μg/kg, about 3 μg/kg, about 4 μg/kg, about 4.5 μg/kg, about 5 μg/kg, about 6 μg/kg, about 7 μg/kg, about 8 μg/kg, about 9 μg/kg, about 10 μg/kg, about 15 μg/kg, about 20 μg/kg, about 25 μg/kg, about 0.01 mg/kg, about 0.03 mg/kg, about 0.05 mg/kg, or about 0.1 mg/kg per day.

It will be appreciated that the doses for the long-acting IL-15R agonist as described above may refer to either of the conjugate or the protein equivalent. In some preferred embodiments, the doses are in protein equivalents.

In embodiments, the dose level of the antibody and/or the long-acting IL-15R agonist may be reduced when accompanied by an increased dose of the antibody or the long-acting IL-15R agonist, respectively. As shown in Example 14, a reduction in dose level for the antibody treatment or the long-acting IL-15R agonist was compensated by an associated increase in dose level of the IL-15R agonist or antibody, respectively in a mouse tumor model. Thus, administration of a low dose of the antibody in combination with a high dose of the long-acting IL-15R agonist as well as administration of high dose of the antibody in combination with a low dose of the long-acting IL-15R agonist were both effective in reducing Daudi cell numbers in the bone marrow of a mouse tumor model.

With reference to the doses referenced in the examples herein, one of ordinary skill in the art could convert the animal doses (e.g. mouse) to a corresponding dose in humans using conversions as known in the art (e.g. Nair et al., J. Basic and Clin. Pharmacy (2016) 7:27-31).

The unit dosage of any given antibody and/or conjugate (again, preferably provided as part of a pharmaceutical preparation) can be administered in a variety of dosing schedules depending on the judgment of the clinician, needs of the patient, and so forth. The specific dosing schedule will be known by those of ordinary skill in the art or can be determined experimentally using routine methods. Exemplary dosing schedules include, without limitation, administration once daily, three times weekly, twice weekly, once weekly, twice monthly (e.g. q/14 days), once monthly (e.g. q/30 or 31 days, q/28 days or q/21 days), and any combination thereof. It will be appreciated that the dosing schedule may be adjusted as needed, e.g. administration once weekly for a period of time and then adjusted to a shorter or longer schedule as needed. Once a desired clinical endpoint has been achieved, dosing of the composition is halted or reduced. In some embodiments, the unit dose of any given conjugate may be administered once to provide sustained effect.

The actual dose of the antibody and/or the long-acting IL-15 receptor agonist to be administered will vary depending upon the age, weight, and general condition of the subject as well as the severity of the condition being treated, the judgment of the health care professional, and conjugate being administered. Therapeutically effective amounts are known to those skilled in the art and/or are described in the pertinent reference texts and literature.

The combination of antibody and long-acting IL-15 receptor agonist is suitable for administering to a patient suffering from a cancer or a tumor. The method comprises administering to a patient a therapeutically effective amount of an antibody directed against a tumor antigen and, generally parenterally, a therapeutically effective amount of the long-acting IL-15 receptor agonist (preferably provided as part of the same or different pharmaceutical compositions).

The antibody and/or the long-acting IL-15 receptor agonist may be administered by any suitable means as known in the art. In some embodiments, one or both of the antibody and the long-acting IL-15 receptor agonist may be administered parenterally which includes subcutaneous, intravenous, intra-arterial, intraperitoneal, intracardiac, intrathecal, and intramuscular injection, as well as infusion injections. Suitable formulation types for parenteral administration include ready-for-injection solutions, dry powders for combination with a solvent prior to use, suspensions ready for injection, dry insoluble compositions for combination with a vehicle prior to use, and emulsions and liquid concentrates for dilution prior to administration, among others. In some particular embodiments, the antibody and/or the long-acting IL-15 receptor agonist is provided in a formulation suitable for intravenous administration, and is administered intravenously. In some other embodiments, the antibody and/or the long-acting IL-15 receptor agonist is provided in a formulation suitable for subcutaneous administration, and is administered subcutaneously. In some additional embodiments, one or both of the antibody and the long-acting IL-15 receptor agonist is administered intratumorally. Other modes of administration are also contemplated, such as pulmonary, nasal, buccal, rectal, sublingual and transdermal.

The method of administering the long-acting IL-15 receptor agonist (e.g., provided as part of a pharmaceutical composition) can optionally be conducted so as to localize the agonist to a specific area. For example, liquid, gel and solid formulations comprising the agonist may also be surgically implanted in a diseased area (such as in a tumor, near a tumor, in an inflamed area, and near an inflamed area). Conveniently, organs and tissue can also be imaged in order to ensure the desired location is better exposed to the conjugate.

As described herein, one aspect of the present disclosure provides a method for that is useful for (among other things) treating a patient suffering from a condition that is responsive to treatment with either or both of the compounds. For example, patients may be responsive to the individual agents alone as well as the combination, but are more responsive to the combination. By way of further example, patients may be non-responsive to one of the individual agents, but are responsive to the combination. By way of still further example, patients may be non-responsive to either of the individual agents alone, but are responsive to the combination.

As used herein in reference to treatment of a subject having cancer, the terms “treatment,” “treat,” and “treating” are meant to include the full spectrum of intervention for the cancer from which the subject is suffering, such as administration of the combination to alleviate, slow, stop, or reverse one or more symptoms of the cancer or to delay the progression of the cancer even if the cancer is not actually eliminated. Treatment can include, for example, a decrease in the severity of a symptom, the number of symptoms, or frequency of relapse, e.g., the inhibition of tumor growth, the arrest of tumor growth, or the regression of already existing tumors. Further, the term “treating cancer” is not intended to be an absolute term, and may include, for example, reducing the size of a tumor or number of cancer cells, causing a cancer to go into remission, or preventing growth in size or number of cancer cells, and the like. In some circumstances, treatment in accordance with the instant disclosure leads to an improved prognosis.

For example, an improvement in the cancer or a cancer-related disease may be characterized as a complete or partial response. “Complete response” refers to an absence of clinically detectable disease with normalization of any previously abnormal radiographic studies, bone marrow, and cerebrospinal fluid (CSF) or abnormal monoclonal protein measurements. “Partial response” refers to at least about a 10%, 20%, 30%, 40%, 50%, 60%, 70%, 80%, or 90% decrease in all measurable tumor burden (i.e., the number of malignant cells present in the subject, or the measured bulk of tumor masses or the quantity of abnormal monoclonal protein) in the absence of new lesions. The term “treatment” contemplates both a complete and a partial response.

The terms “cancer” and “cancerous” refer to or describe the physiological condition in mammals that is typically characterized by unregulated cell growth.

“Tumor” and “solid tumor” as used herein, refer to all lesions and neoplastic cell growth and proliferation, whether malignant or benign, and all pre-cancerous and cancerous cells and tissues.

As used herein, the term “enhanced” or “enhancing”, for example, in the context of an enhanced response, refers to a subject's or tumor cell's improved ability to respond to treatment, e.g., as disclosed herein, when compared to a given baseline or reference therapy. For example, an enhanced response may comprise an increase in responsiveness of at least 5%, 10%, 15%, 20%, 25%, 30%, 35%, 40%, 45%, 50%, 55%, 60%, 65%, 70%, 75%, 80%, 85%, 90%, 95% or 98% or more, based upon any one or more indicators of responsiveness to treatment. As used herein, “enhanced” or “enhancing” can also refer to enhancing the number of subjects who favorably respond to treatment, e.g., when compared to a given basis for such comparison.

Exemplary conditions are cancers, such as, for example, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, colon cancer, prostate cancer, squamous cell cancer, basal cell cancer, head and neck cancer, adenocarcinoma, sweat gland cancer, sebaceous gland cancer, papillary cancer, papillary adenocarcinomas, cystadenocarcinoma, medullary cancer, bronchogenic cancer, renal cell cancer, hepatoma, bile duct cancer, choriocarcinoma, seminoma, embryonal cancer, Wilms' tumor, cervical cancer, testicular cancer, lung cancer, small cell lung cancer, non-small cell lung cancer, bladder cancer, colorectal cancer, epithelial cancer, glioma, astrocytoma, medulloblastoma, craniopharyngioma, ependymoma, pinealoma, hemangioblastoma, acoustic neuroma, oligodendroglioma, meningioma, melanoma (including, for example, uveal melanoma, mucosal melanoma, and leptomeningeal melanoma), neuroblastoma, retinoblastoma, squamous cell carcinoma of the head and neck, leukemias, and lymphomas. Further exemplary conditions are hematological malignancies including without limitation non-Hodgkin lymphoma and multiple myeloma.

In one particular method, the monoclonal antibody and the long-acting IL-15 receptor agonist are used to treat a hematological malignancy such as a leukemia or lymphoma. In yet another method, the monoclonal antibody and the long-acting IL-15 receptor agonist are used to treat a solid cancer. In some embodiments, the solid cancer is, without limitation, selected from colon cancer, colorectal cancer, pancreatic cancer, breast cancer, ovarian cancer, prostate cancer, head and neck cancer, renal cell cancer, cervical cancer, and small cell lung cancer, non-small cell lung cancer.

In some embodiments, the combination of an antibody as described herein and a long-acting IL-15 receptor agonist (or related composition) is effective, when administered at a therapeutically effective dose to a subject, to stimulate NK activation and/or proliferation to an extent greater than that observed for either agent administered singly and at an equivalent dose, e.g., in a suitable animal model. In some embodiments, the combination of a monoclonal antibody as described herein and a long-acting IL-15 receptor agonist or composition is effective when administered at a therapeutically effective dose to a subject, to provide an increased tumor clearance than that observed for either agent administered singly, and at an equivalent dose, e.g., when evaluated in a suitable animal model. In some embodiments, the combination of a monoclonal antibody as described herein and a long-acting IL-15 receptor agonist or composition is effective when administered at a therapeutically effective dose to a subject to provide increased and/or long-term survival of the subject, e.g., an increase in survival, or a greater degree of tumor regression, than that observed for either agent administered singly, and at an equivalent dose, e.g., when evaluated in a suitable animal model. In some embodiments, the combination of an antibody as described herein and a long-acting IL-15 receptor agonist or composition is effective when administered at a therapeutically effective dose to a subject to provide an increase in the activity of one or both of the long-acting IL-15 receptor agonist and the antibody, especially within a tissue compartment such as, e.g., bone marrow. In some embodiments, the combination of an antibody as described herein and a long-acting IL-15 receptor agonist or composition is effective when administered at a therapeutically effective dose to a subject to provide enhanced degranulation of NK cells and/or increased NK cell viability. In embodiments, administration of an antibody as described herein and a long-acting IL-15 receptor agonist or composition is effective when administered at a therapeutically effective dose to a subject to treat a cancer that is resistant to treatment by the monoclonal antibody alone.

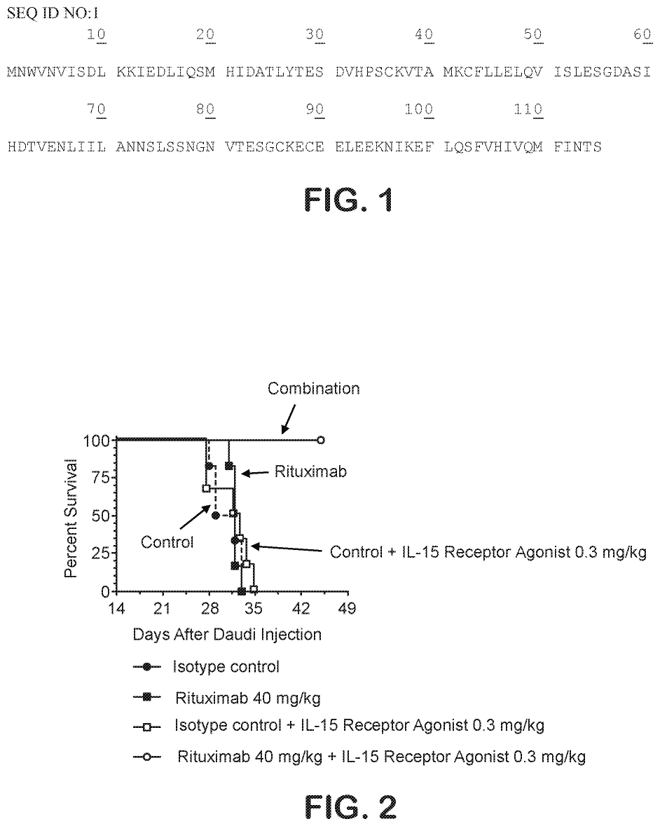

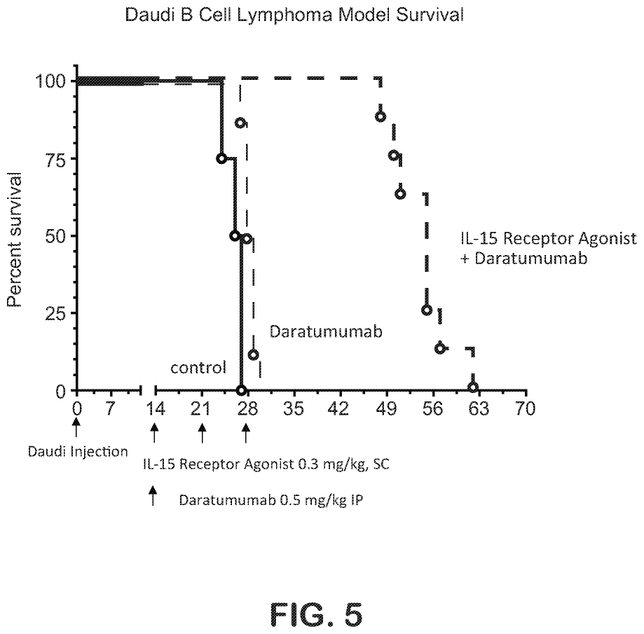

Notable findings associated with the combination therapy as described herein are highlighted below and further detailed in the illustrative examples included herein. In an exemplary Daudi B cell lymphoma mouse model as described in Example 1, administration of an exemplary long-acting IL-15 receptor agonist and the anti-CD20 monoclonal antibody rituximab was effective to prolong survival as compared to administration of either the long-acting IL-15 receptor agonist or the antibody alone. As shown in FIG. 1, mice treated with a long-acting IL-15 receptor agonist and the anti-CD20 antibody, rituximab, had 100% survival after about 45 days. Mice treated with the isotype control or rituximab alone had 0% survival after about 30 days. The mice treated with an isotype control and the long-acting IL-15 receptor agonist had 0% survival after about 35 days. In a further exemplary mouse model as described in Example 2, administration of a long-acting IL-15R agonist (e.g. mono(methoxyPEG-N-butanamide)interleukin-15) and the anti-CD38 monoclonal antibody, daratumumab, was effective to prolong survival as compared to administration of either the long-acting IL-15 receptor agonist or the antibody alone. As shown in FIG. 5, mice treated with an illustrative long-acting IL-15 receptor agonist (mono(methoxyPEG-N-butanamide)interleukin-15) and the anti-CD38 antibody, daratumumab, had a median survival of 55 days (post tumor injection) as compared to 28.5 days for the antibody single agent. As described in Example 13, administration of a long-acting IL-15R agonist (e.g., mono(methoxyPEG-N-butanamide)interleukin-15) and the anti-CD38 monoclonal antibody daratumumab to SCID mice led to a more than double increase in survival. In contrast administration of the long-acting IL-15R agonist and the anti-CD38 monoclonal antibody daratumumab to SCID beige mice, which have reduced NK cell functionality, had reduced increase in survival. These results indicate that the effect on NK cell functionality by administration of the long-acting IL-15R agonist and the anti-CD38 monoclonal antibody daratumumab leads to a longer survival. Examples 18 and 19 show increased survival with administration of a long-acting IL-15R agonist (e.g. mono(methoxyPEG-N-butanamide)interleukin-15) and the anti-EGFR monoclonal antibody, cetuximab, in two exemplary colorectal carcinoma mouse models (a HT-29 colorectal carcinoma cell model and an HCT-116 colorectal carcinoma xenograft model).

In some embodiments, administration of a long-acting IL-15 receptor agonist and an antibody as described herein is effective to provide an increase in the duration of survival of the subject over treatment of the long-acting IL-15 receptor agonist and/or the antibody alone. In some other embodiments, administration of a long-acting IL-15 receptor agonist and an antibody as described herein is effective to provide an increase in the duration of survival of the subject over conventional treatment. In one or more embodiments, the duration of survival is increased by at least about 1, 2, 3, 4, 5, 6, 7, 8, 9, 10, 11, 12, 18, 24 months or longer when compared to a subject treated with only one of the components of the presently described combination or a conventional therapy for the same indication. In some embodiments, the duration of survival is at least about 1, 2, 3, 4, 5 years or longer.

In some embodiments, the present combination treatment is effective to increase the progression-free survival (survival without substantial progression of the disease being treated) of the subject. In embodiments, the progression-free survival is increased by at least about 1, 2, 3, 4, 5, 6, 7, 8, 9, 10, 11, 12, 18, 24 months or longer when compared to a subject treated with only one of the components of the presently described combination or a conventional therapy for the same indication. In some embodiments, the progression-free survival is at least about 1, 2, 3, 4, 5 years or longer.

In certain embodiments, the present combination treatment is effective to increase the response rate of subjects treated with the combination. In embodiments, the present combination is effective to increase the response of treated subjects by at least about 5%, 10%, 15%, 20%, 25%, 30%, 40%, 50%, 60%, 70%, 75%, 80%, 90% or 100% when compared to a subject treated with only one of the components of the presently described combination or a conventional therapy for the same indication.

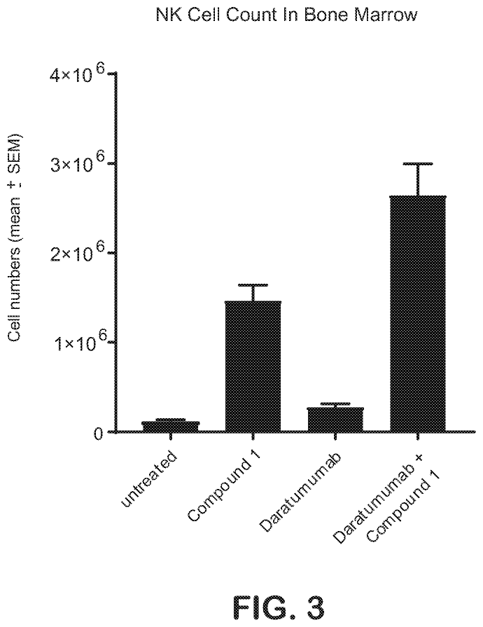

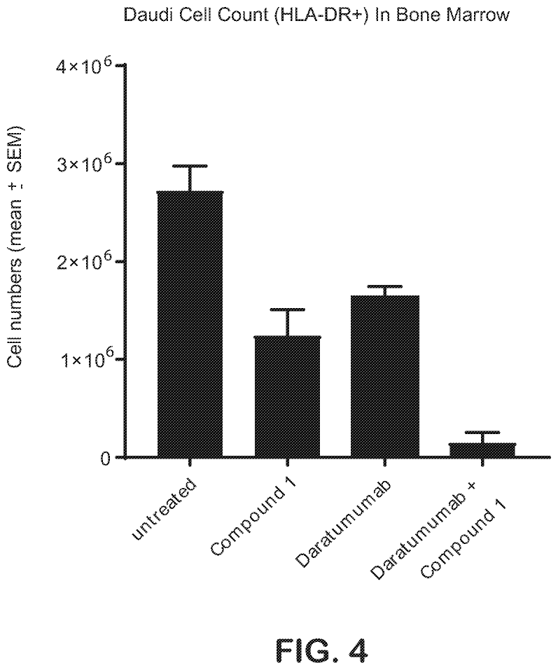

The combination therapy is also effective to promote NK cell expansion in a subject to a degree that is notably enhanced over either agent administered singly or untreated subjects. In an exemplary Daudi B cell lymphoma xenograft mouse model as described in Example 2, administration of a long-acting IL-15 receptor agonist, more particularly mono(methoxyPEG-N-butanamide)interleukin-15, and the anti-CD38 monoclonal antibody, daratumumab, resulted in significant expansion of NK cells in the bone marrow of treated mice. As seen in FIG. 3, mice treated with the long-acting IL-15 receptor agonist and the anti-CD38 antibody daratumumab exhibited a significant increase in NK cell proliferation (cell numbers) in the bone marrow as compared to untreated mice or mice treated with either agent alone. As seen in FIG. 4, mice treated with the long-acting IL-15 receptor agonist and the anti-CD38 antibody daratumumab had a significantly decreased number of B cell lymphoma cells in the bone marrow as compared to untreated mice or mice treated with either agent alone. Example 16 shows increased proliferation of NK cells in tumor tissue after administration of an exemplary long-acting IL-15R agonist (e.g., mono(methoxyPEG-N-butanamide)interleukin-15) and the anti-EGFR monoclonal antibody, cetuximab, in an exemplary FaDu mouse model for human head and neck squamous cell carcinoma.

In some embodiments, administration of a long-acting IL-15 receptor agonist and an antibody as described herein is effective to provide an increase in NK cell expansion and proliferation. In some further embodiments, the present combination is effective to increase the proliferation or expansion of NK cells of treated subjects by at least about 5%, 10%, 15%, 20%, 25%, 30%, 40%, 50%, 60%, 70%, 75%, 80%, 90% or 100% when compared to a subject treated with only one of the components of the presently described combination or a conventional therapy for the same indication. In embodiments, the present combination is effective to increase the proliferation or expansion of NK cells of treated subjects by at least about 5-fold, 10-fold, 15-fold, 20-fold, 25-fold or greater when compared to a subject treated with only one of the components of the presently described combination or a conventional therapy for the same indication.

In some embodiments, administration of a long-acting IL-15 receptor agonist and an antibody as described herein is effective to provide an increase in cancer cell depletion/decrease in cancer cell numbers. In some embodiments, the present combination is effective to increase depletion of cancer cells of treated subjects by at least about 5%, 10%, 15%, 20%, 25%, 30%, 40%, 50%, 60%, 70%, 75%, 80%, 90% or 100% when compared to a subject treated with only one of the components of the presently described combination or a conventional therapy for the same indication. In embodiments, the present combination is effective to increase the depletion of cancer cells of treated subjects by at least about 5-fold, 10-fold, 15-fold, 20-fold, 25-fold or greater when compared to a subject treated with only one of the components of the presently described combination or a conventional therapy for the same indication.

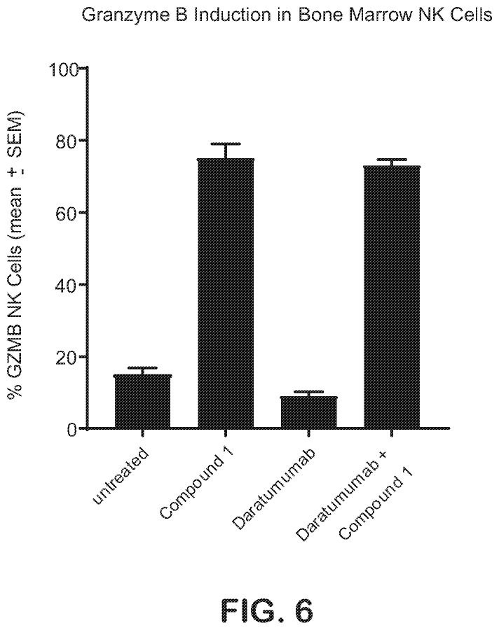

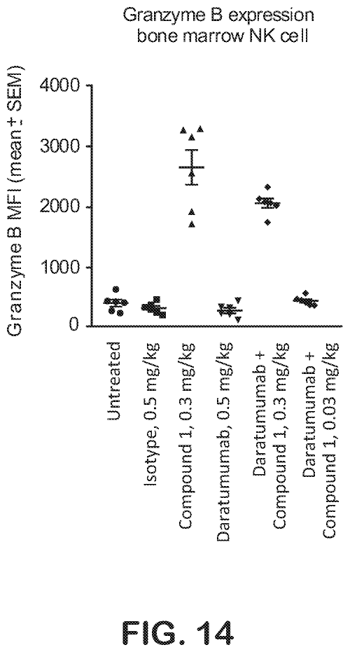

In an exemplary Daudi B cell lymphoma xenograft mouse model as described in Example 2, administration of an exemplary long-acting IL-15 receptor agonist (e.g., mono(methoxyPEG-N-butanamide)interleukin-15) and the anti-CD38 monoclonal antibody, daratumumab, resulted in a significant increase in granzyme B expression in bone marrow NK cells of treated mice. As seen in FIG. 6, a majority of mice treated with the exemplary long-acting IL-15 receptor agonist (e.g., mono(methoxyPEG-N-butanamide)interleukin-15) and the anti-CD38 antibody, daratumumab, exhibited an increase in granzyme B expression in NK cells in the bone marrow as compared to untreated mice or mice treated with the antibody alone. As further seen in FIG. 6, the combination treatment activated the function in a majority of the NK cells as shown by the increase in granzyme B (a cytotoxic protease) marker. As shown in FIG. 14 and described in Example 11, administration of an exemplary long-acting IL-15 receptor agonist (e.g., mono(methoxyPEG-N-butanamide)interleukin-15) and the anti-CD38 monoclonal antibody, daratumumab, was effective to increase granzyme B expression for NK cells in the bone marrow on a per cell basis when compared to untreated mice or treatment with the antibody alone. In an exemplary FaDu human head and neck squamous cell carcinoma mouse model as described in Example 16, administration of an exemplary long-acting IL-15 receptor agonist (e.g., mono(methoxyPEG-N-butanamide)interleukin-15) and the anti-EGFR monoclonal antibody, cetuximab, was effective to increase granzyme B expression in NK cells present in blood and tumor samples as compared to untreated mice.

In some embodiments, administration of a long-acting IL-15 receptor agonist and an antibody as described herein is effective to provide an increase in granzyme B expression in NK cells. In one or more embodiments, the present combination is effective to increase granzyme B expression in NK cells of treated subjects by at least about 5%, 10%, 15%, 20%, 25%, 30%, 40%, 50%, 60%, 70%, 75%, 80%, 90% or 100% when compared to a subject treated with only one of the components of the presently described combination or a conventional therapy for the same indication. In embodiments, the present combination is effective to increase granzyme B expression in NK cells of treated subjects by at least about 5-fold, 10-fold, 15-fold, 20-fold, 25-fold or greater when compared to a subject treated with only one of the components of the presently described combination or a conventional therapy for the same indication.

In an exemplary mouse model as described in Example 10, administration of an exemplary long-acting IL-15 receptor agonist (mono(methoxyPEG-N-butanamide)interleukin-15) and the anti-CD38 monoclonal antibody, daratumumab, was effective to increase the fraction of NK cells expressing the cell surface CD16 receptor as compared to administration of either the long-acting IL-15 receptor agonist or the antibody alone. In a further exemplary colorectal carcinoma mouse model as described in Example 15, administration of a long-acting IL-15 receptor agonist and the anti-EGFR monoclonal antibody cetuximab was effective to increase CD16 cell surface expression in blood and tumor samples as compared to untreated mice. In some embodiments, the long-acting IL-15R agonists provide enhanced surface expression of low affinity Fc receptor CD16 to provide a further immunotherapeutic effect when used in combination with antibodies directed towards CD16 and having ADCC mechanism of action.

In a third aspect, a kit comprising an antibody and long-acting IL-15 receptor agonist as described above is provided herein. The kit may further include instructions for use as well as, optionally, any medical supplies or devices as needed for administration of the antibody and the long-acting IL-15 receptor agonist.

It is to be understood that while the methods, combinations, kits, etc. have been described in conjunction with the preferred specific embodiments thereof, that the foregoing description as well as the examples that follow are intended to illustrate and not limit the scope of the disclosed methods, combinations, kits, etc. Other aspects, advantages and modifications within the scope of this disclosure will be apparent to those of ordinary skill in the relevant art.

All articles, books, patents and other publications referenced herein are hereby incorporated by reference in their entireties. In the event of an inconsistency between the teachings of this specification and the art incorporated by reference, the meaning of the teachings and definitions in this specification shall prevail (particularly with respect to terms used in the claims appended herein). For example, where the present application and a publication incorporated by reference defines the same term differently, the definition of the term shall be preserved within the teachings of the document from which the definition is located.

### EXAMPLES

It is to be understood that the foregoing description as well as the examples that follow are intended to illustrate and not limit the scope of the methods, combinations, kits, etc. provided herein. Other aspects, advantages and modifications will be apparent to those skilled in the art to which this disclosure pertains.

In the following examples, efforts have been made to ensure accuracy with respect to numbers used (e.g., amounts, temperatures, etc.) but some experimental error and deviation should be taken into account. Unless indicated otherwise, temperature is in degrees C. and pressure is at or near atmospheric pressure at sea level. Each of the following examples is considered to be instructive to one of ordinary skill in the art for carrying out one or more of the embodiments described herein.

### Materials and Methods

The exemplary long-acting IL-15 receptor agonist used in the following examples, unless otherwise indicated, is mono(methoxyPEG-N-butanamide)_{40 kD} interleukin-15, generally described by the following structure:

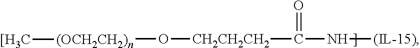

where n has a value of about 909 (such that the polyethylene glycol portion of the molecule has a weight average molecular weight of about 40 kilodaltons), also referred to as [mPEG_{40 kD}-CH₂CH₂CH₂C(O)NH]-IL-15. This exemplary long-acting IL-15 receptor agonist is described herein as Conjugate 1 or Compound 1, which terms are used interchangeably in the following examples. Preparation of mono(methoxyPEG-N-butanamide)interleukin-15 is described in Example 1 of PCT Application No. PCT/US2018/032817 (International Publication No. WO 2018/213341). Doses and concentrations in these examples are based on IL-15 protein content.

### SDS-PAGE Analysis

Samples may be analyzed by sodium dodecyl sulfate-polyacrylamide gel electrophoresis (SDS-PAGE) using Invitrogen gel electrophoresis system (XCell SureLock Mini-Cell). Samples are mixed with sample buffer and the prepared samples are loaded onto a NuPAGE Novex precast gel and run for approximately thirty minutes.

### RP-HPLC Analysis

Reversed-phase chromatography (RP-HPLC) analysis may be performed on an Agilent 1200 HPLC system (Agilent). Samples may be analyzed using a Poroshell 300SB-C3 column (2.1×75 mm, Agilent) at 60° C. The mobile phases may be 0.1% TFA/H2O (A) and 0.1% TFA/CH3CN (B). The flow rate for the column may be 0.5 ml/min. Eluted protein and PEG-protein conjugates are detected using UV at 280 nm.

### Bioassays

### Potency Assay (Mouse T Cell) Based on STAT5 Phosphorylation in CTLL-2 Cells

In the phospho-STAT5 assay following receptor binding, downstream cell signaling can then activate Signal Transducer and Activator of Transcription 5 (STAT5) through phosphorylation to promote gene expression to induce cell proliferation. The activation of phosho-STAT5 is measured in an appropriate cell line, using the phospho-STAT5/total STAT5 multiplexed assay (Meso Scale Discovery, MD) in response to sample and reference treatment for ˜10 minutes and as described in PCT Application No. PCT/US2018/032817, which is incorporated herein by reference.

### HuPBMC-pStat5 Assay

The potency of IL-15 or long-acting IL-15 receptor agonists on various cells may be determined by phospho-STAT5(Y694) dose response assay as described further in PCT Application No. PCT/US2018/032817.

### Example 1

### Evaluation of Survival in a Daudi B Cell Lymphoma Xenograft Model Following Administration of a Long-Acting IL-15 Receptor Agonist and an Exemplary Anti-Cd-20 Monoclonal Antibody, Rituximab

At day 0, SCID mice were intravenously injected with 1×10⁷ Daudi cells. The mice were divided into four groups. On days 14 and 17 post-Daudi cell injection, the groups were treated by injection as follows: Group 1: isotype control antibody, Group 2: 40 mg/kg IP rituximab, Group 3: 0.3 mg/kg mono(methoxyPEG-N-butanamide)_{40 kD} interleukin-15 (a long-acting IL-15 receptor agonist), Group 4: 40 mg/kg IP rituximab and 0.3 mg/kg SC mono(methoxyPEG-N-butanamide)_{40 kD} interleukin-15 (combination).

The survival rate was determined by measuring the onset of hind limb paralysis as a surrogate parameter. The results are illustrated in FIG. 2.

As seen in FIG. 2, the combined administration of rituximab and mono(methoxyPEG-N-butanamide)interleukin-15 (indicated in FIG. 2 as “IL-15 receptor agonist”) provided a significant increase in long-term survival when compared to administration of either component when administered as a single agent therapy (see horizontal line marked “combination”). This result demonstrates the ability of the long-acting receptor agonist, mPEG-BA-IL-15, when administered in combination with an illustrative anti-CD-20 monoclonal antibody such as rituximab, to significantly enhance survival times in mice when evaluated in an in vivo lymphoma model.

### Example 2

### Evaluation of Cell Counts and Granzyme B Expression in Bone Marrow in a Daudi B Cell Lymphoma Xenograft Model Following Administration of a Long-Acting IL-15 Receptor Agonist and an Exemplary Anti-CD38 Monoclonal Antibody, Daratumumab

SCID mice (N=6/group) were intravenously inoculated with 1×10⁷ Daudi cells on Day 0 and treated with a single dose of the anti-human CD38 antibody, daratumumab (0.5 mg/kg IP, 14 days following Daudi cell inoculation) and two doses of mono(methoxyPEG-N-butanamide)interleukin-15 (0.3 mg/kg SC, one dose at each of 14 and 21 days after inoculation). Mice were also treated with the single agent therapies: daratumumab (0.5 mg/kg IP, 14 days following Daudi cell inoculation) or two doses of mono(methoxyPEG-N-butanamide)_{40 kD} interleukin-15 (0.3 mg/kg SC, 14 and 21 days after inoculation).

NK cell expansion and activation and lymphoma depletion in the bone marrow were assessed three days following the second dose of mono(methoxyPEG-N-butanamide)_{40 kD} interleukin-15 (day 24) by flow cytometry as shown in FIGS. 3 and 4, respectively. As shown in FIG. 3, the combination of mono(methoxyPEG-N-butanamide)interleukin-15 and daratumumab resulted in a significant expansion of NK cells in bone marrow of treated mice in comparison to mice treated with mono(methoxyPEG-N-butanamide)_{40 kD} interleukin-15 and daratumumab as single agent therapies (p=0.0026 and p<0.0001, respectively) and compared to untreated control (p<0.0001). (One-way ANOVA, Tukey's multiple comparison test). A 19-fold increase in NK cell count was observed for the combination therapy versus a 10-fold increase for mono(methoxyPEG-N-butanamide)_{40 kD} interleukin-15 mono-therapy, demonstrating a synergism between the exemplary monoclonal antibody and mono(methoxyPEG-N-butanamide)interleukin-15 inside the bone marrow tissue.

As illustrated in FIG. 4, mono(methoxyPEG-N-butanamide)interleukin-15 in combination with daratumumab significantly improves B cell lymphoma depletion compared to mono(methoxyPEG-N-butanamide)interleukin-15 and daratumumab single agent treatments (p=0.02 and p=0.001, respectively) and compared to untreated control (p<0.0001). (One-way ANOVA, Tukey's multiple comparison test).

Granzyme B expression was measured in bone marrow NK cells as shown in FIG. 6. As illustrated therein, mono(methoxyPEG-N-butanamide)interleukin-15 is effective to control NK cell dynamics even in highly specialized immune compartments, and is effective, either alone or in combination with a monoclonal antibody such as daratumumab, to increase granzyme B expression in the bone marrow of treated mice. Thus, the combination of mono(methoxyPEG-N-butanamide)interleukin-15 with a tumor binding antibody such as daratumumab is effective not only to significantly expand NK cell numbers in bone marrow but to also activate the killing function in a majority (>70%) of these NK cells, as shown by the granzyme B (a cytotoxic protease) marker in FIG. 6.

These results further illustrate that mono(methoxyPEG-N-butanamide)interleukin-15 is effective to substantially enhance NK-cell mediated ADCC when administered with an anti-tumor antibody.

### Example 3

### Evaluation of Survival in a Daudi B Cell Lymphoma Xenograft Model Following Administration of a Long-Acting IL-15 Receptor Agonist and an Anti-CD38 Monoclonal Antibody, Daratumumab

SCID mice (N=8/group) inoculated intravenously with 1×10⁷ Daudi B cell lymphoma cells were treated with a single dose of daratumumab (0.5 mg/kg, IP, 14 days after inoculation) and a total of three doses of mono(methoxyPEG-N-butanamide)_{40 kD} interleukin-15 (0.3 mg/kg, SC, administered 14, 21 and 28 days after tumor inoculation). Also evaluated were untreated mice as a control and mice treated with daratumumab as a single agent therapy. Survival of tumor-inoculated mice was measured by body condition scoring as endpoint marker. Results are provided graphically in FIG. 5.

As shown in FIG. 5, the mono(methoxyPEG-N-butanamide)interleukin-15 combination with daratumumab significantly increases median survival compared to daratumumab single agent treatment (p<0.05, Log-Rank test). The median survival for the group treated by the combination therapy was 55 days post tumor injection, while the median survival for the daratumumab single agent treatment group was 28.5 days.

### Example 4

### NK Cell Activating and Inhibitory Receptor Cell Surface Expression Modulation Following Treatment with Tumor Targeting Therapeutic Antibodies and a Long-Acting IL-15 Receptor Agonist

Balb/c mice bearing Daudi B cell lymphoma tumors in the bone marrow (n=6/group) were intraperitoneally administered a single 0.5 mg/kg dose of the human CD38 protein targeting clinical antibody daratumumab (DARZALEX®, Janssen Biotech) or a nonspecific control antibody with a matching isotype on day 14 after Daudi cell intravenous inoculation. Additional groups were administered mono(methoxyPEG-N-butanamide)_{40 kD} interleukin-15 (Conjugate 1) subcutaneously at a dose of 0.3 mg/kg as a single agent on days 14 and 21 after Daudi cell inoculation, or administered a combination treatment of Conjugate 1 administered subcutaneously at doses of 0.03 mg/kg or 0.3 mg/kg on days 14 and 21 after Daudi cell inoculation and daratumumab administered intraperitoneally at a dose of 0.5 mg/kg on day 14 after Daudi cell inoculation.

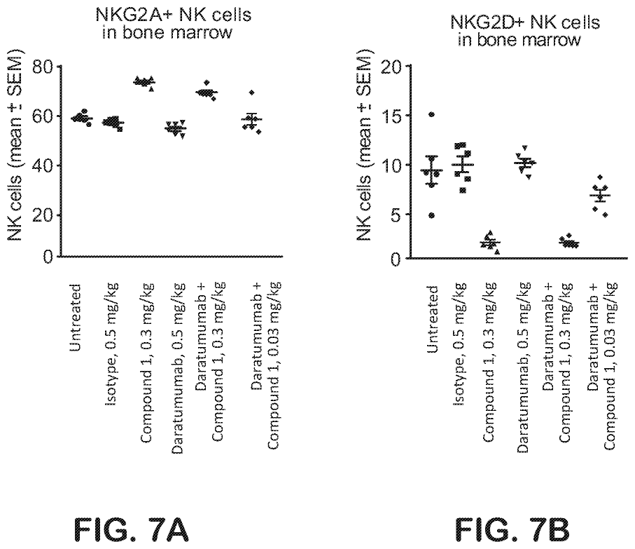

Three days following the second Conjugate 1 administration (day 24 after Daudi inoculation), bone marrow was collected and the NK cell fraction was measured by flow cytometry in total bone marrow. Expression of the cell surface activating receptor NKG2D and the inhibitory checkpoint receptor NKG2A are shown in FIGS. 7A and 7B, respectively.

Results are provided in Table 1 and FIGS. 7A-7B. All groups that received therapeutic dose of Conjugate 1 as a single agent or in combination with daratumumab showed increased fraction of NK cells that expressed NKG2A on the cell surface (FIG. 7A) and reduction of the fraction of NK cells that expressed NKG2D on the cell surface (FIG. 7B) when compared to all of the treatments not including Conjugate 1. The respective increase in inhibitory checkpoint receptor activation and decrease in activating receptor expressing NK cells was indicative of NK cell activation by Conjugate 1 in the presence of therapeutic tumor targeting antibody as exemplified by daratumumab in this example.

TABLE 1
NKG2A and NKG2D surface expression modulation
by Conjugate 1 and daratumumab.

| Treatment of | | |
|---|---|---|
| Daudi B cell | | |
| lymphoma bearing | % NK cells expressing | % NK cells expressing |
| mice | NKG2A (mean, n = 6) | NKG2D (mean, n = 6) |
| No treatment | 59.08 | 9.480 |
| Isotype control | 57.13 | 10.04 |
| antibody | | |
| Daratumumab | 54.83 | 10.22 |
| Conjugate 1 | 73.70 | 2.157 |
| 0.3 mg/kg | | |
| Conjugate 1 | 69.47 | 2.138 |
| 0.3 mg/kg and | | |
| daratumumab | | |
| Conjugate 1 | 58.60 | 6.890 |
| 0.03 mg/kg and | | |
| daratumumab | | |

### Example 5

### Induction of Human NK Cell Proliferation Following Treatment with a Long-Acting IL-15 Receptor Agonist and an Immunoglobulin

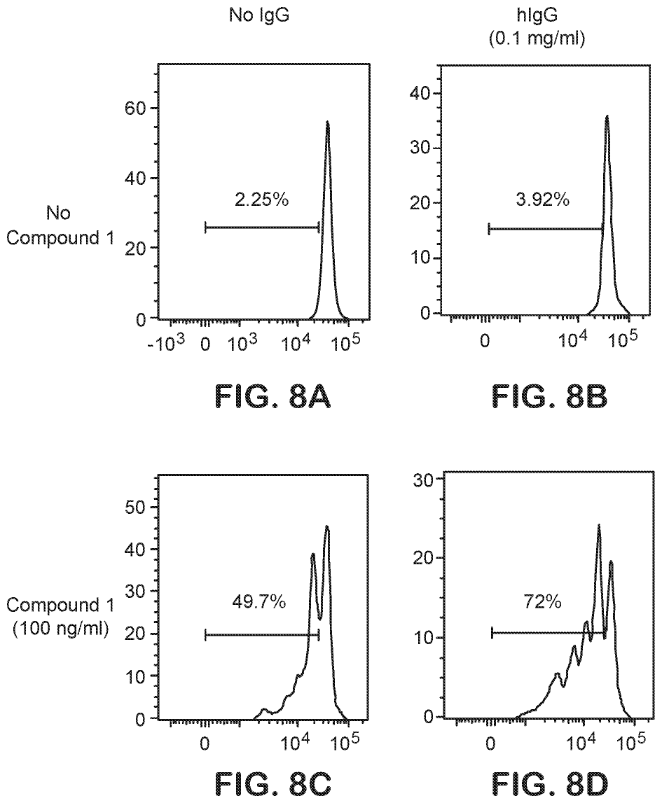

NK cell proliferation within a human peripheral blood mononuclear cell (PBMCs) labeled with CFSE preparation was induced in vitro. The CFSE labeled PBMCs were incubated for 5 days in vitro in the presence of (i) mono(methoxyPEG-N-butanamide)_{40 kD} interleukin-15 (Conjugate 1) at a concentration of 100 ng/ml; (ii) on cell culture plastic that had been prior coated overnight with human total immunoglobulin G (hIgG) solution at a concentration of 0.1 mg/ml (iii) on cell culture plastic that had been prior coated overnight with human total immunoglobulin G (hIgG) solution at a concentration of 0.1 mg/ml and in the presence of Conjugate 1 at a concentration of 100 ng/ml; or (iv) on regular cell culture plastic. After the incubation period, cell proliferation as indicated by the decrease of CFSE in the cells that had undergone cell doublings was measured with flow cytometry (FIGS. 8A-8D) measuring CFSE levels in CD56+ human NK cells within the PBMCs preparation in each of the four incubation conditions.

Results are provided in Table 2 and FIGS. 8A-8D. The data shows that only 2.25% of the NK cells proliferated when neither Conjugate 1 nor hIgG were exposed to the PBMCs. 3.92% of the NK cells proliferated in the presence of hIgG alone. Conjugate 1 exposure resulted in proliferation of 49.7% of the NK cells. However, when PBMCs were exposed to both hIgG and Conjugate 1, 72% of the NK cells proliferated indicating that hIgG and Conjugate 1 combination treatment caused significantly more cells to proliferate than the sum of hIgG and Conjugate 1 single agent treatments alone.

TABLE 2
Human NK cells proliferation in the presence
of Conjugate 1 and hIgG in vitro.

| PBMCs treatment | % CD56+ NK cells proliferating |
|---|---|
| No Conjugate 1, no hIgG | 2.25% |
| hIgG | 3.92% |
| Conjugate 1 | 49.7% |
| hIgG and Conjugate 1 | 72% |

### Example 6

### Functional Activation of Human NK Cells Following Administration of a Long-Acting IL-15 Receptor Agonist and an Immunoglobulin

Functional activation of human NK cells within a human peripheral blood mononuclear cells (PBMCs) preparation was induced in vitro. PBMCs from two donors were incubated overnight in the presence of (i) mono(methoxyPEG-N-butanamide)_{40 kD} interleukin-15 (Conjugate 1) at a concentration of 1 μg/ml; (ii) on cell culture plastic that had been prior coated overnight with human total immunoglobulin G (hIgG) solution at a concentration of 0.1 mg/ml (iii) on cell culture plastic that had been prior coated overnight with human total immunoglobulin G (hIgG) solution at a concentration of 0.1 mg/ml and in the presence of Conjugate 1 at a concentration of 1 μg/ml; or (iv) on regular cell culture plastic.

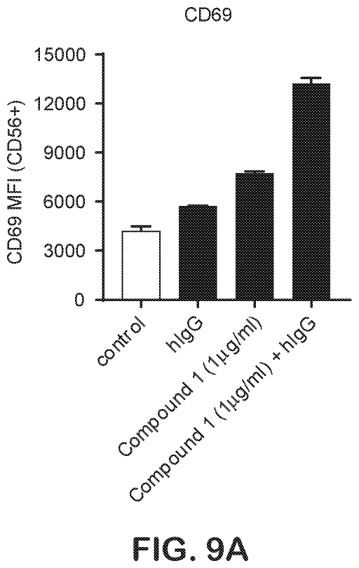

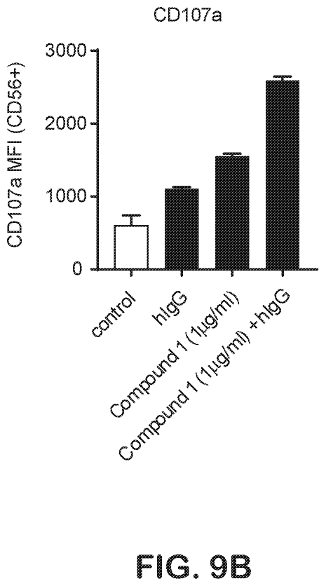

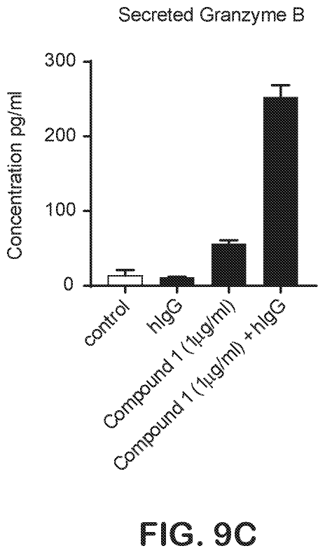

After the incubation period, NK cell activation was measured with flow cytometry by measuring the increase of median fluorescence intensity (MFI) signal of CD69 (FIG. 9A) and CD107a (FIG. 9B) detecting antibodies on the surface of CD56+ human NK cells within the PBMCs. Functional activation of NK cells was assessed by calculating the fold-change in MFI when comparing control condition where the cells were exposed to neither hIgG nor Conjugate 1. Fold-change increase in activation markers MFI indicated increase of these marker proteins on individual NK cells that indicated functional activation of the NK cells. Secreted granzyme B concentration also indicative of NK cell activation in the culture media at the end of the experiment was measured by ELISA (FIG. 9C).

Results are provided in Table 3 and FIGS. 9A-9C. The data shows that CD69 MFI on NK cells was increased by 1.4-fold in the presence of hIgG. Conjugate 1 exposure resulted in CD69 MFI increase of 1.8-fold. However, when PBMCs were exposed to both hIgG and Conjugate 1, the CD69 MFI increased by 3.2-fold on NK cells, more than each treatment individually. CD107a MFI on NK cells was increased by 1.8-fold in the presence of hIgG. Conjugate 1 exposure resulted in CD107a MFI increase of 2.6-fold. However, when PBMCs were exposed to both hIgG and Conjugate 1, the CD107a MFI increased by 4.3-fold on NK cells, more than each individual treatment. Secreted Granzyme B concentration was not increased in the presence of hIgG compared to control conditions. Conjugate 1 exposure resulted in an increase in Granzyme B concentration in the culture media of 4.1-fold. However, when PBMCs were exposed to both hIgG and Conjugate 1, the Granzyme B concentration increased by 18.7-fold in the culture media, significantly more than each treatment individually.

TABLE 3
Human NK cells activation in the presence of Conjugate 1 and hIgG in vitro.

| | CD56+ NK cells | CD56+ NK cells | Granzyme B |
|---|---|---|---|
| | mean (n = 2) | mean (n = 2) | concentration |
| | CD69 MFI and | CD107a MFI and | in pg/ml and |
| | fold-change | fold-change | fold-change |
| PBMCs | relative to no | relative to no | relative to no |
| treatment | stimulation control | stimulation control | stimulation control |
| No Conjugate 1, | 4197, N/A | 601.5, N/A | 13.5, N/A |
| no hIgG | | | |
| hIgG | 5731, 1.4 | 1104, 1.8 | 11, 0.8 |
| Conjugate 1 | 7749, 1.8 | 1553, 2.6 | 56, 4.1 |
| hIgG and | 13232, 3.2 | 2594, 4.3 | 252.6, 18.7 |
| Conjugate 1 | | | |

### Example 7

### In Vitro Signaling of Long-Acting IL-15 Receptor Agonist and Effect on Proliferation

KHYG-1 cells were cultured in RPMI-1640 medium supplemented with 20% FBS, 2 mM L-glutamine, and 20 ng/mL IL-2 (Complete growth medium) in a humidified incubator at 37° C. and 5% CO₂.

One day prior to treatment with test compounds, cells were seeded at a density of 500,000 viable cells/ml in Complete growth medium and incubated overnight under normal growth conditions. On the day of the assay, KHYG-1 cells were washed 3 times with DPBS, resuspended in assay medium (RPMI-1640 medium supplemented with 20% FBS, 2 mM L-glutamine), and incubated for 4-5 hours at 37° C., 5% CO₂. Cells were adjusted to the appropriate density (8×10⁵ cells/mL) in assay medium and 50,000 cells/well were aliquoted into wells of a 96-well plate (View-Plate 96, Perkin-Elmer).

### Evaluation of Phosphorylation of STAT5

KHYG-1 cells were stimulated with IL-15 or a long-acting IL-15 receptor agonist (mono(mPEG-butanamide)IL-15) followed by incubation at 37° C., 5% CO₂ for 10 minutes. Treated cells were lysed on ice.

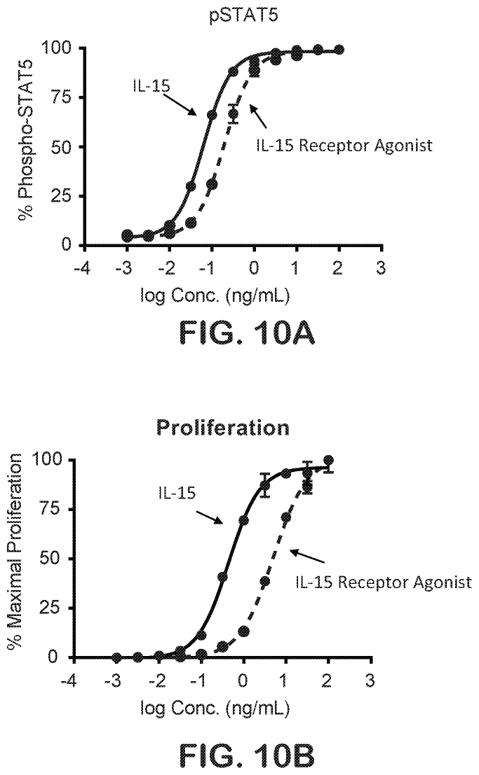

Detection of phospho-STAT5 protein levels in cell lysates was performed using the Phospho(Tyr694)/Total STAT5a,b Assay Whole Cell Lysate kit (MSD product #K15163D). Relative light unit (RLU) signal corresponding to phosphor-STAT5 was collected using a Sector® Imager 2400 plate reader (MSD) with the results provided in FIG. 10A. IL-15 and long-acting IL-15 receptor agonist both produced dose-dependent phosphorylation of STAT5 in the human NK cell line KHYG-1, showing the compounds are effective in in vitro signaling. The long-acting IL-15 receptor agonist induced phosphorylation of STAT5, producing maximal percent phosphorylation of STAT5 that was similar to IL-15, albeit with a ˜3-fold less potency. This data suggests that a long-acting IL-15 receptor agonist can effectively engage and activate downstream signaling in the KHYG-1 cell line.

### Evaluation of Cell Proliferation

Stimulation of KHYG-1 cells was initiated by the transfer of IL-15 or a long-acting IL-15 receptor agonist (mono(mPEG-butanamide)IL-15) to duplicate wells containing cells, followed by 48 hours incubation at 37° C., 5% CO₂.

Cell growth in each well was measured by monitoring ATP levels using CellTiter-Glo 2.0, following the manufacturer's instructions. The effect of the long-acting IL-15 receptor agonist on KHYG-1 cell proliferation was compared to IL-15 following 48-hour treatment using CellTiter-Glo 2.0 with the results shown in FIG. 10B. As seen in the figure, the maximal proliferative response for the long-acting IL-15 receptor agonist was comparable to the response produced by IL-15.

### Example 8

### In Vitro Induction of Human NK Cell Proliferation Following Treatment with a Long-Acting IL-15 Receptor Agonist

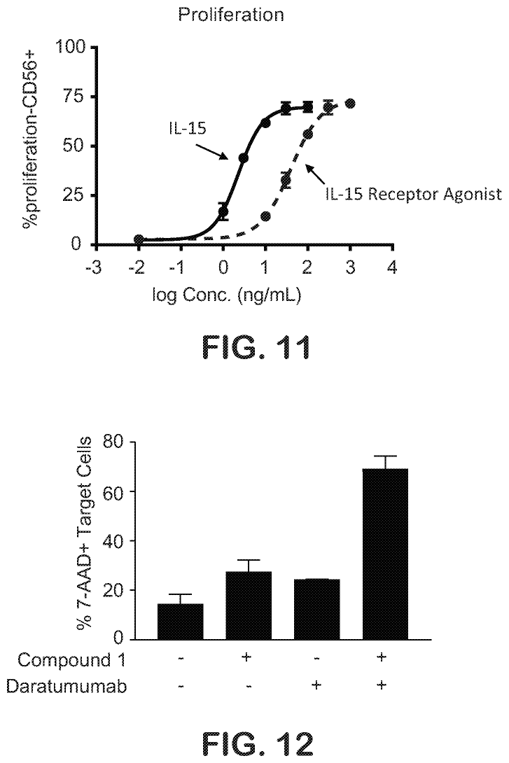

Human peripheral blood mononuclear cell (PBMCs) labeled with a CFSE preparation were incubated for 5 days in vitro in the presence of increasing doses of rhIL-15 (dose range 1 ng/ml-100 ng/ml, 5 point, 3 fold dilutions) or a long-acting IL-15 receptor agonist (mono(mPEG-butanamide)IL-15) (dose range 10 ng/ml-1000 ng/ml, 5 point, 3 fold dilutions). After the incubation period, cell proliferation as indicated by the decrease of CFSE in the cells that had undergone cell doublings was measured with flow cytometry measuring CFSE levels in CD56+ human NK cells within the PBMCs preparation. Results are provided in FIG. 11 and Table 4 below.

TABLE 4
Proliferation of human PBMCs in vitro.

| PBMCs treatment | EC50 (ng/ml)-% proliferation | Emax-% proliferation |
|---|---|---|
| rhIL-15 | 2.28 | 69.6% |
| long-acting IL-15 | 39.04 | 71.6% |
| receptor agonistα | | |

Both rhIL-15 and the long-acting IL-15 receptor agonist dose-dependently induced human NK cell proliferation. The long-acting IL-15 receptor agonist is approximately 17 fold less potent than rhIL-15 in inducing human NK cell proliferation. However, the maximum proliferation response produced by rhIL-15 and the long-acting IL-15 receptor agonist are comparable.

### Example 9

### Evaluation of NK Cell Cytotoxicity Following Treatment with a Long-Acting IL-15 Receptor Agonist and an Immunoglobulin

Human NK cells were magnetically sorted from normal donor PBMCs using negative selection. NK cells were seeded in a 96-well U-bottom plate at a density of 400,000 cells/well. NK cells were stimulated with 1000 ng/mL of a long-acting IL-15 receptor agonist (mono(mPEG-butanamide)IL-15) (+) or left unstimulated (−) in complete RPMI-1640 media supplemented with 10% fetal bovine serum (FBS) and 1% antibiotic/antimycotic overnight at 37° C. in 5% CO₂.

The human multiple myeloma RPMI-8226 cells (ATCC #CCL-155) were washed in PBS and stained with 0.15 μM CFSE for 5 min at 37° C. The tumor cells were pre-coated with 100 ng/mL daratumumab (+) for 1 hr at 37° C. where indicated. The stimulated NK cells (effector cells) and the pre-coated CFSE-labeled tumor cells (target cells) were co-cultured in an E:T ratio of 10:1 for 3 hr at 37° C. in 5% CO₂. Following incubation, cells were washed in flow staining buffer, Fc-blocked for 15 min at 4° C., and surface-stained (CD45, CD56, CD3-fluorescently conjugated antibodies). The cells were washed in PBS and incubated in 7-aminoactinomycin (7-AAD) viability stain for 30 minutes at 4° C. before acquiring for flow cytometric analysis using a Fortessa (BD). The percentage of 7-AAD+CFSE+ target cells from total cell population is the percentage for dead tumor cells.

Results are provided in FIG. 12. The data shows that the long-acting IL-15 receptor agonist-primed human NK cells enhanced ADCC of daratumumab pre-coated multiple myeloma cells. NK cells primed with a long-acting IL-15 receptor agonist were more cytotoxic to target cells than untreated NK cells or NK cells treated with daratumumab alone.

### Example 10

### NK Cell Low Affinity Immunoglobulin Receptor (CD16) Cell Surface Expression Modulation Following Treatment with Tumor Targeting Therapeutic Antibodies and a Long-Acting IL-15 Receptor Agonist

Balb/c mice bearing Daudi B cell lymphoma tumors in the bone marrow (n=6/group) were intraperitoneally administered a single 0.5 mg/kg dose of the human CD38 protein targeting clinical antibody daratumumab (DARZALEX®, Janssen Biotech) or a nonspecific control antibody with a matching isotype on day 14 after Daudi cell intravenous inoculation. Additional groups were treated with (i) mono(mPEG-N-butanamide)_{40 kD} interleukin-15 (Conjugate 1) administered subcutaneously at a dose of 0.3 mg/kg as a single agent on days 14 and 21 after Daudi cell inoculation, or (ii) a combination treatment of Conjugate 1 administered subcutaneously at doses of 0.03 mg/kg or 0.3 mg/kg on days 14 and 21 after Daudi cell inoculation and daratumumab administered intraperitoneally at a dose of 0.5 mg/kg on day 14 after Daudi cell inoculation.

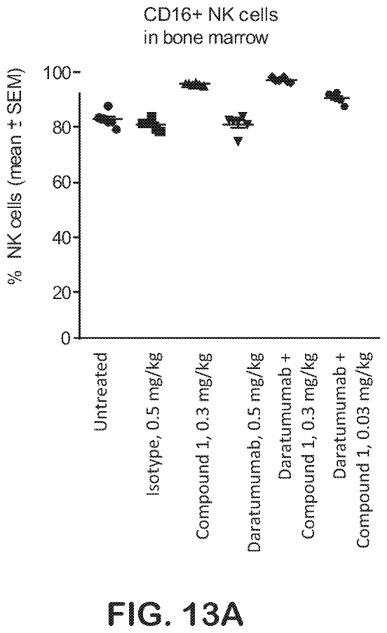

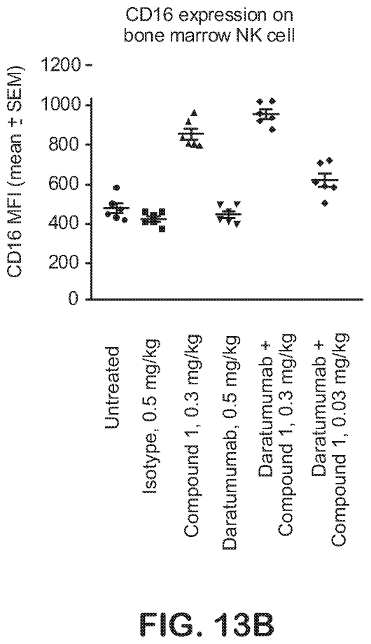

Three days following the second Conjugate 1 administration (day 24 after Daudi inoculation), bone marrow was collected and the NK cell fraction was measured by flow cytometry in total bone marrow. The fraction of NK cells expressing the cell surface CD16 receptor that is known to mediate antibody dependent cellular cytotoxicity mechanism in the presence of tumor targeting antibodies, and the change in CD16 expression per cell basis on NK cells are shown in FIGS. 13A and 13B, respectively.

Results are provided in Table 5 and Table 6 and FIGS. 13A-13B. All groups that received a therapeutic dose of Conjugate 1 as a single agent or in combination with daratumumab showed an increased fraction of NK cells that expressed CD16 on the cell surface (FIG. 13A) and CD16 expression was also increased on NK cells on a per cell basis (FIG. 13B) when compared to all of the treatments not including Conjugate 1. The respective increase in CD16 receptor expressing NK cells as well as the increase of CD16 expression on individual NK cells was indicative of NK cell activation by Conjugate 1 in the presence of therapeutic tumor targeting antibody as exemplified by daratumumab in this example.

TABLE 5
Fraction of NK cells expressing CD16 on cell surface

| | Fraction of NK cell |
|---|---|
| | expressing cell surface |
| Treatment | CD16 (mean value) |
| Untreated | 83% |
| Isotype control antibody | 81% |
| Conjugate 1, 0.3 mg/kg | 96% |
| Daratumumab, 0.5 mg/kg | 81% |
| Daratumumab + Conjugate 1, 0.3 mg/kg | 97% |
| Daratumumab + Conjugate 1, 0.03 mg/kg | 91% |

TABLE 6
Per cell basis CD16 expression on NK cells

| | CD16 mean | |
|---|---|---|
| | fluorescence | Relative CD16 |
| | intensity (MFI | expression compared |
| Treatment | mean value) | to untreated condition |
| Untreated | 477 | NA |
| Isotype control antibody | 425 | No increase (0.9x) |
| Conjugate 1, 0.3 mg/kg | 853 | 1.8x increase |
| Daratumumab, 0.5 mg/kg | 448 | No increase (1x) |
| Daratumumab + Conjugate 1, | 954 | 2x increase |
| 0.3 mg/kg | | |
| Daratumumab + Conjugate 1, | 663 | 1.4x increase |
| 0.03 mg/kg | | |

### Example 11

### NK Cell Cytotoxic Protein Granzyme B Expression Modulation Following Treatment with Tumor Targeting Therapeutic Antibodies and a Long-Acting IL-15 Receptor Agonist

Balb/c mice bearing Daudi B cell lymphoma tumors in the bone marrow (n=6/group) were intraperitoneally administered a single 0.5 mg/kg dose of the human CD38 protein targeting clinical antibody daratumumab (DARZALEX®), Janssen Biotech) or a nonspecific control antibody with a matching isotype on day 14 after Daudi cell intravenous inoculation. Additional groups were treated with (i) mono(mPEG-N-butanamide)_{40 kD} interleukin-15 (Conjugate 1) administered subcutaneously at a dose of 0.3 mg/kg as a single agent on days 14 and 21 after Daudi cell inoculation, or (ii) a combination treatment of Conjugate 1 administered subcutaneously at doses of 0.03 mg/kg or 0.3 mg/kg on days 14 and 21 after Daudi cell inoculation and daratumumab administered intraperitoneally at a dose of 0.5 mg/kg on day 14 after Daudi cell inoculation.

Three days following the second Conjugate 1 administration (day 24 after Daudi inoculation), bone marrow was collected and the NK cell fraction was measured by flow cytometry in total bone marrow. The change of intracellular Granzyme B expression on a per cell basis on NK cells is shown in FIG. 14.

Results are provided in Table 7 and FIG. 14. Groups that received a therapeutic dose of Conjugate 1 as a single agent or in combination with daratumumab showed increased expression of Granzyme B on a per cell basis in NK cells (FIG. 14) when compared to all of the treatments not including Conjugate 1. The respective increase in Granzyme B expression in individual NK cells was indicative of NK cell activation by Conjugate 1 in the presence of a therapeutic tumor targeting antibody as exemplified by daratumumab in this example.

TABLE 7
Per cell basis Granzyme B expression in NK cells

| | | Relative |
|---|---|---|
| | | Granzyme B |
| | Granzyme B mean | expression |
| | fluorescence | compared to |
| | intensity (MFI | untreated |
| Treatment | mean value) | condition |
| Untreated | 396 | NA |
| Isotype control antibody | 312 | No increase (0.8x) |
| Conjugate 1, 0.3 mg/kg | 2649 | 6.7x increase |
| Daratumumab, 0.5 mg/kg | 272 | No increase (0.7x) |
| Daratumumab + Conjugate 1, | 2056 | 5.2x increase |
| 0.3 mg/kg | | |
| Daratumumab + Conjugate 1, | 431 | No increase (1x) |
| 0.03 mg/kg | | |

### Example 12

### Evaluation of Antibody-Mediated Antibody-Dependent Cellular Cytotoxicity (ADCC) on B Cell Lymphoma Cells in Vitro Following Treatment with a Long-Acting IL-15 Receptor Agonist

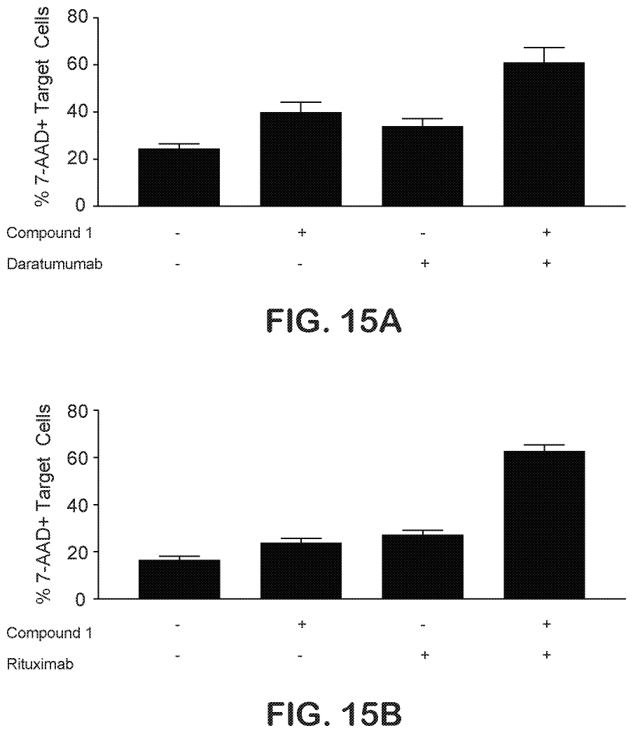

Human NK cells were magnetically sorted from normal donor PBMCs using negative selection. NK cells were seeded in a 96-well U-bottom plate at a density of 80,000 cells/well (FIG. 15A) OR 400,000 cells/well (FIG. 15B). NK cells were stimulated with 300 ng/mL of a long-acting IL-15 receptor agonist (mono(mPEG-butanamide)IL-15) (+) or left unstimulated (−) in complete RPMI-1640 media supplemented with 10% fetal bovine serum (FBS) and 1% antibiotic/antimycotic overnight at 37° C. in 5% CO₂.

The human multiple myeloma RPMI-8226 cells (ATCC #CCL-155) were washed in PBS and stained with 0.15 μM CFSE for 5 min at 37° C. The stimulated NK cells (effector cells) and the daratumumab-coated (+1 μg/mL) CFSE-labeled tumor cells (target cells) were concurrently co-cultured in an E:T ratio of 10:1 for 3 hours at 37° C. in 5% CO₂. Following incubation, cells were washed in flow staining buffer, Fc-blocked for 15 min at 4° C., and surface-stained (CD45, CD56, CD3-fluorescently conjugated antibodies). The cells were washed in PBS and incubated in 7-aminoactinomycin (7-AAD) viability stain for 30 minutes at 4° C. before acquiring for flow cytometric analysis using a Fortessa (BD). The percentage of 7-AAD+ CFSE+ target cells from total cell population is the percentage for dead tumor cells. Results are provided in FIG. 15A.

Alternatively, the human B cell lymphoma (Daudi) cells were washed in PBS and stained with 0.3 μM CFSE for 5 min at 37° C. The rituximab-coated (+10 ng/mL) CFSE-labeled tumor (target cells) cells were concurrently co-cultured with the NK cells (effector cells) in an E:T ratio of 2:1 for 3 hr at 37° C. in 5% CO₂. Following incubation, cells were washed in flow staining buffer, Fc-blocked for 15 min at 4° C., and surface-stained (CD45, CD56, CD3-fluorescently conjugated antibodies). The cells were washed in PBS and incubated in 7-aminoactinomycin (7-AAD) viability stain for 30 minutes at 4° C. before acquiring for flow cytometric analysis using a Fortessa (BD). The percentage of 7-AAD+ CFSE+ target cells from total cell population is the percentage for dead tumor cells. Results are provided in FIG. 15B.

The data shows that long-acting IL-15 receptor agonist-primed human NK cells enhance ADCC of daratumumab-coated and rituximab-coated B cell lymphoma cells. long-acting IL-15 receptor agonist-primed NK cells are more cytotoxic to target cells than untreated NK cells.

### Example 13

### Evaluation of Survival in Daudi B Cell Lymphoma Xenograft Models Following Administration of a Long-Acting IL-15 Receptor Agonist and an Anti-CD38 Monoclonal Antibody, Daratumumab

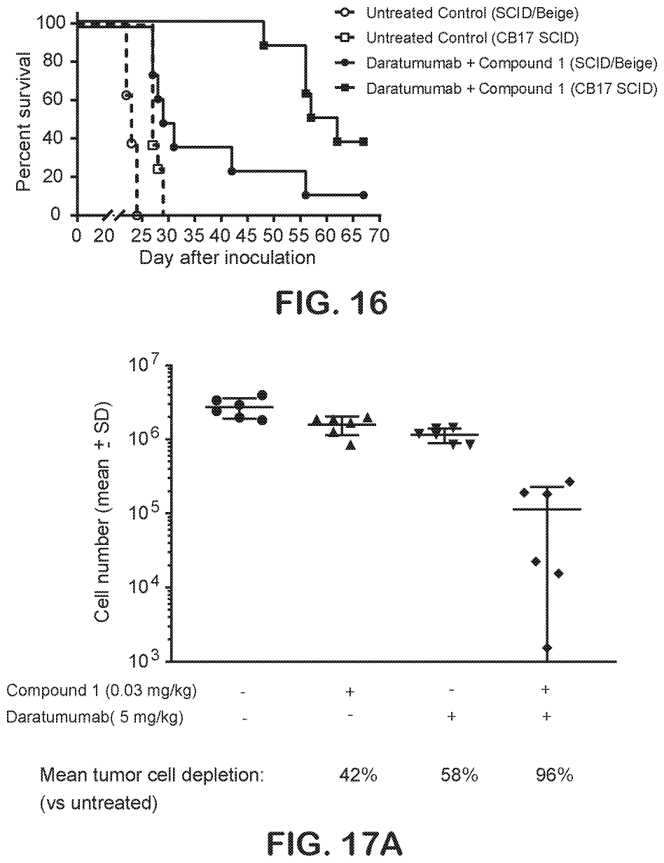

SCID or SCID beige mice (N=8/group) inoculated intravenously with 10 million Daudi B cell lymphoma cells were treated with a single dose of daratumumab (0.5 mg/kg, IP, 14 days after inoculation) and a total of three doses of mono(methoxyPEG-N-butanamide)_{40 kD} interleukin-15 (compound 1) (0.3 mg/kg, intravenously, administered 14, 21 and 28 days after tumor inoculation). Also evaluated were untreated SCID/beige or CB17 SCID mice as a control. Survival of tumor-inoculated mice was measured by body condition scoring and hind limb paralysis onset as endpoint markers. Results are provided graphically in FIG. 16.

As shown in FIG. 16, the mono(methoxyPEG-N-butanamide)interleukin-15 combination with daratumumab treatment led to a more than double increase of the survival rate (33 days) from median survival of 27 days in untreated control SCID mice to 60 days in combination treated mice. In contrast the mono(methoxyPEG-N-butanamide)interleukin-15 combination with daratumumab treatment in SCID mice harboring the beige mutation that reduces NK cells functionality (SCID beige) led to only an increase of survival rate by 7 days (from 23 days in untreated SCID beige mice to 30 days in combination treated SCID beige mice). These results indicate that NK cell activity is required for the full therapeutic effect of the mono(methoxyPEG-N-butanamide)interleukin-15 combination with daratumumab treatment.

### Example 14

### Evaluation of Cell Counts in a Daudi B Cell Lymphoma Xenograft Model Following Administration of a Long-Acting IL-15 Receptor Agonist and an Exemplary Anti-CD38 Monoclonal Antibody, Daratumumab

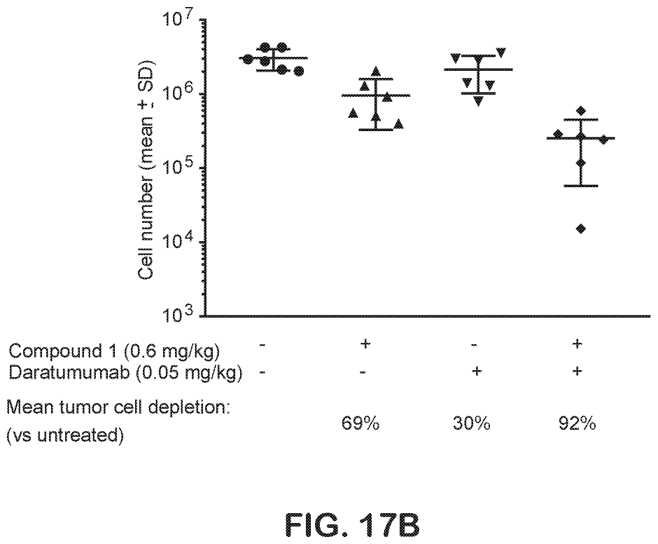

SCID mice (N=6/group) were intravenously inoculated with 10 million Daudi cells on Day 0 and treated with a single dose of a lower or higher dose of daratumumab (0.05 mg/kg or 5 mg/kg IP, 14 days following Daudi cell inoculation) and two doses of either lower or higher dose mono(methoxyPEG-N-butanamide)interleukin-15 (compound 1) (0.03 mg/kg or 0.6 mg/kg intravenously, one dose at each of 14 and 21 days after inoculation). Mice were also treated with the single agent therapies: daratumumab (0.05 mg/kg or 5 mg/kg IP, 14 days following Daudi cell inoculation) or two doses of mono(methoxyPEG-N-butanamide)_{40 kD} interleukin-15 (compound 1) (0.03 mg/kg or 0.6 mg/kg intravenously, 14 and 21 days after inoculation). Daudi cell numbers in the bone marrow were assessed three days following the second dose of mono(methoxyPEG-N-butanamide)_{40 kD} interleukin-15 (day 24) by flow cytometry as shown in FIGS. 17A and 17B.

As shown in FIGS. 17A-17B, the mono(methoxyPEG-N-butanamide)interleukin-15 (compound 1) combination with daratumumab depleted intra-bone marrow tumor cells with comparable efficacy when high daratumumab dose level (5 mg/kg) was combined with low dose mono(methoxyPEG-N-butanamide)interleukin-15 (0.03 mg/kg) (FIG. 17A), depleting 96% of the tumor cells, compared to when low dose daratumumab (0.05 mg/kg) was combined with high dose mono(methoxyPEG-N-butanamide)interleukin-15 (0.6 mg/kg) (FIG. 17B) depleting 92% of the tumors cells. As single agent treatments, low dose daratumumab and mono(methoxyPEG-N-butanamide)interleukin-15 depleted only 30% and 42% of the tumor cells, respectively. As single agent treatments, high dose daratumumab and mono(methoxyPEG-N-butanamide)interleukin-15 depleted only 58% and 69% of the tumor cells, respectively. These results indicate that reduction in dose level or tissue concentration in daratumumab treatment can be compensated by an increase in the mono(methoxyPEG-N-butanamide)interleukin-15 dose level or tissue concentration to maintain high efficacy of tumor cell killing by the combination treatment.

### Example 15

### Evaluation of an HCT-116 Colorectal Carcinoma Cell Model Following Administration of a Long-Acting IL-15 Receptor Agonist and an Exemplary Anti-EGFR Monoclonal Antibody, Cetuximab

HCT-116 cells were cultured in RPMI1640 with 5% FBS for two weeks prior to inoculation. Balb/c SCID mice (N=4/group) were subcutaneously inoculated with 5 million cells HCT-116 cells. 7 days after tumor cell inoculation at mean tumor volume ˜150 mm³ (Day 0) mice were treated with a single dose of the anti-human EGFR antibody, cetuximab (20 mg/kg IP) and a single dose of mono(methoxyPEG-N-butanamide)interleukin-15 (Compound 1) (0.3 mg/kg IV).

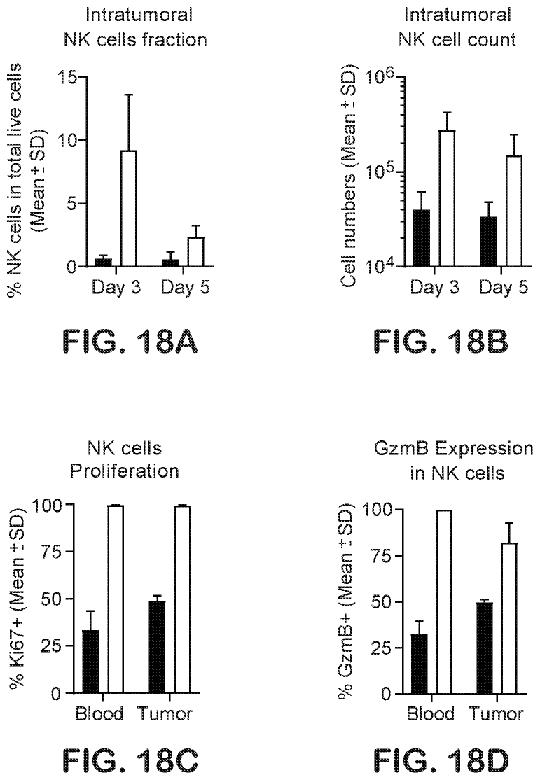

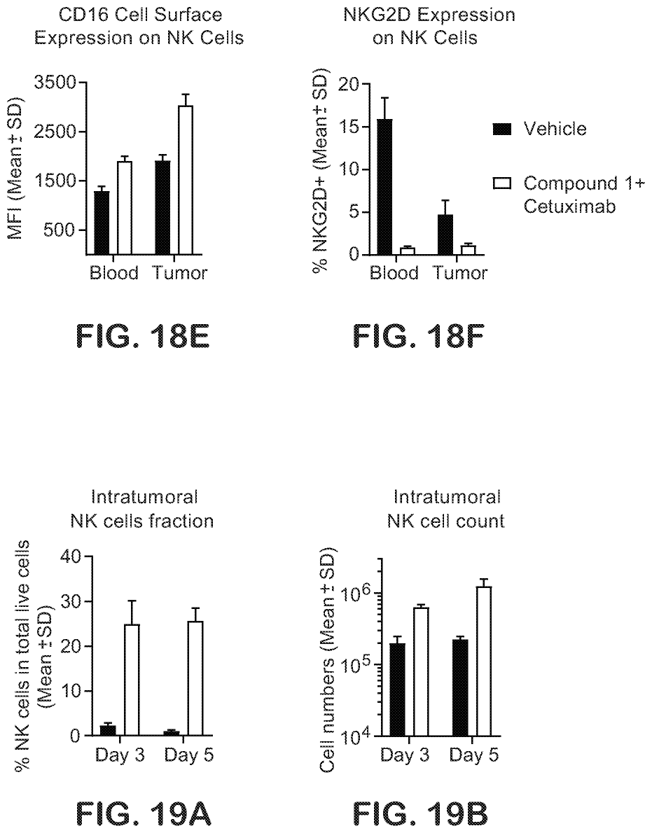

Three and five days following treatment, blood and tumor tissue were collected and the NK cell fraction was measured by flow cytometry as shown in FIG. 18A. As seen in FIG. 18A, an increase in NK cell fraction within total live cells in the tumors was induced by the treatment. Three and five days following treatment, blood and tumor tissue were collected and the NK cell numbers were measured by flow cytometry as shown in FIG. 18B. As shown in FIG. 18B, an increase in the NK cell numbers in the tumor tissue was induced by the treatment.

Samples were subjected to immunophenotyping for % Ki-67 by flow cytometry (see FIG. 18C) at day 3 following treatment. FIG. 18C is a plot of NK cell proliferation, measured by % Ki-67 positivity, used as a marker for proliferating cells, over time. *Ki67 marker induction was observed in a majority of the NK cells indicating effective proliferative response after treatment.

Granzyme B expression was measured in blood or tumor NK cells on day 3 by flow cytometry after treatment as shown in FIG. 18D. As illustrated therein, an increase in GzmB expression was observed in both blood and tumor NK cells after treatment indicating cytotoxic activation of the NK cells by the treatment.

The fraction of blood or tumor NK cells expressing the cell surface CD16 receptor was measured with flow cytometry by measuring the increase of median fluorescence intensity (MFI) signal of CD16 as shown in FIG. 18E. As illustrated therein, an increase in cell surface CD16 expression was induced by the treatment indicating NK cell activation.

Three days following treatment, blood and tumor cells were collected and expression of the cell surface activating receptor NKG2D were measured by flow cytometry with the results shown in FIG. 18F. As illustrated therein, NKG2D surface expression was reduced consistent with functional activation of NK cells by the treatment.

These results further suggest that mono(methoxyPEG-N-butanamide)interleukin-15 is effective to substantially enhance NK-cell mediated ADCC when administered with an anti-tumor antibody.

### Example 16

### Evaluation of a FaDu Human Head and Neck Squamous Cell Carcinoma Model Following Administration of a Long-Acting IL-15 Receptor Agonist and an Exemplary Anti-EGFR Monoclonal Antibody, Cetuximab

Balb/c SCID mice (N=4/group) were intravenously inoculated with 3 million FaDu cells. 7 days after tumor cell inoculation at mean tumor volume ˜150 mm³ (Day 0) mice were treated with a single dose of the anti-human EGFR antibody, cetuximab, (20 mg/kg IP) and a single dose of mono(methoxyPEG-N-butanamide)interleukin-15 (Compound 1) (0.3 mg/kg IV) ˜150 mm³.

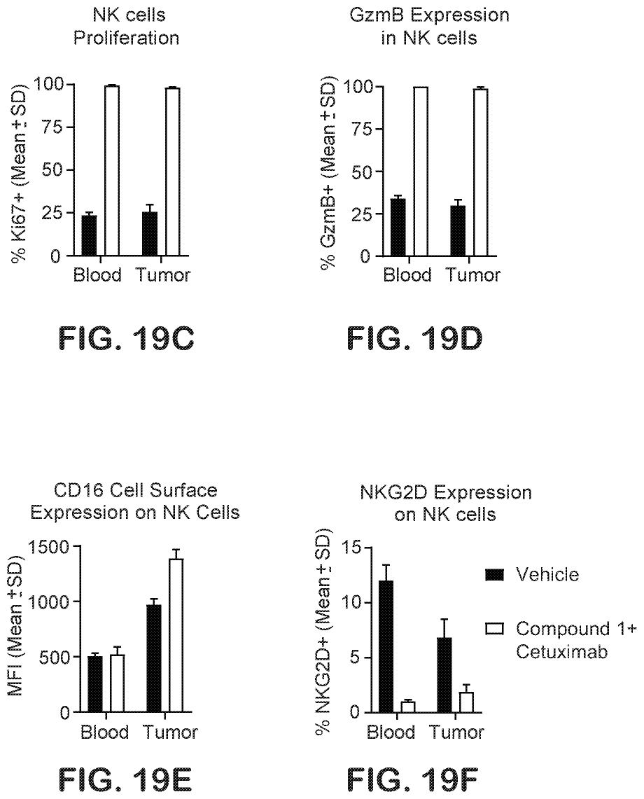

Three and five days following treatment, blood and tumor tissue were collected and the NK cell fraction was measured by flow cytometry is shown in FIG. 19A. As shown in FIG. 19A, an increase in the NK cell fraction within total live cells in the tumors was induced by the treatment. Three and five days following treatment, blood and tumor tissue were collected and the NK cell numbers were measured by flow cytometry as shown in FIG. 19B. As shown in FIG. 19B, an increase in the NK cell numbers in the tumor tissue was induced by the treatment.

Samples were subjected to immunophenotyping for % Ki-67 (see FIG. 19C) at day 3 following treatment. FIG. 19C is a plot of NK cell proliferation, measured by % Ki-67 positivity, used as a marker for proliferating cells, over time. Ki67 marker induction was observed in a majority of the NK cells indicating effective proliferative response after treatment.

Granzyme B expression was measured in blood or tumor NK cells on day 3 by flow cytometry after treatment as shown in FIG. 19D. As illustrated therein, an increase in GzmB expression was observed both in blood and tumor NK cells after treatment indicating cytotoxic activation of NK cells by the treatment.

The fraction of blood or tumor NK cells expressing the cell surface CD16 receptor was measured with flow cytometry by measuring the increase of median fluorescence intensity (MFI) signal of CD16 as shown in FIG. 19E. As illustrated therein, an increase in cell surface CD16 expression was induced by the treatment indicating NK cell activation.

Three days following treatment, blood and tumor cells were collected and expression of the cell surface activating receptor NKG2D were measured by flow cytometry with the results shown in FIG. 19F. As illustrated therein, NKG2D surface expression was reduced consistent with functional activation of NK cells by the treatment.

These results further suggest that mono(methoxyPEG-N-butanamide)interleukin-15 is effective to substantially enhance NK-cell mediated ADCC when administered with an anti-tumor antibody.

### Example 17

### Evaluation of Tumor Growth Inhibition in a H1975 Lung Tumor Model Following Administration of a Long-Acting IL-15 Receptor Agonist and an Exemplary Anti-EGFR Monoclonal Antibody, Cetuximab

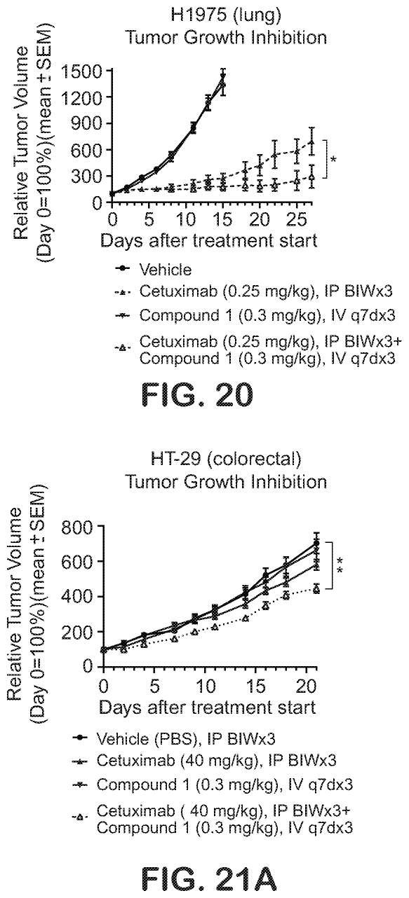

Balb/c nude mice (N=10/group) inoculated subcutaneously on flank with 5 million H1975 cells were treated with total of 3 doses of cetuximab (0.25 mg/kg, IP, administered 9, 12 and 16 days after inoculation) and a total of three doses of mono(methoxyPEG-N-butanamide)_{40 kD} interleukin-15 (Compound 1) (0.3 mg/kg, intravenous), administered 9, 16 and 23 days after tumor inoculation. Delay in loss of tumor growth control was evaluated on day 27 after treatment initiation. Results are provided graphically in FIG. 20.

As shown in FIG. 20, the mono(methoxyPEG-N-butanamide)interleukin-15 treatment led to no tumor growth inhibition in treated mice. Cetuximab treated mice showed resumption of tumor growth after cetuximab treatment schedule had been completed. In contrast the mono(methoxyPEG-N-butanamide)interleukin-15 combination with cetuximab treatment led to a delay in tumor growth relapse by day 27 after treatment initiation showing significantly slower tumor growth (p=0.02, Mann-Whitney test) in the mono(methoxyPEG-N-butanamide)interleukin-15 combination with cetuximab treatment compared to cetuximab single agent treatment. These data indicate that the mono(methoxyPEG-N-butanamide)interleukin-15 combination with cetuximab leads to superior tumor growth control compared to the cetuximab single agent treatment in this tumor model.

### Example 18

### Evaluation of Tumor Growth Inhibition and Delay in a HT-29 Colorectal Carcinoma Cell Model Following Administration of a Long-Acting IL-15 Receptor Agonist and an Exemplary Anti-EGFR Monoclonal Antibody, Cetuximab

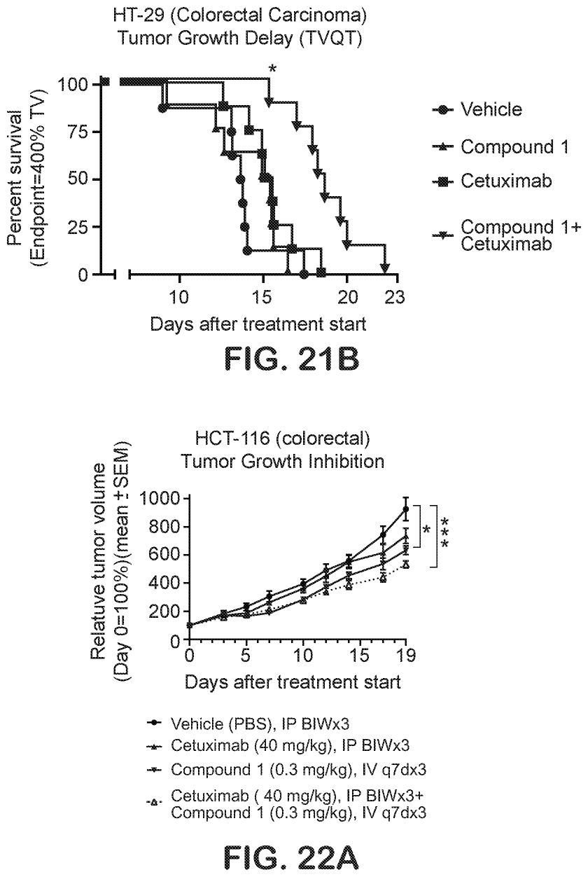

SCID mice (N=8/group) inoculated subcutaneously on flank with 5 million HT-29 cells were treated with total of six doses of cetuximab (40 mg/kg, IP, administered 7, 11, 14, 18, 21, 25 days after inoculation) and a total of three doses of mono(methoxyPEG-N-butanamide)_{40 kD} interleukin-15 (Compound 1) (0.3 mg/kg, intravenous), administered 7, 14 and 21 days after tumor inoculation. Tumor growth inhibition was evaluated on day 21 after treatment initiation. Results are provided graphically in FIG. 21A.

As shown in FIG. 21A, the mono(methoxyPEG-N-butanamide)interleukin-15 or cetuximab single agent treatments led to no tumor growth inhibition in treated mice. In contrast the mono(methoxyPEG-N-butanamide)interleukin-15 combination with cetuximab treatment led to significant tumor growth inhibition. These data indicate that the mono(methoxyPEG-N-butanamide)interleukin-15 combination with cetuximab leads to tumor growth inhibition in a cetuximab resistant tumor model.

The tumor growth delay (TVQT) was evaluated with the endpoint set as 400% tumor growth from baseline. FIG. 21B shows that 38% tumor growth delay was induced by measuring tumor volume quadrupling time (TVQT) from baseline tumor volume on treatment initiation day.

### Example 19

### Evaluation of Tumor Growth Inhibition and Delay in HCT-116 Colorectal Carcinoma Xenograft Model Following Administration of a Long-Acting IL-15 Receptor Agonist and an Exemplary Anti-EGFR Monoclonal Antibody, Cetuximab

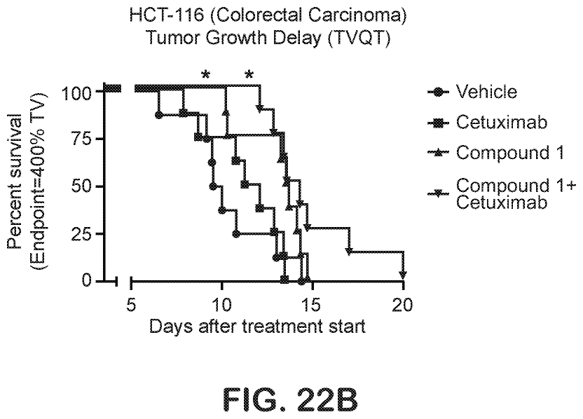

SCID mice (N=8/group) inoculated subcutaneously on flank with 5 million HCT-116 cells were treated with total of six doses of cetuximab (40 mg/kg, IP, administered 7, 11, 14, 18, 21, 25 days after inoculation) and a total of three doses of mono(methoxyPEG-N-butanamide)_{40 kD} interleukin-15 (Compound 1) (0.3 mg/kg, intravenously, administered 7, 14 and 21 days after tumor inoculation). Tumor growth inhibition was evaluated on day 19 after treatment initiation. Results are provided graphically in FIG. 22A.

As shown in FIG. 22A, cetuximab single agent treatment led to no tumor growth inhibition in treated mice. The mono(methoxyPEG-N-butanamide)interleukin-15 single agent treatment led to 31% tumor growth inhibition. In contrast the mono(methoxyPEG-N-butanamide)interleukin-15 combination with cetuximab treatment led to 42% tumor growth inhibition. These data indicate that the mono(methoxyPEG-N-butanamide)interleukin-15 combination with cetuximab leads to tumor growth inhibition in a cetuximab resistant tumor model.

The tumor growth delay was evaluated by measuring tumor volume quadrupling time (TVQT) from baseline tumor volume on treatment initiation day. FIG. 22B shows that 42% tumor growth delay was induced by the combination treatment and 26% tumor growth delay was induced by mono(methoxyPEG-N-butanamide)_{40 kD} interleukin-15 (Compound 1) single agent treatment while cetuximab single agent treatment produced no tumor growth delay.

### Example 20

### Evaluation of Antibody-Mediated Cellular Cytotoxicity (ADCC) in HCT-116 Colorectal Carcinoma In Vitro Model Following Administration of a Long-Acting IL-15 Receptor Agonist and an Exemplary Anti-EGFR Monoclonal Antibody, Cetuximab

Human NK cells were magnetically sorted from normal donor PBMCs using negative selection. NK cells were seeded in a 96-well U-bottom plate at a density of 400,000 cells/well. NK cells were stimulated with mono(methoxyPEG-N-butanamide)_{40 kD} interleukin-15 (Compound 1) (300 ng/mL) (+) or left unstimulated (−) in complete RPMI-1640 media supplemented with 10% fetal bovine serum (FBS) and 1% antibiotic/antimycotic overnight at 37° C. in 5% CO₂.

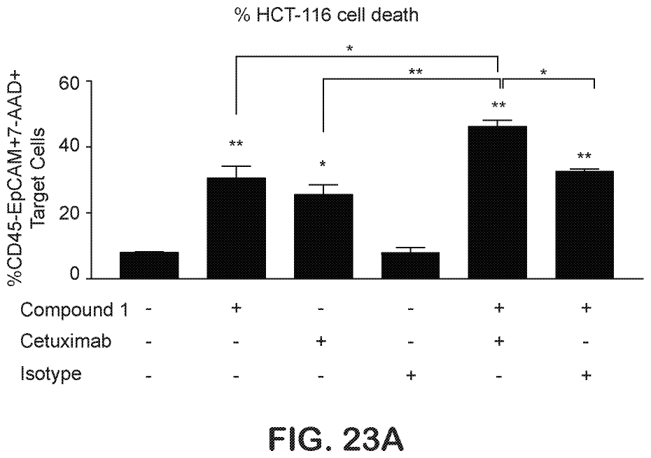

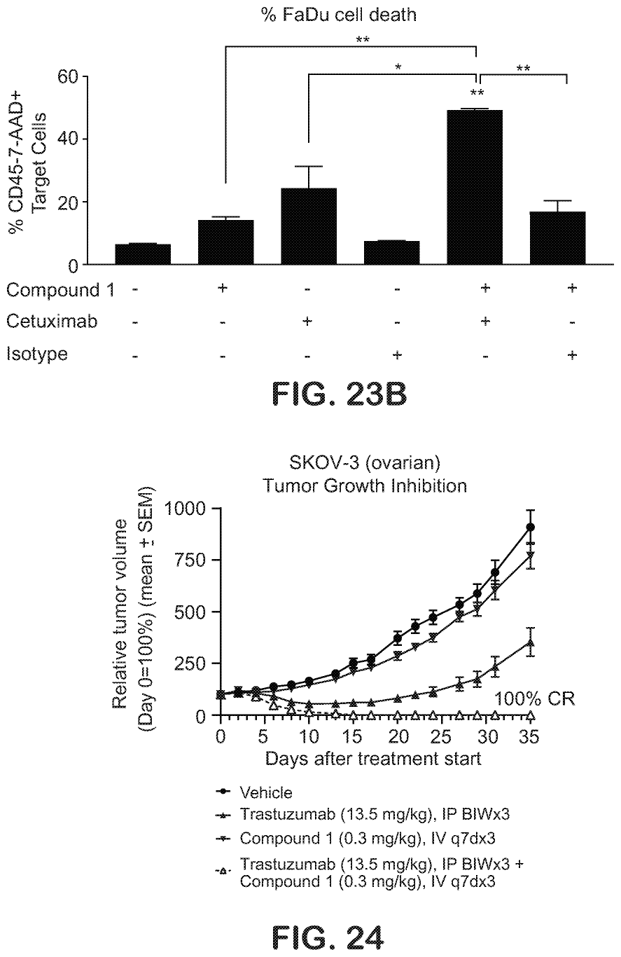

The colonic human HCT-116 cells (ATCC #CCL-247) were cultured McCoy 5A media (ATCC+30-2007) supplemented with 10% fetal bovine serum (FBS) and 1% antibiotic/antimycotic at 37° C. in 5% CO₂. HCT-116 cells were subcultured by incubating in 0.25% Trypsin-0.53 mM EDTA for 5 min at 37° C., washed in sterile PBS, resuspended in culture media, counted, and co-cultured with the NK cells using an effector:target (E:T) of 10:1. Concurrently, Cetuximab (McKesson Medical Surgical #66733-948-23 lot #C1800115) and an IgG1 isotype (Biolegend; LEAF Purified IgG1) were added (+) to the corresponding wells at a concentration of 30 ug/mL before incubating for 3 hours at 37° C. in 5% CO₂. Following incubation, cells were washed in flow staining buffer, Fc-blocked for 15 min at 4° C., and surface-stained (EpCAM, CD45, CD56, CD3-fluorescently conjugated antibodies) for 20 min at 4° C. in the dark. The cells were washed in PBS and incubated in 7-aminoactinomycin (7-AAD) viability stain for 30 minutes at 4° C. before acquiring for flow cytometric analysis using a Fortessa (BD). The percentage of 7-AAD+CD45-EpCAM+ target cells from total cell population is the percentage for dead tumor cells. Results are provided in FIG. 23A.

The epithelial/pharyngeal head and neck human FaDu cells (ATCC #HTB-43) were cultured in minimal eagle's essential media (EMEM; ATCC+30-2003) supplemented with 10% fetal bovine serum (FBS) and 1% antibiotic/antimycotic at 37° C. in 5% CO₂. FaDu cells were subcultured by incubating in 0.25% Trypsin-0.53 mM EDTA for 5 min at 37° C., washed in sterile PBS, resuspended in culture media, counted, and co-cultured with the NK cells using an effector:target (E:T) of 10:1. Concurrently, Cetuximab (McKesson Medical Surgical #66733-948-23 lot #C1800115) and an IgG1 isotype (Biolegend; LEAF Purified IgG1) were added (+) to the corresponding wells at a concentration of 300 ng/mL before incubating for 3 hours at 37° C. in 5% CO₂. Following incubation, cells were washed in flow staining buffer, Fc-blocked for 15 min at 4° C., and surface-stained (CD45, CD56, CD3-fluorescently conjugated antibodies) for 20 min at 4° C. in the dark. The cells were washed in PBS and incubated in 7-aminoactinomycin (7-AAD) viability stain for 30 minutes at 4° C. before acquiring for flow cytometric analysis using a Fortessa (BD). The percentage of 7-AAD+CD45− target cells from total cell population is the percentage for dead tumor cells. Results are provided in FIG. 23B.

As seen in FIGS. 23A and 23B, the mono(methoxyPEG-N-butanamide)_{40 kD} interleukin-15-primed human NK cells enhance ADCC of cetuximab-coated human solid tumor cells.

### Example 21

### Evaluation of Tumor Growth Inhibition in a SKOV-3 Ovarian Carcinoma Cell Model Following Administration of a Long-Acting IL-15 Receptor Agonist and an Exemplary Anti-Her Monoclonal Antibody, Trastuzumab

Balb/c nude mice (N=10/group) inoculated subcutaneously on flank with 10 million SKOV-3 cells mixed with matrigel in 1:1 ratio were treated with total of six doses of trastuzumab (13.5 mg/kg, IV, administered twice a week for 3 weeks starting on day 6 after inoculation) and a total of three doses of mono(methoxyPEG-N-butanamide)_{40 kD} interleukin-15 (Compound 1) (0.3 mg/kg, intravenous), administered 6, 13 and 20 days after tumor inoculation). Tumor growth inhibition in inoculated mice was measured 35 days after treatment initiation. Results are provided graphically in FIG. 24.

As shown in FIG. 24, the mono(methoxyPEG-N-butanamide)interleukin-15 combination treatment led to no tumor growth inhibition in treated mice. Trastuzumab treated mice showed 61% tumor growth inhibition but no tumor free animals were produced by trastuzumab treatment. In contrast the mono(methoxyPEG-N-butanamide)interleukin-15 combination with trastuzumab treatment led to complete loss of tumors in all treated animals and the animals stayed tumor free. These data indicate that the mono(methoxyPEG-N-butanamide)interleukin-15 combination with trastuzumab leads to superior tumor growth inhibition compared to the trastuzumab single agent treatment in this tumor model.

### Example 22

### Evaluation of Tumor Growth Inhibition in an NCI-N87 Gastric Carcinoma Cell Model Following Administration of a Long-Acting IL-15 Receptor Agonist and an Exemplary Anti-Her Monoclonal Antibody, Trastuzumab

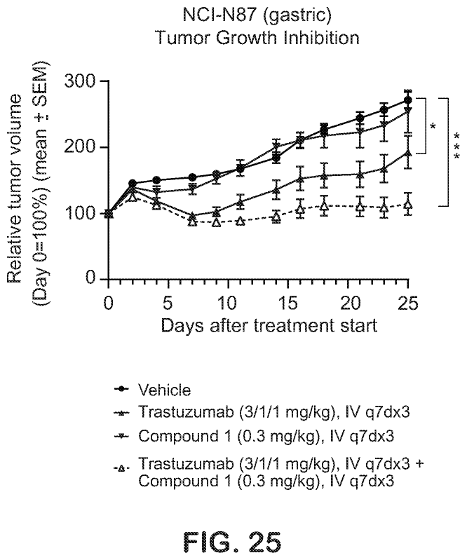

Balb/c nude mice (N=10/group) inoculated subcutaneously on flank with 10 million NCI-N87 cells mixed with matrigel in 1:1 ratio were treated with total of three doses of trastuzumab (first dose 3 mg/kg and subsequent two doses 1 mg/kg, IV, administered 5, 12 and 20 days after inoculation) and a total of three doses of mono(methoxyPEG-N-butanamide)_{40 kD} interleukin-15 (Compound 1) (0.3 mg/kg, intravenous), administered 5, 12 and 19 days after tumor inoculation. Tumor growth inhibition in inoculated mice was measured 25 days after treatment initiation. Results are provided graphically in FIG. 25.

As shown in FIG. 25, the mono(methoxyPEG-N-butanamide)interleukin-15 treatment led to no tumor growth inhibition in treated mice. Trastuzumab treated mice showed 29% tumor growth inhibition. In contrast the mono(methoxyPEG-N-butanamide)interleukin-15 combination with trastuzumab treatment led to higher 41% tumor growth inhibition in the treated mice. These data indicate that the mono(methoxyPEG-N-butanamide)interleukin-15 combination with trastuzumab leads to superior tumor growth inhibition compared to the trastuzumab single agent treatment in this tumor model.

Embodiments of the present methods, therapeutic combinations and kits include, but are not limited to:
Embodiment 1. A method of treating a subject having cancer, the method comprising: administering to the subject
   (a) a long-acting IL-15 receptor agonist having a structure:

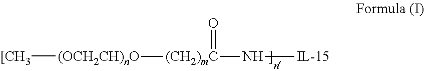

wherein IL-15 is an interleukin-15 moiety, n is an integer from about 150 to about 3,000, m is an integer selected from 2, 3, 4, and 5, n′ is 1, and ˜NH˜ represents an amino group of the IL-15 moiety; and
(b) a tumor-directed antibody that binds specifically to a tumor antigen selected from a phosphoprotein, a transmembrane protein, a glycoprotein, a glycolipid, and a growth factor, wherein the antibody has antibody dependent cellular cytotoxicity (ADCC) as a mechanism of action;
wherein steps (a) and (b) are carried out concurrently or sequentially and in any order.
Embodiment 2. The method of embodiment 1, wherein the long-acting IL-15 receptor agonist is a pharmaceutically acceptable salt.
Embodiment 3. The method of the combined or separate embodiments 1-2, wherein m in Formula (I) is 2 or 3.
Embodiment 4. The method of the combined or separate embodiments 1-3, wherein m in Formula (I) is 3.
Embodiment 5. The method of the combined or separate embodiments 1-4, wherein n in Formula (I) has a value of about 909.
Embodiment 6. The method of the combined or separate embodiments 1-5, wherein the long-acting IL-15 receptor agonist is selected from (methoxyPEG-N-butanamide)_{20-60 kD} interleukin-15, (methoxyPEG-N-butanamide)_{20-40 kD} interleukin-15, (methoxyPEG-N-butanamide)_{40 kD} interleukin-15, mono(methoxyPEG-N-butanamide)_{20-60 kD} interleukin-15, mono(methoxyPEG-N-butanamide)_{20-40 kD} interleukin-15, and mono(methoxyPEG-N-butanamide)_{40 kD} interleukin-15.
Embodiment 7. The method of the combined or separate embodiments 1-6, wherein the long-acting IL-15 receptor agonist is mono(methoxyPEG-N-butanamide)_{40 kD} interleukin-15. Embodiment 8. The method of the combined or separate embodiments 1-7, wherein the cancer is a solid cancer.
Embodiment 9. The method of the combined or separate embodiments 1-8, wherein the solid cancer is selected from the group consisting of breast cancer, ovarian cancer, colon cancer, colorectal cancer, gastric cancer, malignant melanoma, liver cancer, small cell lung cancer, non-small cell lung cancer, thyroid cancers, kidney cancer, cancer of the bile duct, brain cancer, cervical cancer, maxillary sinus cancer, bladder cancer, esophageal cancer, Hodgkin's disease and adrenocortical cancer, including metastatic forms of any of the foregoing.
Embodiment 10. The method of the combined or separate embodiments 1-7, wherein the cancer is a hematological malignancy.
Embodiment 11. The method of the combined or separate embodiments 1-7 and 10, wherein the hematological malignancy is selected from the group consisting of multiple myeloma, non-Hodgkin lymphoma, leukemia and lymphoma.
Embodiment 12. The method of the combined or separate embodiments 1-7 and 10-11, wherein the cancer is a B cell malignancy.
Embodiment 13. The method of the combined or separate embodiments 1-12, wherein step (a) is carried out prior to step (b).
Embodiment 14. The method of the combined or separate embodiments 1-12, wherein step (b) is carried out prior to step (a).
Embodiment 15. The method of the combined or separate embodiments 1-12, wherein step (a) and step (b) are carried out concurrently or substantially concurrently. Embodiment 16. The method of the combined or separate embodiments 1-15, wherein said
   administering is effective to stimulate NK activation to an extent greater than observed when either of the long-acting IL-15 receptor agonist or the tumor-directed antibody is administered as a single agent, as measured in a suitable animal model. Embodiment 17. The method of the combined or separate embodiments 1-16, wherein said
   administering is effective to stimulate NK proliferation to an extent greater than observed when either of the long-acting IL-15 receptor agonist or the tumor-directed antibody is administered as a single agent, as measured in a suitable animal model.
Embodiment 18. The method of the combined or separate embodiments 1-17, wherein said administering is effective to support CD8+ T-cell survival and memory formation to an extent greater than observed when either of the long-acting IL-15 receptor agonist or the tumor-directed antibody is administered as a single agent, as measured in a suitable animal model.
Embodiment 19. The method of the combined or separate embodiments 1-18, wherein the long-acting IL-15 receptor agonist is administered intravenously or subcutaneously. Embodiment 20. The method of the combined or separate embodiments, 1-19, wherein the long-acting IL-15 receptor agonist is administered q7d, q14d, q21d, or monthly, or any combination thereof.
Embodiment 21. The method of the combined or separate embodiments 1-20, wherein the tumor-directed antibody is administered intravenously or intraperitoneally. Embodiment 22. The method of the combined or separate embodiments, 1-21, wherein the tumor-directed antibody is administered q7d, q14d, q21d, or monthly, or any combination thereof.
Embodiment 23. The method of the combined or separate embodiments 1-22, wherein the tumor-directed antibody is a monoclonal antibody.
Embodiment 24. The method of the combined or separate embodiments 1-23, wherein the tumor-directed antibody is selected from an anti-CD19 antibody, an anti-CD20 antibody, and an anti-CD38 antibody.
Embodiment 25. The method of the combined or separate embodiments 1-23, wherein the tumor-directed antibody that binds specifically to a glycoprotein is selected from an anti-SLAMF7 antibody, an anti-EpCAM antibody, an anti-gpA3 antibody 3, and an anti-FBP antibody.
Embodiment 26. The method of the combined or separate embodiments 1-23, wherein the tumor-directed antibody that binds specifically to a growth factor is selected from an anti-VEGF antibody, an anti-VEGFR antibody, and an anti-EGFR antibody.
Embodiment 27. The method of the combined or separate embodiments 1-23, wherein the tumor-directed antibody is an IgG antibody.
Embodiment 28. The method of the combined or separate embodiments 1-23, wherein the tumor-directed antibody is selected from the group consisting of daratumumab, rituximab, cetuximab and trastuzumab.
Embodiment 29. A therapeutic combination for use in treating cancer, comprising
   (a) a long-acting IL-15 receptor agonist having a structure:

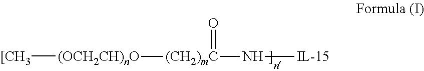

wherein IL-15 is an interleukin-15 moiety, n is an integer from about 150 to about 3,000, m is an integer selected from 2, 3, 4, and 5, n′ is 1, and ˜NH˜ represents an amino group of the IL-15 moiety; and
(b) a tumor-directed antibody that binds specifically to a tumor antigen selected from a phosphoprotein, a transmembrane protein, a glycoprotein, a glycolipid, and a growth factor, wherein the tumor-directed antibody includes antibody dependent cellular cytotoxicity (ADCC) as a mechanism of action.
Embodiment 30. The therapeutic combination of embodiment 29, wherein the long-acting receptor agonist is a pharmaceutically acceptable salt.
Embodiment 31. The therapeutic combination of the combined or separate embodiments 29-30, wherein m in Formula (I) is 2 or 3.
Embodiment 32. The therapeutic combination of the combined or separate embodiments 29-31, wherein m in Formula (I) is 3.
Embodiment 33. The therapeutic combination of the combined or separate embodiments 29-32, wherein n in Formula (I) has a value of about 909.
Embodiment 34. The therapeutic combination of the combined or separate embodiments 29-33, wherein the long-acting IL-15 receptor agonist is selected from (methoxyPEG-N-butanamide)_{20-60 kD} interleukin-15, (methoxyPEG-N-butanamide)_{20-40 kD} interleukin-15, (methoxyPEG-N-butanamide)_{40 kD} interleukin-15, mono(methoxyPEG-N-butanamide)_{20-60 kD} interleukin-15, mono(methoxyPEG-N-butanamide)_{20-40 kD} interleukin-15, and mono(methoxyPEG-N-butanamide)_{40 kD} interleukin-15.
Embodiment 35. The therapeutic combination of the combined or separate embodiments 29-34, wherein the long-acting IL-15 receptor agonist is mono(methoxyPEG-N-butanamide)_{40 kD} interleukin-15.
Embodiment 36. The therapeutic combination of the combined or separate embodiments 29-35, wherein the long-acting IL-15 receptor agonist is formulated for intravenous or subcutaneous administration.
Embodiment 37. The therapeutic combination of the combined or separate embodiments 29-36, wherein the tumor-directed antibody is formulated for intravenous or intraperitoneal administration.
Embodiment 38. The therapeutic combination of the combined or separate embodiments 29-37, wherein the long-acting IL-15 receptor agonist is formulated for intravenous or subcutaneous administration.
Embodiment 39. The therapeutic combination of the combined or separate embodiments 29-38, wherein the tumor-directed antibody is formulated for intravenous or intraperitoneal administration.
Embodiment 40. The therapeutic combination of the combined or separate embodiments 29-39, wherein the tumor-directed antibody is a monoclonal antibody.
Embodiment 41. The therapeutic combination of the combined or separate embodiments 29-40, wherein the tumor-directed antibody is selected from an anti-CD19 antibody, an anti-CD20 antibody, and an anti-CD38 antibody.
Embodiment 42. The therapeutic combination of the combined or separate embodiments 29-40, wherein the tumor-directed antibody that binds specifically to a glycoprotein is selected from an anti-SLAMF7 antibody, an anti-EpCAM antibody, an anti-gpA3 antibody 3, and an anti-FBP antibody.
Embodiment 43. The therapeutic combination of the combined or separate embodiments 29-40, wherein the tumor-directed antibody that binds specifically to a growth factor is selected from an anti-VEGF antibody, an anti-VEGFR antibody, and an anti-EGFR antibody.
Embodiment 44. The therapeutic combination of the combined or separate embodiments 29-40, wherein the tumor-directed antibody is an IgG antibody.
Embodiment 45. The therapeutic combination of the combined or separate embodiments 29-40, wherein the tumor-directed antibody is selected from the group consisting of daratumumab, rituximab, cetuximab and trastuzumab.
Embodiment 46. The therapeutic combination of the combined or separate embodiments 29-45, wherein the cancer is a solid cancer.
Embodiment 47. The therapeutic combination of the combined or separate embodiments 29-46, wherein the solid cancer is selected from the group consisting of breast cancer, ovarian cancer, colon cancer, colorectal cancer, gastric cancer, malignant melanoma, liver cancer, small cell lung cancer, non-small cell lung cancer, thyroid cancers, kidney cancer, cancer of the bile duct, brain cancer, cervical cancer, maxillary sinus cancer, bladder cancer, esophageal cancer, Hodgkin's disease and adrenocortical cancer, including metastatic forms of any of the foregoing.
Embodiment 48. The therapeutic combination of the combined or separate embodiments 29-45, wherein the cancer is a hematological malignancy.
Embodiment 49. The therapeutic combination of the combined or separate embodiments 29- and 48, wherein the hematological malignancy is selected from the group consisting of multiple myeloma, non-Hodgkin lymphoma, leukemia and lymphoma.
Embodiment 50. The therapeutic combination of the combined or separate embodiments 29- and 48-49, wherein the cancer is a B cell malignancy.
Embodiment 51. A kit comprising the therapeutic combination of the combined or separate embodiments 29-50, accompanied by instructions for use.
Embodiment 52. The kit of embodiment 51, wherein the long-acting IL-15 receptor agonist and the monoclonal antibody are each contained in one or more individual unit dosage forms.
Embodiment 53. The kit of embodiment 51, wherein the long-acting IL-15 receptor agonist and the monoclonal antibody are each contained in the same unit dosage form.

SEQUENCE LISTING

```
SEQ ID NO: 1 (rhIL-15)
                10 20 30 40 50 60
NWVNVISDLK KIEDLIQSMH IDATLYTESD VHPSCKVTAM KCFLLELQVI SLESGDASIH
                70 80 90 100 110
DTVENLIILA NNSLSSNGNV TESGCKECEE LEEKNIKEFL QSFVHIVQMF INTS
SEQ ID NO: 2
-1 10 20 30 40 50 60
  M NWVNVISDLK KIEDLIQSMH IDATLYTESD VHPSCKVTAM KCFLLELQVI SLESGDASIH
                      70 80 90 100 110
      DTVENLIILA NNSLSSNGNV TESGCKECEE LEEKNIKEFL QSFVHIVQMF INTS
SEQ ID NO: 3
                10 20 30 40 50 60
MRISKPHLRS ISIQCYLCLL LNSHFLTEAG IHVFILGCFS AGLPKTEANW VNVISDLKKI
                70 80 90 100 110 120
EDLIQSMHID ATLYTESDVH PSCKVTAMKC FLLELQVISL ESGDASIHDT VENLIILANN
              130 140 150 160
SLSSNGNVTE SGCKECEELE EKNIKEFLQS FVHIVQMFIN TS
```

It is claimed:

1. A method of treating a subject having cancer, the method comprising:
administering to the subject
(a) a long-acting IL-15 receptor agonist having a structure: 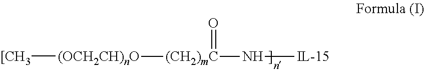
wherein IL-15 is an interleukin-15 moiety, n is an integer from about 150 to about 3,000, m is an integer selected from 2, 3, 4, and 5, n′ is 1, and ˜NH˜ represents an amino group of the IL-15 moiety; and
(b) a monoclonal antibody that binds specifically to a tumor antigen selected from a phosphoprotein, a transmembrane protein, a glycoprotein, a glycolipid, and a growth factor, wherein the monoclonal antibody has antibody dependent cellular cytotoxicity (ADCC) as a mechanism of action;
wherein steps (a) and (b) are carried out concurrently or sequentially and in any order.

2. The method of claim 1, wherein the long-acting IL-15 receptor agonist is a pharmaceutically acceptable salt.

3. The method of claim 1 or 2, wherein (m) in Formula (I) is 2 or 3.

4. The method of any one of claims 1-3, wherein (m) in Formula (I) is 3.

5. The method of any one of claims 1-4, wherein (n) in Formula (I) has a value of about 909.

6. The method of any one of claims 1-5, wherein the cancer is a solid cancer.

7. The method of claim 6, wherein the solid cancer is selected from the group consisting of breast cancer, ovarian cancer, colon cancer, colorectal cancer, gastric cancer, malignant melanoma, liver cancer, small cell lung cancer, non-small cell lung cancer, thyroid cancers, kidney cancer, cancer of the bile duct, brain cancer, cervical cancer, maxillary sinus cancer, bladder cancer, esophageal cancer, Hodgkin's disease and adrenocortical cancer, including metastatic forms of any of the foregoing.

8. The method of any one of claims 1-5, wherein the cancer is a hematological malignancy.

9. The method of claim 8, wherein the hematological malignancy is selected from the group consisting of multiple myeloma, non-Hodgkin lymphoma, leukemia and lymphoma.

10. The method of any one of claims 1-9, wherein step (a) is carried out prior to step (b).

11. The method of any one of claims 1-9, wherein step (b) is carried out prior to step (a).

12. The method of any one of claims 1-9, wherein step (a) and step (b) are carried out concurrently or substantially concurrently.

13. The method of any one of claims 1-12, wherein said administering is effective to stimulate NK activation to an extent greater than observed when the long-acting IL-15 receptor agonist is administered as a single agent, as measured in a suitable animal model.

14. The method of any one of claims 1-13, wherein said administering is effective to stimulate NK proliferation to an extent greater than observed when the long-acting IL-15 receptor agonist is administered as a single agent, as measured in a suitable animal model.

15. The method of any one of claims 1-14, wherein said administering is effective to support CD8+ T-cell survival and memory formation to an extent greater than observed when the long-acting IL-15 receptor agonist is administered as a single agent, as measured in a suitable animal model.

16. The method of any one of claims 1-15, wherein the long-acting IL-15 receptor agonist is administered subcutaneously.

17. The method of any one of claims 1-16, wherein the monoclonal antibody is administered intravenously.

18. The method of any one of claims 1-17, wherein the monoclonal antibody is selected from an anti-CD19 antibody, an anti-CD20 antibody, and an anti-CD38 antibody.

19. The method of any one of claims 1-17, wherein the monoclonal antibody that binds specifically to a glycoprotein is selected from an anti-SLAMF7 antibody, an anti-EpCAM antibody, an anti-gpA3 antibody 3, and an anti-FBP antibody.

20. The method of any one of claims 1-17, wherein the monoclonal antibody that binds specifically to a growth factor is selected from an anti-VEGF antibody, an anti-VEGFR antibody, and an anti-EGFR antibody.

21. The method of any one of claims 1-17, wherein the monoclonal antibody is an IgG antibody.

22. The method of any one of claims 1-17, wherein the monoclonal antibody is selected from the group consisting of daratumumab, rituximab, cetuximab and trastuzumab.

23. A therapeutic combination for use in treating cancer, comprising
(a) a long-acting IL-15 receptor agonist having a structure: 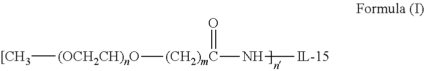
wherein IL-15 is an interleukin-15 moiety, n is an integer from about 150 to about 3,000, m is an integer selected from 2, 3, 4, and 5, n′ is 1, and ˜NH˜ represents an amino group of the IL-15 moiety; and
(b) a monoclonal antibody that binds specifically to a tumor antigen selected from a phosphoprotein, a transmembrane protein, a glycoprotein, a glycolipid, and a growth factor, wherein the monoclonal antibody includes antibody dependent cellular cytotoxicity (ADCC) as a mechanism of action.

24. The therapeutic combination of claim 23, wherein the long-acting receptor agonist is a pharmaceutically acceptable salt.

25. The therapeutic combination of claim 23 or 24, wherein the long-acting IL-15 receptor agonist has a structure as described in any one of claims 3, 4, or 5.

26. A kit comprising the therapeutic combination of any one of claims 23-25, accompanied by instructions for use, wherein the long-acting IL-15 receptor agonist and the monoclonal antibody are each contained in one or more individual unit dosage forms.

27. Use of a long-acting receptor agonist to enhance NK-cell mediated antibody-dependent cellular toxicity when administered with an anti-tumor antibody.


SEQUENCE LISTING (3 sequences)

SEQ ID NO: 1 - protein, 114 aa, Artificial Sequence
  Synthetic protein
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2 - protein, 115 aa, Artificial Sequence
  Synthetic protein
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 3 - protein, 162 aa, Artificial Seqeunce
  Synthetic protein
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS